(19) United States
(12) Patent Application Publication
(10) Pub. No.: US 2023/0382947 A1
(43) Pub. Date: Nov. 30, 2023

(54) METHODS FOR PROXIMITY MEDIATED COUPLING OF A FIRST AGENT TO A SECOND AGENT

(71) Applicant: UNIVERSITEIT GENT, Gent (BE)

(72) Inventors: Alex MANICARDI, Novellara (IT); Annemieke MADDER, Massemen (BE)

(73) Assignee: UNIVERSITEIT GENT, Gent (BE)

(21) Appl. No.: 18/026,441

(22) PCT Filed: Oct. 13, 2021
(86) PCT No.: PCT/EP2021/078367

§ 371 Date: Mar. 15, 2023

(30) Foreign Application Priority Data
Oct. 13, 2020 (EP) 20201595.4

(51) Int. Cl. C07K 1/107; C07H 21/04

(52) U.S. Cl. CPC C07K 1/1075; C07H 21/04

(57) ABSTRACT

The present invention relates to a method for covalently binding a first agent and a second agent, the first agent comprising a first recognition element, wherein the first recognition element comprises an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl; the second agent comprising a second recognition element, wherein the second recognition element comprises a nucleophilic moiety selected from a hydrazine moiety, an aminooxy moiety, an aminosulfanyl moiety or a hydroxylamine moiety; wherein: (A) the first recognition element and the second recognition element are capable of non-covalently binding to each other such that the 1,4-dioxo moiety and the nucleophilic moiety are brought in proximity; the method comprising contacting the first agent with the second agent, thereby covalently binding the 1,4-dioxo moiety and the nucleophilic moiety; or (B) the first recognition element and the second recognition element are capable of non-covalently binding to a third recognition element such that the 1,4-dioxo moiety and the nucleophilic moiety are brought in proximity; the method comprising contacting the first agent with the second agent and the third recognition element, thereby covalently binding the 1,4-dioxo moiety and the nucleophilic moiety; wherein the first recognition element is a peptide nucleic acid (PNA), a peptide, a peptidomimetic, an oligonucleotide, an oligonucleotide mimic, or a combination thereof; the second recognition element is a PNA, a peptide, a peptidomimetic, an oligonucleotide, an oligonucleotide mimic, or a combination thereof; and the third recognition element is a nucleic acid, an oligonucleotide, an oligonucleotide mimic, a PNA, a protein, a peptide, a cyclodextrin, a cucurbituril, a cyclophane, or a combination thereof. The invention further provides related products including kits of parts.

### FIELD OF THE INVENTION

The present invention is broadly in the field of bio-orthogonal chemistry, more precisely in the field of orthogonal methods for coupling or ligation of a first agent to a second agent. In particular, the invention concerns a method coupling of a first agent, such as a first biomolecule, to a second agent, such as a second biomolecule or a surface, and this without the need for external stimuli or without the need for highly reactive and possible unstable groups or reagents. Hence, the invention concerns a mild ligation technique.

### BACKGROUND OF THE INVENTION

In biochemistry, standard coupling strategies are normally based either on reactions that involve highly reactive compounds or on pro-reactive systems. Highly reactive compounds typically show a reduced lifetime due to product degradation. Commonly used examples of such coupling reactions involving reactive compounds are:
Copper free 1,3-dipolar cycloaddition (copper free AAC);
Inverse electron-demand Diels-Alder;
Staudinger ligation: also including traceless Staudinger ligation;
Native chemical ligation;
Michael addition to α,β-unsaturated systems (e.g. thiols and maleimides);
Pictet-Spengler, including aza-Pictet-Spengler.

Most of these methodologies are affected by the hydrolysis or oxidation of one of the reagents leading to by-products that are no longer active. In particular, the strained alkynes employed in the copper free AAC are difficult to synthesize and they hydrolyse fast in aqueous media or they can easily be oxidized by air. The tetrazines employed in the inverse electron-demand Diels-Alder reaction tend to be sensitive to nucleophilic attack and release of nitrogen. Phosphines, thiols and aldehydes can oxidize overtime. Thioesters and seleno-compounds are also sensitive to hydrolysis or prone to nucleophilic attacks.

The pro-reactive systems on the other hand, are activated via specific triggers, which may be chemical or physical. These triggers can lead to damage to sensitive systems of interest. A typical example of such a coupling strategy is the copper-catalysed 1,3-dipolar cycloaddition (CuAAC). Indeed, CuAAC requires a source of copper (I) that is toxic and can induce hydrolysis of some biological components (e.g. DNA and RNA hydrolysis). Further, traces of Cu can be found in the final conjugates which can interfere with biological assays and/or the systems of interest.

Finally, some strategies just rely on the recognition ability of two counterparts without forming a stable strong bond, and thus dissociation can happen over time or when conditions change. Examples of these approaches comprise oligonucleotide tags, His-tag, biotin-streptavidin interaction, etc.

WO 93/18052 concerns covalent cross-linkages for two oligonucleotide strands or for first and second regions of a single oligonucleotide strand to connect sugar moieties of nucleotides on the respective strands or the regions of the single strand. The cross-linkages are connected to at least one strand or region via a space-spanning group. The cross-linked nucleic acid of WO 93/18052 comprises a covalent cross-linkage between first and second active functional groups, wherein the first active functional group is an aldehyde, a protected aldehyde or an aldehyde precursor, and wherein the second active functional group is an amine, hydrazine, hydroxylamine, semicarbazide, thiosemicarbazide, hydrazide, alcohol or thiol.

Arian et al. (JACS, 2014, 136, 3176-3183) demonstrate that small molecules containing a 1,4-dicarbonyl irreversibly inhibit the lyase activity of DNA polymerase p. As shown in Scheme 3 on p. 3177, the 1,4-dicarbonyl is an 1,4-dialdehyde namely 1,4-dioxobutane (DOB).

Zatsepin et al. (Tetrahedron Letters, 2006, 47, 5515-5518) concerns the synthesis of 2′-hydrazine oligonucleotides and their conjugation with aldehydes and 1,3-diketones.

Hamoud et al. (Org. Biomol. Chem., 2018, 16, 1760-1769) describes an N-Heterocyclic Carbene (NHC) catalyzed biomimetic Stetter reaction as a bioconjugation reaction to sensitive nucleoside-type biomolecules to provide pyrrole linked nucleolipids. The 1,4-diketone as shown in Scheme 1 is reacted with propylamine under Paal-Knorr conditions requiring high temperatures (70° C.), long reaction times (5-8 h), and acid catalysis (p-Toluenesulfonic acid or PTSA).

There is therefore a need for further an/or improved coupling methods for a first agent, such as a biomolecule, to a second agent, such as a biomolecule, a fluorescent moiety, a label, a small molecule, or a surface. Preferably, these coupling methods are chemoselective. Preferably, these coupling methods are highly site-specific. Preferably, these coupling methods do not need to be chemically triggered. Preferably, these coupling methods are catalyst free. Preferably, these coupling methods do not need to be physically triggered. Preferably, these coupling methods work without external stimuli. Preferably, these coupling methods work under physiological conditions. Preferably, these coupling methods work at low concentrations. Preferably, these coupling methods work without denaturing biomolecules. Preferably, these coupling methods work with very robust functional groups that are stable in a broad range of environments, such as low or high concentration, different pH, different temperatures, in solution, on surface, etc. Preferably, these coupling methods work with functional groups that do not degrade under physiological conditions. Preferably, these coupling methods allow ligation to a surface. Preferably, these coupling methods provide an irreversible coupling.

### SUMMARY OF THE INVENTION

The present inventors have realized a method for covalently binding a first agent and a second agent based on a chemo-selective reaction triggered by the non-covalent recognition of two or more elements bringing a 1,4-dioxo moiety and nucleophilic moiety into proximity.

Accordingly, the present inventors have found a method for covalently binding a first agent and a second agent, the first agent comprising a first recognition element, wherein the first recognition element comprises an 1,4-dioxo moiety having a structure of Formula IA, IB or IC, wherein R¹² is C₁₋₃alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl;

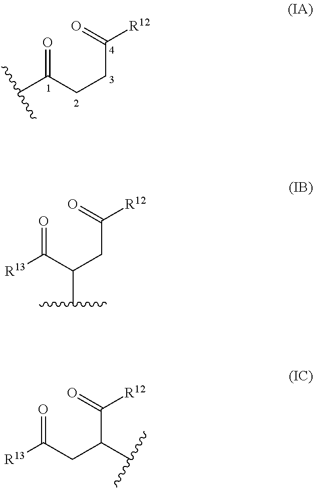

the second agent comprising a second recognition element, wherein the second recognition element comprises a nucleophilic moiety selected from a hydrazine moiety, an aminooxy moiety, an aminosulfanyl moiety or a hydroxylamine moiety;
wherein:
(A) the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the first recognition element and the second recognition element are capable of non-covalently interacting with each other such that the 1,4-dioxo moiety and the nucleophilic moiety are brought in proximity; the method comprising contacting the first agent with the second agent, thereby covalently binding the 1,4-dioxo moiety and the nucleophilic moiety; or
(B) the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, the third recognition element is a nucleic acid, an oligonucleotide, an oligonucleotide mimic, a PNA, a protein, a peptide, a cyclodextrin, a cucurbituril, a cyclophane, or a combination thereof, and the first recognition element and the second recognition element are capable of non-covalently interacting with a third recognition element such that the 1,4-dioxo moiety and the nucleophilic moiety are brought in proximity; the method comprising contacting the first agent with the second agent and the third recognition element, thereby covalently binding the 1,4-dioxo moiety and the nucleophilic moiety.

Such methods for covalently binding a first agent and a second agent allow proximity induced covalent bond formation between the 1,4-dioxo moiety and the nucleophilic moiety. The non-covalent interaction of the recognition elements, which brings the 1,4-dioxo moiety and the nucleophilic moiety in proximity of each other may increase the reactivity and/or reaction rate between the 1,4-dioxo moiety and the nucleophilic moiety. This proximity induced binding may allow to establish a covalent bond between the first agent and the second agent without the need of a trigger, such as a catalyst and/or activation agent. Such methods for covalently binding a first agent and a second agent allow to form an irreversible covalent linkage.

The recognition elements in the methods, may allow a highly specific coupling strategy, by only bringing the 1,4-dioxo moiety and the nucleophilic moiety in close proximity when an adduct is formed between the relevant recognition elements.

The present method further uses functional groups (the 1,4-dioxo moiety as taught herein and the nucleophilic moiety as taught herein) that are robust and stable in a broad range of conditions and additionally do not require activation in order to form the ligation product due to the proximity which is ensured by the binding of the recognition elements.

The 1,4-dioxo moiety as described herein and the nucleophilic moiety as described herein are highly stable especially under physiological conditions. Further, the 1,4-dioxo moiety as described herein and the nucleophilic moiety as described herein do not interfere with the bioactivity of the first agent and the second agent.

Furthermore, the present methods advantageously allow the coupling of a first agent to a second agent under physiological conditions such as physiological pH, temperature, and pressure, and hence the present method can be applied intracellularly. In addition, coupling of a first agent to a second agent according to the methods of the present invention is efficient and results in high yields of conjugates. The kinetics of the present method are satisfying even at low reagent concentrations. Further, the present methods result in the formation of stable conjugates.

The ensuing statements provide additional illustration of certain aspects and embodiments that have been disclosed in accordance with the present invention:
1. A method for covalently binding a first agent and a second agent, the first agent comprising a first recognition element (covalently bound to the first agent), wherein the first recognition element comprises an 1,4-dioxo moiety having a structure of Formula IA, IB or IC, wherein R¹² is C₁₋₃₀alkyl or C₂₋₃₀alkenyl; and R¹³, if present, is hydrogen, C₁₋₃₀alkyl or C₂₋₃₀alkenyl;

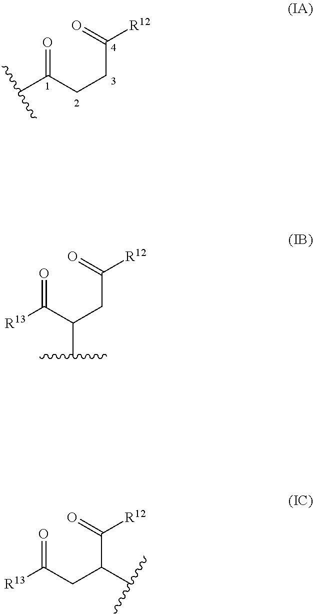

the second agent comprising a second recognition element (covalently bound to the second agent), wherein the second recognition element comprises a nucleophilic moiety selected from a hydrazine moiety, an aminooxy moiety, an aminosulfanyl moiety or a hydroxylamine moiety; wherein:
(A) the first recognition element and the second recognition element are capable of non-covalently binding to each other such that the 1,4-dioxo moiety and the nucleophilic moiety are brought in proximity; the method comprising contacting the first agent with the second agent, thereby covalently binding the 1,4-dioxo moiety and the nucleophilic moiety; or
(B) the first recognition element and the second recognition element are capable of non-covalently binding to a third recognition element such that the 1,4-dioxo moiety and the nucleophilic moiety are brought in proximity; the method comprising contacting the first agent with the second agent and the third recognition element, thereby covalently binding the 1,4-dioxo moiety and the nucleophilic moiety.
2. The method according to statement 1, comprising the prior steps of:
   providing an agent comprising a first recognition element (covalently bound to the first agent), wherein the first recognition element comprises a furyl moiety having a structure of Formula IIA, IIB, or IIC, wherein R¹² is C₁₋₃₀alkyl or C₂₋₃₀alkenyl; and R¹³, if present, is hydrogen, C₁₋₃₀alkyl or C₂₋₃₀alkenyl;

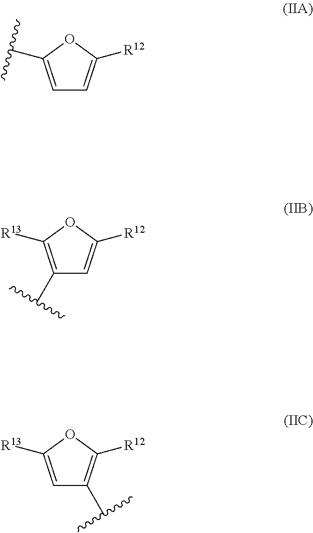

hydrolysing the furyl moiety, thereby obtaining the first agent comprising a 1,4-dioxo moiety.
3. A method for covalently binding a first agent and a second agent, the first agent comprising a first recognition element (covalently bound to the first agent), wherein the first recognition element comprises an 1,4-dioxo moiety having a structure of Formula I, wherein R¹² is C₁₋₃₀alkyl or C₂₋₃₀alkenyl;

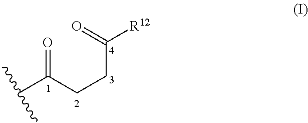

the second agent comprising a second recognition element (covalently bound to the second agent), wherein the second recognition element comprises a nucleophilic moiety selected from a hydrazine moiety, an aminooxy moiety, an aminosulfanyl moiety or a hydroxylamine moiety;
wherein the first recognition element and the second recognition element are capable of non-covalently binding to each other such that the 1,4-dioxo moiety and the nucleophilic moiety are brought in proximity;
the method comprising contacting the first agent with the second agent, thereby covalently binding the 1,4-dioxo moiety and the nucleophilic moiety.
4. The method according to any one of statements 1 to 3, comprising the steps of:
   providing the first agent and the second agent; and
   contacting the first agent with the second agent, thereby covalently binding the 1,4-dioxo moiety and the nucleophilic moiety.
5. The method according to any one of statements 1 to 4, wherein the first recognition element and the second recognition element are capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å.
6. A method for covalently binding a first agent and a second agent, the first agent comprising a first recognition element (covalently bound to the first agent), wherein the first recognition element comprises a 1,4-dioxo moiety having a structure of Formula I, wherein R¹² is C₁₋₃₀alkyl or C₂₋₃₀alkenyl;
   the second agent comprising a second recognition element (covalently bound to the second agent), wherein the second recognition element comprises a nucleophilic moiety selected from a hydrazine moiety, an aminooxy moiety, an aminosulfanyl moiety or a hydroxylamine moiety;
   wherein the first recognition element and the second recognition element are capable of non-covalently binding to a third recognition element such that the 1,4-dioxo moiety and the nucleophilic moiety are brought in proximity;
   the method comprising contacting the first agent with the second agent and the third recognition element, thereby covalently binding the 1,4-dioxo moiety and the nucleophilic moiety.
7. The method according to any one of statements 1 to 6, comprising the steps of:
   providing the first agent, the second agent and the third recognition element; and
   contacting the first agent with the second agent and the third recognition element, thereby covalently binding the 1,4-dioxo moiety and the nucleophilic moiety.
8. The method according to any one of statements 1 to 7, comprising the prior steps of:
   providing an agent comprising a first recognition element (covalently bound to the first agent), wherein the first recognition element comprises a furyl moiety having a structure of Formula II, wherein R¹² is C₁₋₃₀alkyl or C₂₋₃₀alkenyl;

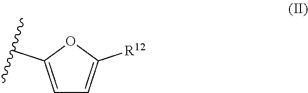

hydrolysing the furyl moiety, thereby obtaining the first agent.
9. The method according to any one of statements 1 to 8, wherein the first recognition element and the second recognition element are capable of non-covalently binding to a third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å.
10. The method according to any one of statements 1 to 9, wherein the first recognition element and/or the second recognition element is a peptide nucleic acid (PNA), a peptide, a peptidomimetic, an oligonucleotide, an oligonucleotide mimic, or a combination thereof.
11. The method according to any one of statements 1 to 10, wherein the third recognition element is a nucleic acid, an oligonucleotide, an oligonucleotide mimic, a PNA, a protein, a peptide, a cyclodextrin, a cucurbituril, a cyclophane, or a combination thereof.
12. The method according to any one of statements 1 to 11, wherein the first recognition element and the second recognition element are complementary peptide nucleic acids; or wherein the first recognition element and the second recognition element are peptide nucleic acids, and the third recognition element is an oligonucleotide, wherein the first recognition element and the second recognition element are complementary to the third recognition element.
13. The method according to any one of statements 1 to 11, wherein the first recognition element and the second recognition element are coiled coil peptides.
14. The method according to any one of statements 1 to 13, wherein the 1,4-dioxo moiety comprises a group having a structure of Formula III or IIIa, wherein:

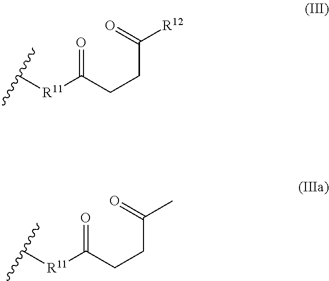

R¹¹ is C₁₋₁₅alkyl, C₃₋₁₅alkenyl, C₆₋₁₅ aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; and
   R¹² is C₁₋₃₀alkyl or C₂₋₃₀alkenyl.
15. The method according to any one of statements 1 to 14, wherein R¹² is C₁₋₃₀alkyl or C₃₋₃₀alkenyl, and R¹³ if present is hydrogen, C₁₋₃₀alkyl or C₃₋₃₀alkenyl; or wherein R¹² is methyl, and R¹³ if present is hydrogen or methyl.
16. The method according to any one of statements 1 to 15, wherein the nucleophilic moiety comprises a group having a structure of Formula VII, VIII, or IX, wherein:

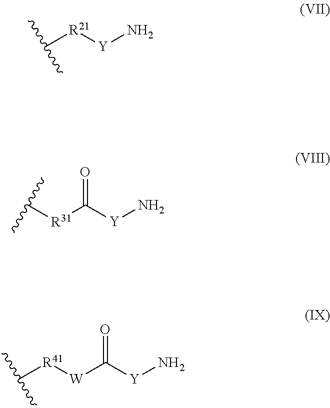

Y is NR¹, O or S, wherein R¹ is hydrogen, C₁₋₃₀alkyl, or C₆₋₂₀aryl;
R²¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
R³¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
Q is O, S, or NR⁴, wherein R⁴ is hydrogen, C₁₋₃₀alkyl or C₆₋₂₀aryl;
R⁴¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
W is NR⁵, O, or S, wherein R⁵ is hydrogen, C₁₋₃₀alkyl or C₆₋₂₀aryl.
17. The method according to any one of statements 1 to 16, wherein the first agent is a protein, a polypeptide, a peptide, a nucleic acid, a polysaccharide, a lipid, a small molecule, a polymer, a labeling reagent, a solid surface, a particle, or a combination thereof.
18. The method according to any one of statements 1 to 17, wherein the second agent is a protein, a polypeptide, a peptide, a nucleic acid, a polysaccharide, a lipid, a small molecule, a polymer, a labeling reagent, a solid surface, a particle, or a combination thereof.
19. The method according to any one of statements 1 to 18, wherein the first agent is contacted with the second agent, and optionally the third recognition element, without the addition of an activation signal.
20. The method according to any one of statements 1 to 19, wherein the first recognition element, the second recognition element, and optionally the third recognition element are contacted in a concentration of at least 2.5 nM, preferably at least 10 nM, at least 100 nM, at least 1 μM, at least 10 μM, or at least 100 μM.
21. The method according to any one of statements 1 to 20, wherein the method is performed:
   in an aqueous solution;
   at physiological conditions;
   at a temperature ranging from 5 to 50° C., preferably at a temperature ranging from 20 to 40° C.;
   at a pH ranging from about 3 to about 11, preferably at a pH ranging from about 4 to about 8;
   in the absence of a catalyst (e.g. acid catalysis); and/or
   in the absence of a dehydrating agent.
22. A kit of parts comprising:
   a) a first recognition element comprising an 1,4-dioxo moiety having a structure of Formula IA, IB or IC, wherein R¹² is C₁₋₃₀alkyl or C₂₋₃₀alkenyl; and R¹³, if present, is hydrogen, C₁₋₃₀alkyl or C₂₋₃₀alkenyl;
   b) a second recognition element comprising a hydrazine moiety, an aminooxy moiety, an aminosulfanyl moiety, or a hydroxylamine moiety;
   wherein the first recognition element and the second recognition element are capable of non-covalently binding to each other such that the 1,4-dioxo moiety and the nucleophilic moiety are brought in proximity.
23. A kit of parts comprising:
   a′) a first recognition element comprising an 1,4-dioxo moiety having a structure of Formula IA, IB or IC, wherein R¹² is C₁₋₃₀alkyl or C₂₋₃₀alkenyl; and R¹³, if present, is hydrogen, C₁₋₃₀alkyl or C₂₋₃₀alkenyl;
   b′) a second recognition element comprising a hydrazine moiety, an aminooxy moiety, an aminosulfanyl moiety, or a hydroxylamine moiety;
   c′) a third recognition element capable of non-covalently binding to the first recognition element and the second recognition element such that the 1,4-dioxo moiety and the nucleophilic moiety are brought in proximity.
24. A kit of parts comprising:
   a) a first recognition element comprising an 1,4-dioxo moiety having a structure of Formula I, wherein R¹² is C₁₋₃₀alkyl or C₂₋₃₀alkenyl; and
   b) a second recognition element comprising a hydrazine moiety, an aminooxy moiety, an aminosulfanyl moiety, or a hydroxylamine moiety;
   wherein the first recognition element and the second recognition element are capable of non-covalently binding to each other such that the 1,4-dioxo moiety and the nucleophilic moiety are brought in proximity.
25. A kit of parts comprising:
   a′) a first recognition element comprising an 1,4-dioxo moiety having a structure of Formula I, wherein R¹² is C₁₋₃₀alkyl or C₂₋₃₀alkenyl;
   b′) a second recognition element comprising a hydrazine moiety, an aminooxy moiety, an aminosulfanyl moiety, or a hydroxylamine moiety;
   c′) a third recognition element capable of non-covalently binding to the first recognition element and the second recognition element such that the 1,4-dioxo moiety and the nucleophilic moiety are brought in proximity.
26. The kit of parts according to any one of statements 22 to 25, wherein the first recognition element and the second recognition element are capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å, or wherein the first recognition element and the second recognition element are capable of non-covalently binding to the third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å.
27. The kit of parts according to any one of statements 22 to 26, wherein the first recognition element and/or the second recognition element is a peptide nucleic acid (PNA), a peptide, a peptidomimetic, an oligonucleotide, an oligonucleotide mimic, or a combination thereof.
28. The kit of parts according to any one of statements 23 to 27, wherein the third recognition element is a nucleic acid, an oligonucleotide, an oligonucleotide mimic, a PNA, a protein, a peptide, a cyclodextrin, a cucurbituril, a cyclophane, or a combination thereof.
29. The kit of parts according to any one of statements 22 to 28, wherein the first recognition element and the second recognition element are complementary peptide nucleic acids, or wherein the first recognition element and the second recognition element are coiled coil peptides.
30. The kit of parts according to any one of statements 23 to 29, wherein the first recognition element and the second recognition element are peptide nucleic acids, and the third recognition element is an oligonucleotide, wherein the first recognition element and the second recognition element are complementary to the third recognition element.
31. The kit of parts according to any one of statements 22 to 30, wherein the first recognition element comprises a 1,4-dioxo moiety having a structure of Formula III or IIIa, wherein:
   R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; and
   R¹² is C₁₋₃₀alkyl or C₂₋₃₀alkenyl.
32. The kit of parts according to any one of statements 22 to 31, wherein R¹² is C₁₋₃₀alkyl or C₃₋₃₀alkenyl, and R¹³ if present is hydrogen, C₁₋₃₀alkyl or C₃₋₃₀alkenyl; or wherein R¹² is methyl, and R¹³ if present is hydrogen or methyl.
33. The kit of parts according to any one of statements 22 to 32, wherein the nucleophilic moiety comprises a group having a structure of Formula VII, VIII, or IX, wherein:
   Y is NR¹, O or S, wherein R¹ is hydrogen, C₁₋₃₀alkyl, or C₆₋₂₀aryl;
   R²¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆ alkoxy;
   R³¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
   Q is O, S, or NR⁴, wherein R⁴ is hydrogen, C₁₋₃₀alkyl or C₆₋₂₀aryl;
   R⁴¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
   W is NR⁵, O, or S, wherein R⁵ is hydrogen, C₁₋₃₀alkyl or C₆₋₂₀aryl.
34. A peptide nucleic acid (PNA) comprising an 1,4-dioxo moiety having a structure of Formula IA, IB or IC, wherein R¹² is C₁₋₃₀alkyl or C₂₋₃₀alkenyl; and R¹³, if present, is hydrogen, C₁₋₃₀alkyl or C₂₋₃₀alkenyl.
35. A peptide comprising an 1,4-dioxo moiety having a structure of Formula IA, IB or IC, wherein R¹² is C₁₋₃₀alkyl or C₂₋₃₀alkenyl; and R¹³, if present, is hydrogen, C₁₋₃₀alkyl or C₂₋₃₀alkenyl.
36. A peptide nucleic acid (PNA) comprising an 1,4-dioxo moiety having a structure of Formula I, wherein R¹² is C₁₋₃₀alkyl or C₂₋₃₀alkenyl.
37. A peptide comprising an 1,4-dioxo moiety having a structure of Formula I, wherein R¹² is C₁₋₃₀alkyl or C₂₋₃₀alkenyl.
38. The PNA according to statement 34 or 36, or the peptide according to statement 35 or 37, comprising a group having a structure of Formula III or IIIa, wherein:
   R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy; and
   R¹² is C₁₋₃₀alkyl or C₂₋₃₀alkenyl.
39. The PNA according to statement 34, 36 or 38, or the peptide according to statement 35, 37 or 38, wherein R¹² is C₁₋₃₀alkyl or C₃₋₃₀alkenyl, and R¹³ if present is hydrogen, C₁₋₃₀ alkyl or C₃₋₃₀alkenyl; or wherein R¹² is methyl, and R¹³ if present is hydrogen or methyl.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### BRIEF DESCRIPTION OF THE FIGURES

FIG. 1 represents a scheme depicting the principle of a method according to an embodiment of the invention for covalently binding a first agent (1) and a second agent (2).

FIG. 2 represents a scheme depicting the principle of a method according to an embodiment of the invention for covalently binding a first agent (1) and a second agent (2) using a third recognition element (30).

FIG. 3 represents a scheme depicting the principle of a method according to an embodiment of the invention for preparing a protein array on a surface.

FIG. 4 represents a schematic overview illustrating the structure of PNAs comprising a 1,4-dioxo moiety: PNA-M1 (SEQ ID NO: 1), PNA-M2 (SEQ ID NO: 2), and PNA-M3 (SEQ ID NO: 3).

FIG. 5 represents a schematic overview illustrating the structure of PNAs comprising a nucleophilic moiety: PNA-A1 (SEQ ID NO: 5), PNA-C1 (SEQ ID NO: 6), PNA-H1 (SEQ ID NO: 7), PNA-Z1 (SEQ ID NO: 8) and PNA-S1 (SEQ ID NO: 9).

FIG. 6 shows the USDS-PAGE analysis of ligation experiments according to an embodiment of the invention with a) amine-modified PNA-A1; b) amide-modified PNA-C1; c) hydrazine-modified PNA-H1; d) hydrazide-modified PNA-Z1; e) semicarbazide-modified PNA-S1. Lanes: (1) color; (2) with fully matched PNA-M1; (3) with fully matched PNA-M1+methylhydrazine quenching; (4) with doubly mismatched PNA-M2; (5) with doubly mismatched PNA-M2+methylhydrazine quenching; (6) with scrambled PNA-M3; (7) with scrambled PNA-M3+methylhydrazine quenching.

FIG. 7 shows ESI-MS characterization of the ligation production formed in Example 1; a) fully matched PNA-M1+hydrazine-modified PNA-H1; b) PNA-M1+semicarbazide-modified PNA-S1; dotted-line encircled multicharged signals correspond to the hydrated form of the product, full-line encircled multicharged signals correspond to the product of N—N bond hydrolysis during the ionization process; X-axis: m/z; Y-axis: relative intensity.

FIG. 8 shows (a) the USDS-PAGE analysis and (b) the HPLC-UV analyses of a templated PNA₂:DNA ligation method according to an embodiment of the invention performed between PNA-M3 (SEQ ID NO: 3) and PNA-H1 (SEQ ID NO: 7); DNA-1 (SEQ ID NO: 19), DNA-2 (SEQ ID NO: 20), and DNA-3 (SEQ ID NO: 21) are used as third recognition element; *: ligation product; (c) shows the ESI-MS spectra of the ligation product obtained in the templated PNA₂:DNA ligation method according to an embodiment of the invention performed between PNA-M3 and PNA-H1; X-axis: m/z; Y-axis: relative intensity.

FIG. 9 illustrates the surface templated ligation with PNA-M4. (A) Schematic workflow of the method according to an embodiment of the invention: (i) hybridization of DNA target to surface immobilized PNA; (ii) hybridization of the nucleophile-containing probe and formation of the PNA₂:DNA complex; (iii) overnight incubation and formation of the PNA-PNA:DNA complex; detection of the final ligation product can be performed through incubation with NAv-HRP and evaluation of the resulting peroxidase activity (black box, in detail below) or by fluorescence emission of the TAMRA reporting group (grey box, in detail below). (B) Structure of the nucleophilic probes used for the ligation on surface and probe sequences used for surface experiments. (C) Structure of the DOP-containing PNA-M4 (SEQ ID NO: 4). (D) TMBₒₓ signal generation after overnight incubation of 1 μM solution of PNA-H2 (sequence of the modification on the Lys side chain: SEQ ID NO: 11), PNA-A2 (sequence of the modification on the Lys side chain: SEQ ID NO: 10) or PNA-C2 (sequence of the modification on the Lys side chain: SEQ ID NO: 12) in presence of fully matched DNA-7 and scrambled DNA-13 in a 96-well plate format. (E) TAMRA emission recorded after overnight incubation of 50 nM PNA-H2 in presence of 50 nM of the different DNA sequences in a microarray format. The insert shows the signal generated after overnight incubation of a 100 nM solution of PNA-H2, PNA-A2 or PNA-C2 in presence of 50 nM of fully matched DNA-7 and scrambled DNA-13.

FIG. 10 represents a method for covalent binding of a first recognition element according to an embodiment of the invention and a second recognition element. (A) a schematic representation of the coiled coil structure and disposition of different modification sites based on the 4EPT crystal structure. (B) HPLC trace of the ligation experiment (indicated with *) between 6-DOP-Coil (dark grey, SEQ ID NO: 13) and 1-Hy-Coil (light grey, SEQ ID NO: 14). (C) HPLC trace of the ligation experiment between 6-DOP-Coil (dark grey) and 6-Hy-Coil (light grey, SEQ ID NO: 15). *: ligation product; ABA: 4-acetamidobenzoic acid. All experiments were conducted at 5 μM coil concentration in PBS buffer pH 7.4, 25° C.

FIG. 11 shows the ESI-MS characterization of the ligation product formed by a method according to an embodiment of the invention, using peptide coil-coil interaction for the recognition between the first recognition element (6-DOP-Coil) and the second recognition element (1-Hy-Coil); X-axis: m/z; Y-axis: relative intensity.

FIG. 12 represents a graph illustrating the main surface modification chemistries and modifications of the recognition element employed for covalent immobilization of a recognition element to a solid surface.

FIG. 13 represents a graph illustrating a method for coupling gold nanoparticles to a recognition element, such as a PNA, via double exchange Diels-Alder reaction.

FIG. 14 represents a graph illustrating a method for coupling an agent such as a protein to a recognition element such as a peptide or PNA by native chemical ligation.

FIG. 15 represents a graph illustrating a method according to an embodiment of the invention for covalently binding a first agent to a second agent for immobilization on a support.

FIG. 16 illustrates a method according to an embodiment of the invention for covalent binding of a first recognition element and a second recognition element. (A-D) HPLC traces of the ligation experiments between 6-Xn-Coils (black circle, SEQ ID NO: 32, 33, 34, or 35 for n=1, 2, 3, or 4, respectively) and 1-Hy-Coil (light grey circle, SEQ ID NO: 14). (E-H) HPLC traces of the ligation experiments between 6-Xn-Coils (black circle) and 6-Hy-Coil (light grey diamond, SEQ ID NO: 15). (A,E) Experiment performed with SEQ ID NO: 32. (B,F) Experiment performed with SEQ ID NO: 33. (C,G) Experiment performed with SEQ ID NO: 34. (D,H) Experiment performed with SEQ ID NO: 35. The remainder of the figure provides a schematic representation of the coiled coil structure and disposition of different modification sites. *: ligation product; ABA: 4-acetamidobenzoic acid. All experiments were conducted at 5 μM coil concentration in PBS buffer pH 7.4, 25° C.

FIG. 17 illustrates a method according to an embodiment of the invention for covalent binding of a first recognition element and a second recognition element. (A) HPLC traces of the ligation experiment between 6-X5-Coils (black circle, SEQ ID NO: 36) and 1-Hy-Coil (light grey circle, SEQ ID NO: 14). (B) HPLC traces of the ligation experiment between 6-X5-Coil (black circle, SEQ ID NO: 36) and 6-Hy-Coil (light grey diamond, SEQ ID NO: 15). The remainder of the figure provides a schematic representation of the coiled coil structure and disposition of different modification sites. *: ligation product; ABA: 4-acetamidobenzoic acid. All experiments were conducted at 5 μM coil concentration in PBS buffer pH 7.4, 25° C.

### DETAILED DESCRIPTION OF THE INVENTION

Before the present method and products of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The present inventors have realized a method for covalently binding a first agent and a second agent based on a chemo-selective reaction triggered by the recognition of two or more elements bringing a 1,4-dioxo moiety and nucleophilic moiety into proximity.

Hence, a first aspect of the invention provides a method for covalently binding a first agent and a second agent, the first agent comprising or consisting of a first recognition element, wherein the first recognition element comprises an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl;
the second agent comprising or consisting of a second recognition element, wherein the second recognition element comprises a nucleophilic moiety selected from a hydrazine moiety, an aminooxy moiety, an aminosulfanyl moiety or a hydroxylamine moiety;
wherein the first recognition element is a peptide nucleic acid (PNA), a peptide, a peptidomimetic, an oligonucleotide, an oligonucleotide mimic, or a combination thereof, and the second recognition element is a PNA, a peptide, a peptidomimetic, an oligonucleotide, an oligonucleotide mimic, or a combination thereof;
wherein the first recognition element and the second recognition element are capable of non-covalently interacting with each other such that the 1,4-dioxo moiety and the nucleophilic moiety are brought in proximity;
the method comprising contacting the first agent with the second agent, thereby covalently binding the 1,4-dioxo moiety and the nucleophilic moiety.

A second aspect provides a method for covalently binding a first agent and a second agent, the first agent comprising or consisting of a first recognition element, wherein the first recognition element comprises a 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl;
the second agent comprising or consisting of a second recognition element, wherein the second recognition element comprises a nucleophilic moiety selected from a hydrazine moiety, an aminooxy moiety, an aminosulfanyl moiety or a hydroxylamine moiety;
wherein the first recognition element is a peptide nucleic acid (PNA), a peptide, a peptidomimetic, an oligonucleotide, an oligonucleotide mimic, or a combination thereof; the second recognition element is a peptide nucleic acid (PNA), a peptide, a peptidomimetic, an oligonucleotide, an oligonucleotide mimic, or a combination thereof; and the third recognition element is a nucleic acid, an oligonucleotide, an oligonucleotide mimic, a PNA, a protein, a peptide, a cyclodextrin, a cucurbituril, a cyclophane, or a combination thereof;
wherein the first recognition element and the second recognition element are capable of non-covalently interacting with a third recognition element such that the 1,4-dioxo moiety and the nucleophilic moiety are brought in proximity;
the method comprising contacting the first agent with the second agent and the third recognition element, thereby covalently binding the 1,4-dioxo moiety and the nucleophilic moiety.

The present invention provides methods for the site-selective coupling of a first agent to a second agent.

The terms “coupling”, “binding”, “ligation” or “conjugating” can be used interchangeably herein and refer to linking a first agent to a second agent with a covalent bond.

The term “first agent” as used herein refers to any agent which is of interest to be coupled to a second agent. In the context of the present invention, the nature of the first agent is not limited as long as the first agent comprises or consists of a first recognition element comprising a 1,4-dioxo moiety as taught herein. The first agent may be covalently bound to a first recognition element comprising a 1,4-dioxo moiety as taught herein, or the first agent may be a first recognition element comprising a 1,4-dioxo moiety as taught herein. The methods for covalently coupling a first agent and a first recognition element are described herein below.

The term “second agent” as used herein refers to any agent which is of interest to be coupled to a first agent. In the context of the present invention, the nature of the second agent is not limited as long as the second agent comprises or consists of a second recognition element comprising a nucleophilic moiety as taught herein. The second agent may be covalently bound to a second recognition element comprising a nucleophilic moiety as taught herein, or the second agent may be a second recognition element comprising a nucleophilic moiety as taught herein. The methods for covalently coupling a second agent and a second recognition element are described herein below.

In certain embodiments of the methods as taught herein, the agent, such as the first agent and/or the second agent, may be a protein, a polypeptide, a peptide, a nucleic acid, a polysaccharide, a lipid, a small molecule, a polymer, a labeling reagent, a solid surface, a particle, or a combination thereof.

In certain embodiments of the methods as taught herein, when the agent is or consists of a recognition element, the agent may be a peptide nucleic acid (PNA), a peptide, a peptidomimetic, an oligonucleotide, an oligonucleotide mimic, or a combination thereof. In certain embodiments, when the first agent is or consists of a first recognition element, the first agent may be a peptide nucleic acid (PNA), a peptide, a peptidomimetic, an oligonucleotide, an oligonucleotide mimic, or a combination thereof. In certain embodiments, when the second agent is or consists of a second recognition element, the second agent may be a peptide nucleic acid (PNA), a peptide, a peptidomimetic, an oligonucleotide, an oligonucleotide mimic, or a combination thereof.

In certain embodiments of the methods as taught herein, the agent, such as the first agent and/or the second agent, may be a protein, a polypeptide, a peptide, a peptide nucleic acid (PNA), a peptidomimetic, a nucleic acid, an oligonucleotide, an oligonucleotide mimic, a polysaccharide, a lipid, a small molecule, a polymer, a labeling reagent, a solid surface, a particle, or a combination thereof.

The term “protein” as used herein generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally occurring protein parts that ensue from processing of such full-length proteins.

The term “polypeptide” as used herein generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally occurring polypeptide parts that ensue from processing of such full-length polypeptides.

The term “peptide” as used herein refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

A “peptide bond”, “peptide link” or “amide bond” is a covalent bond formed between two amino acids when the carboxyl group of one amino acid reacts with the amino group of the other amino acid, thereby releasing a molecule of water.

The term “nucleic acid” as used herein typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Exemplary modified nucleobases include without limitation 5-substituted pyrimidines, 6-azapyrimidines and N-2, N-6 and 0-6 substituted purines, including 2-aminopropyladenine, 5-propynyluracil and 5-propynylcytosine. In particular, 5-methylcytosine substitutions have been shown to increase nucleic acid duplex stability and may be preferred base substitutions in for example antisense agents, even more particularly when combined with 2′-O-methoxyethyl sugar modifications. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleic acid molecules comprising at least one ribonucleoside unit may be typically referred to as ribonucleic acids or RNA. Such ribonucleoside unit(s) comprise a 2′-OH moiety, wherein —H may be substituted as known in the art for ribonucleosides (e.g., by a methyl, ethyl, alkyl, or alkyloxyalkyl). Preferably, ribonucleic acids or RNA may be composed primarily of ribonucleoside units, for example, ≥80%, ≥85%, ≥90%, ≥95%, ≥96%, ≥97%, ≥98%, ≥99% or even 100% (by number) of nucleoside units constituting the nucleic acid molecule may be ribonucleoside units. Nucleic acid molecules comprising at least one deoxyribonucleoside unit may be typically referred to as deoxyribonucleic acids or DNA. Such deoxyribonucleoside unit(s) comprise 2′-H. Preferably, deoxyribonucleic acids or DNA may be composed primarily of deoxyribonucleoside units, for example, ≥80%, ≥85%, ≥90%, ≥95%, ≥96%, ≥97%, ≥98%, ≥99% or even 100% (by number) of nucleoside units constituting the nucleic acid molecule may be deoxyribonucleoside units. Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof.

The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The term “polysaccharide” generally refers to a polymer or macromolecule consisting of monosaccharide units joined together by glycosidic bonds.

The term “lipid” generally refers to a polymer or macromolecule that is soluble in nonpolar solvents.

The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The term “polymer” generally refers to a macromolecule composed of many covalently bonded repeated subunits.

In certain embodiments, the agent may be a synthetic polymer; preferably the synthetic polymer being selected from the list comprising polyester (PES), polyethylene terephthalate (PET), polyethylene (PE), high-density polyethylene (HDPE), polyvinyl chloride (PVC), polyvinylidene chloride (PVDC), low-density polyethylene (LDPE), polypropylene (PP), polystyrene (PS) high impact polystyrene (HIPS), polyamides (PA) (Nylons), acrylonitrile butadiene styrene (ABS), polyethylene/acrylonitrile butadiene styrene (PE/ABS), polycarbonate (PC), polycarbonate/acrylonitrile butadiene styrene (PC/ABS), and polyurethanes (PU).

The terms “labelling agent” or “label” refer to any atom, molecule, moiety or biomolecule that may be used to provide a detectable and preferably quantifiable read-out or property.

The terms “solid support” or “solid surface” may be used interchangeably herein.

In certain embodiments, the solid support may be substantially made of a solid support material. In certain embodiments, the solid support material may be any suitable material onto which a recognition element (e.g., PNA or peptide) can be immobilized. In certain embodiments, the solid support may be substantially made of a chemically activated material onto which a recognition element (e.g., PNA or peptide) can be immobilized. In certain embodiments, the solid support material may be any suitable rigid material onto which a recognition element (e.g., PNA or peptide) can be immobilized.

In certain embodiments, the solid support material may a polymeric material or glass. In certain embodiments, the solid support material may be substantially plastic, such as for example polystyrene, polypropylene, polycarbonate, polyethylene glycol (PEG), poly(2-oxazoline), or copolymers thereof.

In certain embodiments, the agent may be a particle such as a microparticle or a nanoparticle.

In certain embodiments, the recognition element may be covalently bound to the agent. In certain embodiments, the first recognition element may be covalently bound to the first agent. In certain embodiments, the second recognition element may be covalently bound to the second agent.

The agent and the recognition element may be covalently associated or bound to each other via (by means of) a direct covalent bond or via (by means of) a linker.

In certain embodiments, the linker may comprise or consist essentially of a polyether, ether, amine, polyamine, or a combination of two or more thereof.

In certain embodiments, the linker may comprise or consist essentially of a poly(_{c1-6}alkyleneoxide), C₁₋₆alkyleneoxide, amine, or poly(iminoC₁₋₆alkylene).

The term “polyether” generally refers to a class of organic compounds that contain more than one ether group (i.e., an oxygen atom connected to two alkyl or aryl groups). Non-limiting examples of polyether to be used as a linker are polyethylene oxide (PEO) (i.e., linked polyethylene glycol (PEG) units), polypropylene oxide (PPO), or a block co-polymer of PEO and PPO.

The term “polyamine” generally refers to a class of organic compounds that contain more than one amine group (i.e., a nitrogen atom connected to two or three alkyl or aryl groups). Non-limiting examples of polyamine to be used as a linker are polyethylene imine (PEI), polypropylene imine (PPI), or a block co-polymer of PEI and PPI.

The recognition element (e.g. the first recognition element as taught herein, or the second recognition element as taught herein) may be introduced into the agent (e.g. in the first agent, or the second agent, respectively) by a chemical reaction, by recombinant molecular genetic techniques, by a cell or translation system, or may be part of the agent (e.g., may be naturally present in the agent or may be part of a commercially available agent).

Methods for introducing a recognition element as taught herein into an agent as taught herein depend on the nature of the recognition element and the nature of the agent, and are as known in the art. The recognition element may be introduced into the agent by a ligation strategy selected form the group consisting of copper free 1,3-dipolar cycloaddition (copper free AAC); inverted electron demanding Diels-Alder; Staudinger ligation such as traceless Staudinger ligation; native chemical ligation; Michael addition to α,β-unsaturated systems (e.g. thiols and maleimides); Pictet-Spengler (including aza-Pictet-Spengler); Copper-catalysed 1,3-dipolar cycloaddition (CuAAC); and furan-oxidation based strategies.

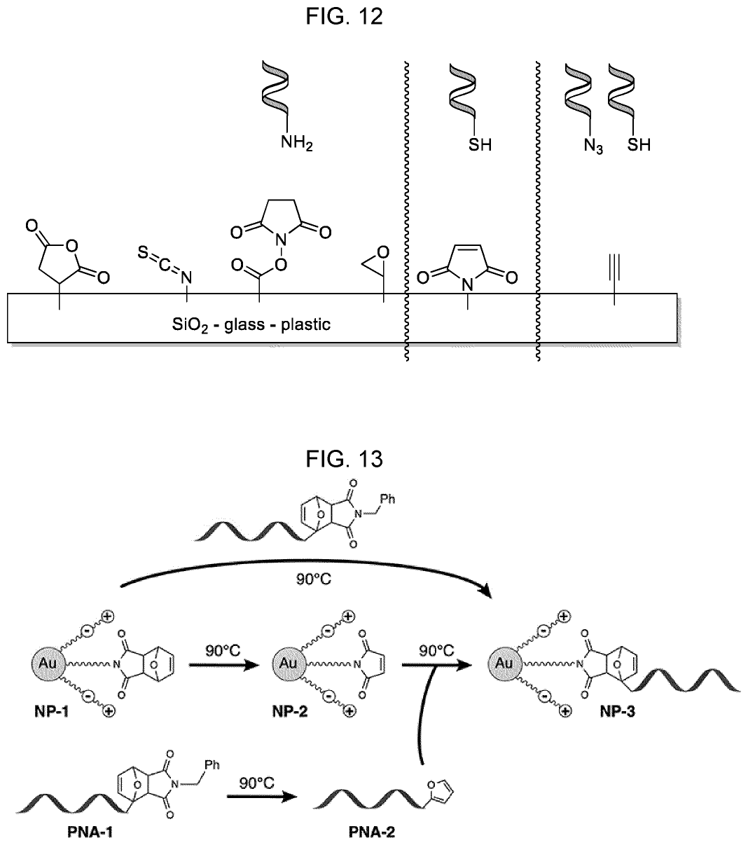

For example, FIG. 12 illustrates the main surface modification chemistries and modifications of the recognition element employed for covalent immobilization of a recognition element to a solid surface. For example, the surface may be modified with succinic anhydride, isothiocyanate, N-hydroxysuccinimide (NHS) ester, or epoxide, and the recognition element may comprise an amine (—NH₂) for covalently coupling to the solid surface. For example, the surface may be modified with maleimide, and the recognition element may comprise a thiol (—SH) for covalently coupling to the solid surface. For example, the surface may be modified with an alkyne, and the recognition element may comprise a thiol or azide (—N₃) for covalently coupling to the solid surface.

For example, the agent may be a solid surface such as a plastic surface comprising succinic anhydride and the recognition element may be a recognition element such as a peptide or a PNA, optionally comprising a spacer such as a (2-(2-aminoeth-oxy)ethoxy)acetyl spacer. For example, the agent may be a solid surface such as a glass surface, comprising N-hydroxysuccinimide and the recognition element may be a recognition element such as a peptide or a PNA, optionally comprising a spacer such as a (2-(2-aminoeth-oxy)ethoxy)acetyl spacer.

Further examples of coupling of PNA on glass surface are described in Calabretta et al., 2009, Mol. BioSyst., 5, 1323-1330; Bertucci et al., 2015, Biosens Bioelectron, 15, 63:248-254.

Further examples of coupling PNA on quartz are provided in Movilli et al., 2018, Bioconjugate Chem., 29 (12), 4110-4118.

Further examples of coupling PNA on silicon/silicon-oxide are provided in Cattani-Scholz et al., 2008, ACS Nano, 2 (8), 1653-1660; Veerbeek et al., 2018, Langmuir, 34 (38), 11395-11404; Movilli et al., 2019, ACS Appl. Polym. Mater., 1 (11), 3165-173.

For example, the agent may be a particle such as a nanoparticle, and the recognition element may be a peptide or a PNA. For example, gold nanoparticles may be coupled to a recognition element, such as a PNA, via double exchange Diels-Alder reaction as shown in FIG. 13. For example, iron (Fe) nanoparticles may be coupled to a recognition element via thiol-maleimide or amino-NHS chemistries.

Further examples of coupling PNA on nanoparticles are described in Galli et al. 2017, RSC Adv., 7, 15500-15512; Cadoni et al., 2020, Frontiers in Chemistry, 8, 4.

For example, the agent may be a protein, a polypeptide or a peptide, and the recognition element may be a peptide or a PNA. The recognition element may be introduced into the agent by use a Sortase A (Shinya et al., 2009, ChemBioChem, 10 (5), 787-798). The recognition motif (LPXTG) may be added to the C-terminus of an agent such as a protein, a polypeptide or a peptide of interest, and an oligo-glycine motif may be added to the N-terminus of the recognition element such as a peptide or PNA to be ligated. The agent and the recognition element may be coupled by the transpeptidase activity of the sortase. For instance, conjugation of PNA to antibodies/proteins via Sortase tag are described in Westerlund et al., 2019, Bioconjugate Chem., 26 (8), 1724-1736; Westerlund et al., 2019, Biomaterials, 203, 73-85.

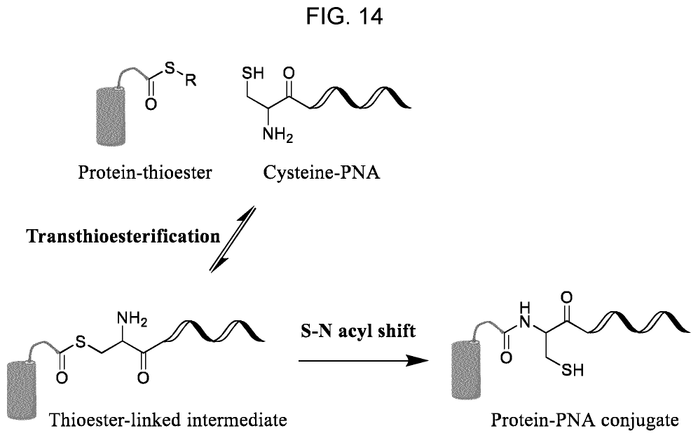

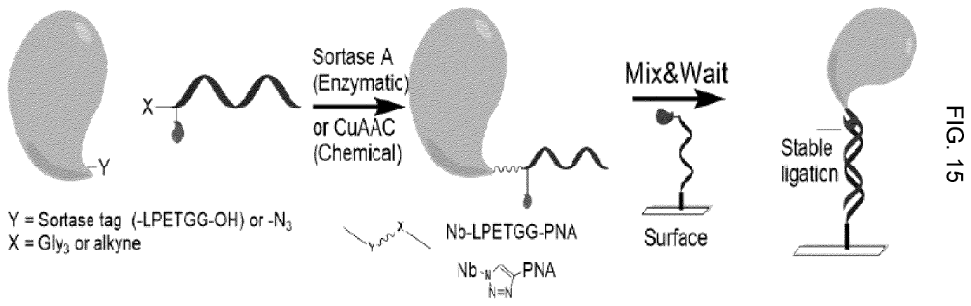

Further, the recognition element may be introduced into the agent by native chemical ligation. For example, an agent such as a protein, a polypeptide or a peptide of interest may comprise a thioester, and the recognition element such as a peptide or PNA to be coupled may comprise a cysteine. By trans-thioesterification and a subsequent S—N acyl shift, the agent and the recognition element may be coupled. This is illustrated in FIG. 14.

For example, the agent may be a small molecule such as biotin and the recognition element may be coupled to the small molecule via a linker such as a PEG linker.

The term “recognition element” refers to a molecule or moiety that has a certain affinity for an other molecule or moiety, e.g. the first recognition element may have an affinity for the second reconition element in the first aspect of the invention, or the first and second recognition element may have an affinity for the third recognition element in the second aspect of the invention. Preferably, the binding between the relevant recognition elements is specific, meaning that two relevant recognition elements bind preferably only to each other and preferably in only one way or orientation. Preferably, the dissociation constant (Kd) between two binding recognition elements is at least at least 2.5 nM, preferably at least 10 nM, at least 100 nM, at least 1 μM, at least 10 μM, or at least 100 μM.

The first recognition element as taught herein and/or the second recognition element as taught herein is a peptide nucleic acid (PNA), a peptide, a peptidomimetic, an oligonucleotide, an oligonucleotide mimic, or a combination thereof.

The term “peptide nucleic acid” or “PNA” refers to an artificially synthesized polymer comprising N-(2-aminoethyl)-glycine (AEG) units linked by peptide bonds. The various purine and pyrimidine bases are linked to the backbone by a methylene bridge (—CH₂—) and a carbonyl group (—(C═O)—). A peptide nucleic acid may further comprise amino acids or amino acid derivatives. PNAs are depicted like peptides, with the N-terminus at the first (left) position and the C-terminus at the last (right) position. PNAs as intended herein may comprise or consist of 3 to 50 units (i.e., N-(2-aminoethyl)-glycine units and amino acid units), preferably 5 to 40 units, such as 6 to 35 units, 7 to 30 units, 8 to 25 units, or 10 to 20 units.

The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol., 13: 132-134).

The term “oligonucleotide” refers to a nucleic acid (including nucleic acid analogues and mimetics) oligomer or polymer as defined herein. Preferably, an oligonucleotide is (substantially) single-stranded. Oligonucleotides as intended herein may be preferably between about 10 and about 100 nucleoside units (i.e., nucleotides or nucleotide analogues) in length, preferably between about 15 and about 50, more preferably between about 20 and about 40, also preferably between about 20 and about 30. Oligonucleotides as intended herein may comprise one or more or all non-naturally occurring heterocyclic bases and/or one or more or all non-naturally occurring sugar groups and/or one or more or all non-naturally occurring inter-nucleoside linkages, the inclusion of which may improve properties such as, for example, increased stability in the presence of nucleases and increased hybridization affinity, increased tolerance for mismatches, etc.

The terms “oligonucleotide mimetic” or “oligomimetic” refer to chemically modified DNA and RNA molecules which exhibit enhanced stability, bioavailability, specificity and/or efficacy. Examples of such oligonucleotide mimic are locked nucleic acid (LNA), 2′-fluor RNA (F-RNA), Phosphorothioate-Modified DNA (PS-DNA), 2-OMe-RNA.

In certain embodiments of the methods as taught herein, the first recognition element may be a peptide nucleic acid (PNA), a peptide, a peptidomimetic, an oligonucleotide, an oligonucleotide mimic, or a combination thereof, preferably wherein the first recognition element is a peptide or PNA, or a combination thereof.

In certain embodiments of the methods as taught herein, the first recognition element may be an oligonucleotide, such as DNA or RNA, or an oligonucleotide mimic.

In certain embodiments of the methods as taught herein, the first recognition element may be an element comprising (a combination of) a peptide nucleic acid and a peptide.

The recitation “element comprising (a combination of) a peptide nucleic acid and a peptide” as used herein refers to an element comprising a continuous sequence of N-(2-aminoethyl)-glycine (AEG) units and amino acid or amino acid derivative units linked by peptide bonds. The element may comprise the AEG units and amino acid or amino acid derivative units in any order. For instance, the element may comprise one or more repeats of a sequence of AEG units and a sequence of amino acid or amino acid derivative units, or the element may comprise a sequence of amino acid or amino acid derivative units followed by a sequence of AEG units followed by a sequence of amino acid or amino acid derivative units, or combinations thereof.

The first recognition element as taught herein comprises an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀ alkenyl, C₆₋₁₅aryl, or C₅₋₁₅heteroaryl group are optionally substituted with an C₁₋₆ alkyl, C₃₋₆ cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.

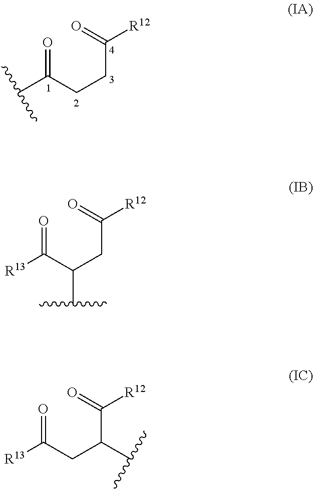

The terms “1,4-dioxo moiety”, “1,4-dioxo-moiety” or “1,4-dioxo group” may be used interchangeably and refers to a moiety or group having two carbonyl functionalities in a 1,4-position relative to each other. In the structure of Formula IA or I, the carbon atoms are numbered to illustrate the 1,4-positioning. The numbering is independent from the IUPAC nomenclature of organic chemistry. The 1,4-positioning (although not indicated by numbering) is also present in the structures of Formula IB and Formula IC. The terms “Formula I” or “Formula IA” can be used interchangeably herein.

The 1,4-dioxo moiety may be coupled to the first recognition element via the carbon atom at position 1 (i.e. 1,4-dioxo moiety having a structure of Formula IA), the carbon atom at position 2 (i.e. 1,4-dioxo moiety having a structure of Formula IB), or the carbon atom at position 3 (i.e. 1,4-dioxo moiety having a structure of Formula IC). Preferably, the 1,4-dioxo moiety may be coupled to the first recognition element via the carbon atom at position 1.

In embodiments, the first recognition element may comprise an 1,4-dioxo moiety having a structure of Formula IA (i.e. of Formula I), wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆ alkyl, C₃₋₆ cycloalkyl, halogen, amine, hydroxyl, sulfhydryl, carboxyl, or C₁₋₆ alkoxy.

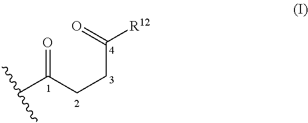

In certain embodiments, the first recognition element as taught herein may comprise a 1,4-dioxo moiety having a structure of Formula I, wherein R¹² is C₁₋₃₀alkyl or C₂₋₃₀alkenyl.

In certain embodiments of the methods or products (such as kits of parts or PNA/peptides) as taught herein, R¹² is C₁₋₃₀alkyl or C₂₋₃₀alkenyl, wherein each of the C₁₋₃₀alkyl or C₂₋₃₀ alkenyl is optionally substituted with an C₁₋₆alkyl, C₃₋₆ cycloalkyl, halogen, amine, hydroxyl, sulfhydryl, carboxyl, or C₁₋₆alkoxy. In certain embodiments of the methods or products (such as kits of parts or PNA/peptides) as taught herein, R¹² is C₁₋₃₀alkyl or C₂₋₃₀alkenyl. In certain embodiments, R¹² is C₁₋₂₀ alkyl or C₂₋₂₀ alkenyl. In certain embodiments, R¹² is C₁₋₁₀alkyl or C₂₋₁₀ alkenyl, preferably R¹² is C₁₋₆alkyl or C₂₋₆ alkenyl, more preferably R¹² is C₁₋₅ alkyl or C₂₋₅alkenyl, still more preferably R¹² is C₁₋₄alkyl or C₂₋₄alkenyl, such as methyl, ethyl, n-propyl, i-propyl, 2-methyl-ethyl, butyl and its isomers (e.g. n-butyl, i-butyl and t-butyl), ethenyl, propenyl, or butenyl.

In certain embodiments of the methods or products (such as kits of parts or PNA/peptides) as taught herein, R¹² is C₁₋₃₀alkyl or C₃₋₃₀alkenyl. In certain embodiments, R¹² is C₁₋₂₀alkyl or C₃₋₂₀alkenyl. In certain embodiments, R¹² is C₁₋₁₀ alkyl or C₃₋₁₀ alkenyl, preferably R¹² is C₁₋₆alkyl or C₃₋₆alkenyl, more preferably R¹² is C₁₋₅ alkyl or C₃₋₅alkenyl, still more preferably R¹² is C₁₋₄alkyl or C₃₋₄alkenyl, such as methyl, ethyl, n-propyl, i-propyl, 2-methyl-ethyl, butyl and its isomers (e.g. n-butyl, i-butyl and t-butyl), propenyl, or butenyl.

In certain embodiments of the methods or products (such as kits of parts or PNA/peptides) as taught herein, R¹² is C₆₋₁₅aryl or C₅₋₁₅heteroaryl, wherein each of the C₆₋₁₅aryl or C₅₋₁₅heteroaryl group is optionally substituted with a C₁₋₆ alkyl, C₃₋₆ cycloalkyl, halogen, amine, hydroxyl, sulfhydryl, carboxyl, or C₁₋₆alkoxy. In certain embodiments, R¹² is C₆₋₁₅aryl, wherein the C₆₋₁₅aryl group is optionally substituted with an C₁₋₆alkyl, C₃₋₆ cycloalkyl, halogen, amine, hydroxyl, sulfhydryl, carboxyl, or C₁₋₆alkoxy. In certain embodiments, R¹² is phenyl, wherein the phenyl group is optionally substituted with an C₁₋₆alkyl, C₃₋₆ cycloalkyl, halogen, amine, hydroxyl, sulfhydryl, carboxyl, or C₁₋₆alkoxy. In certain embodiments, R¹² is phenyl, wherein the phenyl group is optionally substituted with a methyl, chloro, or methoxy. In certain embodiments, R¹² is phenyl or phenyl substituted with a methyl, chloro, or methoxy. For instance, R¹² is phenyl, tolyl, methoxy phenyl, or chlorophenyl, such as R¹² is phenyl, p-tolyl (para-tolyl), o-tolyl (ortho-tolyl), m-tolyl (meta-tolyl), p-methoxy phenyl, o-methoxy phenyl, m-methoxy phenyl, p-chloro phenyl, o-chloro phenyl, or m-chloro phenyl. For instance, R¹² is phenyl, p-tolyl (para-tolyl), p-methoxy phenyl, or p-chloro phenyl.

In certain embodiments of the methods or products as taught herein, R¹² is C₃₋₂₇alkyl or C₁₃₋₂₁ alkenyl. In certain embodiments of the methods or products as taught herein, R¹² is a fatty acid chain.

The term “fatty acid” generally refers to carboxylic acid with a saturated or unsaturated aliphatic chain of carbon atoms. The term “fatty acid chain” refers to a saturated or unsaturated aliphatic chain of carbon atoms. The term “fatty acid chain” includes saturated and unsaturated fatty acid chains. The fatty acid chains or fatty acid moieties may be naturally occurring or synthetic fatty acid chains or fatty acid moieties.

Preferably, R¹² is a methyl group.

In embodiments, R¹³ is hydrogen, C₁₋₃₀alkyl or C₂₋₃₀alkenyl. In certain embodiments, R¹³ is hydrogen, C₁₋₂₀alkyl or C₂₋₂₀alkenyl. In certain embodiments, R¹³ is hydrogen, C₁₋₁₀ alkyl or C₂₋₁₀ alkenyl, preferably R¹³ is hydrogen, C₁₋₆alkyl or C₂₋₆ alkenyl, more preferably R¹³ is hydrogen, C₁₋₅ alkyl or C₂₋₅ alkenyl, still more preferably R¹³ is hydrogen, C₁₋₄alkyl or C₂₋₄alkenyl, such as hydrogen, methyl, ethyl, n-propyl, i-propyl, 2-methyl-ethyl, butyl and its isomers (e.g. n-butyl, i-butyl and t-butyl), ethenyl, propenyl, or butenyl.

In embodiments, R¹³ is hydrogen, C₁₋₃₀alkyl or C₃₋₃₀alkenyl. In certain embodiments, R¹³ is hydrogen, C₁₋₂₀alkyl or C₃₋₂₀ alkenyl. In certain embodiments, R¹³ is hydrogen, C₁₋₁₀alkyl or C₃₋₁₀ alkenyl, preferably R¹³ is hydrogen, C₁₋₆alkyl or C₃₋₆ alkenyl, more preferably R¹³ is hydrogen, C₁₋₅ alkyl or C₃₋₅ alkenyl, still more preferably R¹³ is hydrogen, C₁₋₄alkyl or C₃₋₄ alkenyl, such as hydrogen, methyl, ethyl, n-propyl, i-propyl, 2-methyl-ethyl, butyl and its isomers (e.g. n-butyl, i-butyl and t-butyl), propenyl, or butenyl.

Preferably, R¹³ is hydrogen or methyl, or ethyl. More preferably, R¹³ is hydrogen or methyl.

In embodiments of the methods or products as taught herein, R¹² is methyl, and R¹³, if present, is hydrogen or methyl.

In certain embodiments of the methods or products (such as kits of parts or PNA/peptides) as taught herein, the first recognition element as taught herein may comprises a 1,4-dioxo moiety having a structure of Formula Ia, Ib, Ic, or Id. In certain embodiments of the methods or products as taught herein, the first recognition element as taught herein may comprise a 1,4-dioxo moiety having a structure of Formula Ia.

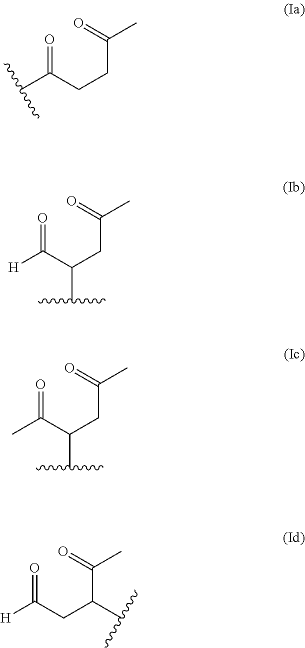

An 1,4-dioxo moiety having a structure of Formula Ia may also be referred to herein as “2,5-dioxopentanyl (DOP) moiety”. The numbering of the positions 2 and 5 in the 2,5-dioxopentanyl moiety is independent from (but corresponds to) the positions 1 and 4 in Formula IA, IB, IC, or I.

Accordingly, an aspect provides a method for covalently binding a first agent and a second agent, the first agent comprising a first recognition element, wherein the first recognition element comprises a 2,5-dioxopentanyl moiety; the second agent comprising a second recognition element, wherein the second recognition element comprises a nucleophilic moiety selected from a hydrazine moiety, an aminooxy moiety, an aminosulfanyl moiety or a hydroxylamine moiety; wherein the first recognition element and the second recognition element are capable of non-covalently binding to each other such that the 2,5-dioxopentanyl moiety and the nucleophilic moiety are brought in proximity; the method comprising contacting the first agent with the second agent, thereby covalently binding the 1,4-dioxo moiety and the nucleophilic moiety.

Likewise, a further aspect provides a method for covalently binding a first agent and a second agent, the first agent comprising a first recognition element, wherein the first recognition element comprises a 2,5-dioxopentanyl moiety; the second agent comprising a second recognition element, wherein the second recognition element comprises a nucleophilic moiety selected from a hydrazine moiety, an aminooxy moiety, an aminosulfanyl moiety or a hydroxylamine moiety; wherein the first recognition element and the second recognition element are capable of non-covalently binding to a third recognition element such that the 2,5-dioxopentanyl moiety and the nucleophilic moiety are brought in proximity; the method comprising contacting the first agent with the second agent and the third recognition element, thereby covalently binding the 1,4-dioxo moiety and the nucleophilic moiety.

In certain embodiments of the methods or products as taught herein, the first recognition element may comprise a 1,4-dioxo moiety having a structure of Formula III or IIIa, wherein:

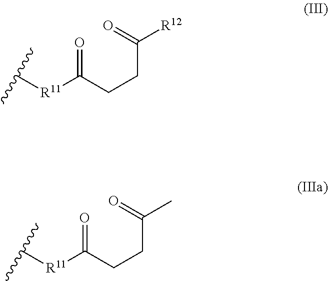

R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy; and

R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy.

In certain embodiments of the methods or products as taught herein, R¹¹ is C₁₋₁₅alkyl, C₃₋₁₅ alkenyl C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy. In certain embodiments, R¹¹ may be methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers.

The 1,4-dioxo moiety may be coupled to the recognition element by any acid stable connection as known in the art such as an ester, an amide, an ether, a triazole, or amine. In embodiments, the 1,4-dioxo moiety may be coupled to the recognition element via the carbon atom at position 1, e.g. as illustrated in structures of Formula III, IIIa, IIIb, IIIc, IIId. Similarly, the 1,4-dioxo moiety may be coupled to the recognition element via the carbon atom at position 2, or the carbon atom at position 3.

In certain embodiments of the methods or products as taught herein, the first recognition element may comprise a 1,4-dioxo moiety having a structure of Formula IIIb, IIIc or IIId, wherein:

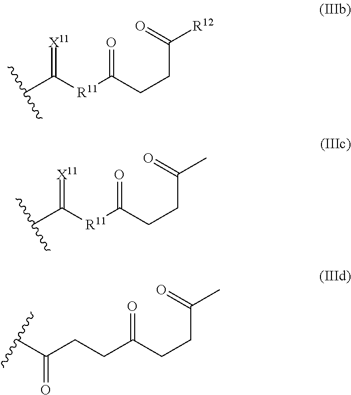

X¹¹ is O, S, or NR¹⁰, wherein R¹⁰ is hydrogen, C₁₋₁₅alkyl or C₆₋₁₅aryl;
R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy; and
R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy.

In certain embodiments, the 1,4-dioxo-moiety may be introduced in the first recognition element as a corresponding furyl moiety having a structure of Formula IIA, IIB, or IIC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀ alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.

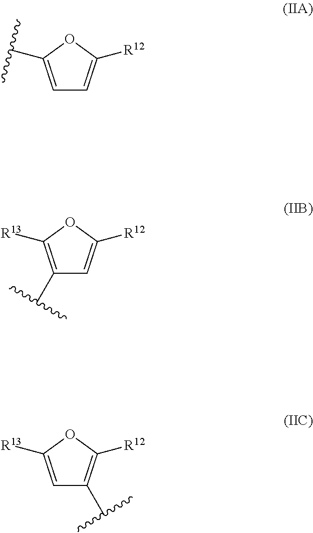

In certain embodiments, the 1,4-dioxo-moiety may be introduced in the first recognition element as a corresponding furyl moiety having a structure of Formula II, wherein R¹² is C₁₋₃₀ alkyl, C₂₋₃₀alkenyl, C₆₋₁₅aryl, or C₅₋₁₅heteroaryl, wherein the C₁₋₃₀ alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy.

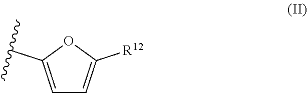

The terms “Formula II” or “Formula IIA” can be used interchangeably herein.

In certain embodiments, the first recognition element may comprise a furyl moiety having a structure of Formula II, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy. In certain embodiments, the first recognition element may comprise a furyl moiety having a structure of Formula II, wherein R¹² is C₁₋₃₀alkyl or C₂₋₃₀alkenyl. In certain embodiments, the first recognition element may comprise an alkyl, alkenyl, aryl, or heteroaryl furyl moiety

The recitations “furyl moiety having a structure of Formula IIA, IIB, IIC or II, wherein R¹² is C₁₋₃₀alkyl” or “alkyl furyl moiety” may be used interchangeably herein.

The recitations “furyl moiety having a structure of Formula IIA, IIB, IIC, or II, wherein R¹² is C₂₋₃₀alkenyl” or “alkenyl furyl moiety” may be used interchangeably herein.

The recitations “furyl moiety having a structure of Formula IIA, IIB, IIC or II, wherein R¹² is C₆₋₁₅aryl” or “aryl furyl moiety” may be used interchangeably herein.

The recitations “furyl moiety having a structure of Formula IIA, IIB, IIC or II, wherein R¹² is C₅₋₁₅heteroaryl” or “heteroaryl furyl moiety” may be used interchangeably herein.

Unless specified otherwise, a furyl moiety having a structure of Formula IIA, IIB, IIC, or II, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀ alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy, is referred to herein as “alkyl, alkenyl, aryl, or heteroaryl furyl moiety” or briefly as “furyl moiety”.

In certain embodiments, the 1,4-dioxo-moiety may be introduced in the first recognition element as an alkyl, alkenyl, aryl, or heteroaryl furyl moiety, followed hydrolysis of the furyl moiety.

In certain embodiments, the alkyl, alkenyl, aryl or heteroaryl furyl moiety may have a structure of Formula IIa, IIb, IIc, IId, IIe, IIf, IIg, IIh, or IIk, wherein X¹¹, R¹¹ and R¹², if present, have the same meaning as that defined herein.

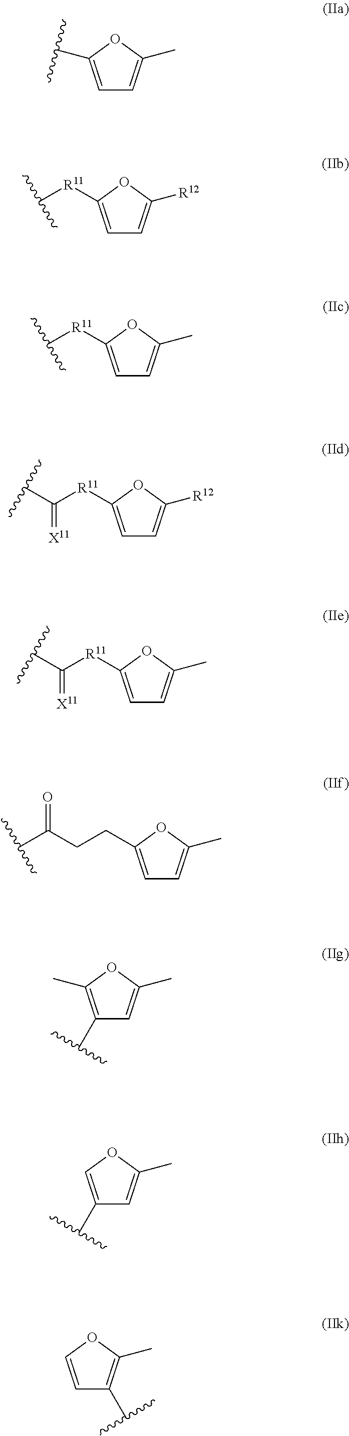

In certain embodiments, the alkyl, alkenyl, aryl, or heteroaryl furyl moiety as taught herein may be coupled to reactive groups in the first recognition element, before the furyl moiety is hydrolysed to the 1,4-dioxo-moiety as taught herein. Classic coupling strategies may be used for such coupling, for example amines from a lysine residue may be used as reactive group in the first coupling element, which can be coupled to a carboxylic acid derivative of the furyl moiety, e.g. 3-(5-methylfuran-2-yl-)propionic acid. For example, HBTU/DIPEA may be used as activating agents. Such coupling reactions may be performed during solid phase synthesis of the first agent or the first recognition element. Appropriate cleavage conditions, such as TFA/m-cresol (9/1) cleavage cocktail, may release the first agent or first recognition element from the solid phase. If present, the protecting groups may be removed, and the furyl moiety may be hydrolysed to the 1,4-dioxo-moieties as taught herein.

Examples of commercially available compounds which can be used for preparing a recognition element comprising an alkyl, alkenyl, aryl, or heteroaryl furyl moiety include 3-(5-methylfuran-2-yl-)propionic acid (ENA358813310, Merck KGaA, Darmstadt, Germany), 2-(2,5-dimethylfuran-3-yl)acetic acid (EN300-2001439, Enamine, NJ, USA), (2,5-dimethylfuran-3-yl)methanamine (ENA413167678, Merck KGaA, Darmstadt, Germany), 4-amino-5-(5-methylfuran-2-yl)pentanoic acid (A02.703.771, Aurora Fine Chemicals LLC, USA), 4-amino-3-(5-methylfuran-2-yl)butanoic acid (EN300-1826531, Enamine, NJ, USA), (3S)-4-amino-3-(5-methylfuran-2-yl)butanoic acid (BBV-56323816, Enamine, NJ, USA), and 2-(aminomethyl)-3-(5-methylfuran-2-yl)propanoic acid (A00.288.775, Aurora Fine Chemicals LLC, USA).

In certain embodiments, the 1,4 dioxo moiety as taught herein can be located at any position of the first recognition element. When the first recognition element is a PNA, a peptide, a peptidomimetic, or a combination thereof, the 1,4-dioxo-moiety may for instance be positioned N-terminally, C-terminally or internally. When the first recognition element is an oligonucleotide, an oligonucleotide mimic, or a combination thereof, the 1,4-dioxo-moiety may for instance be positioned at the 5′-end, 3′-end or internally.

In certain embodiments, the recognition element is a PNA, a peptide, a peptidomimetic, or a combination thereof (including an element comprising a PNA and a peptide), shortened herein to “PNA/peptide”.

In certain embodiments, a PNA/peptide comprising an 1,4-dioxo moiety may be obtained by any suitable method known by the person skilled in the art.

In embodiments, the PNA/peptide as taught herein may be obtained by incorporating a 1,4-dioxo moiety or an alkyl, alkenyl, aryl, or heteroaryl furyl moiety during solid-phase synthesis of a PNA/peptide. Solid-phase synthesis is a method that is widely used to chemically synthesize peptides (see, e.g., Merrifield, 1963, JACS, 85, 2149-2154) and PNAs and can be adapted to produce the PNA/peptide as taught herein. This technique typically comprises two stages: the first stage of solid phase synthesis includes the assembly of a peptide chain using protected amino acid derivatives or the assembly of a PNA chain using protected PNA unit derivatives on a solid support via repeated cycles of coupling-deprotection. The free N-terminal amine of a solid-phase attached PNA/peptide can then be coupled to the C-terminal carboxyl of a single N-protected amino acid unit or PNA unit. This unit is then deprotected, revealing a new N-terminal amine to which a further amino acid may possibly be attached. While the PNA/peptide is being synthesized usually by stepwise methods, all soluble reagents can be removed from the solid support matrix by filtration and washed away at the end of each coupling step. In the second stage, the PNA/peptide is cleaved from the support and side chain protecting groups are removed to produce the PNA/peptide. During the last stage, the alkyl, alkenyl, aryl, or heteroaryl furyl moiety may be hydrolysed to the desired 1,4-dioxo moiety.

In an embodiment, a protected diamino acid may be used for the purpose of introducing an 1,4-dioxo moiety, in particular a 2,5-dioxopentanyl moiety, or the nucleophilic moiety, in a PNA/peptide. In an embodiment, an N-protected amino acid unit such as a lysine amino acid unit can be coupled via solid phase synthesis to a solid phase or growing PNA/peptide chain. The lysine side chain may be modified to a lysine comprising an 1,4-dioxo moiety after deprotection, and reaction with a (5-alkyl, alkenyl, aryl, or heteroaryl furan-2-yl-)carboxylic acid. For instance, Fmoc-Lys_{(Mtt)}-OH or Fmoc-Lys_{(Dde)}-OH can be coupled via solid phase synthesis to a solid phase or growing PNA/peptide chain. The lysine side chain may be modified to a lysine comprising an 1,4-dioxo moiety, in particular a 2,5-dioxopentanyl moiety, by removal of Dde or Mtt, and reaction with 3-(5-methylfuran-2-yl-)propionate. For instance, Ornithine (e.g. Fmoc-L-Orn_{(Mtt)}-OH) may be used for preparing peptides as the recognition element.

In embodiments, the PNA/peptide as taught herein may be obtained by incorporating a 1,4-dioxo-modified amino acid or alkyl, alkenyl, aryl, or heteroaryl furyl-modified amino acid during solid-phase synthesis of a PNA/peptide. Such 1,4-dioxo-modified amino acids, or such alkyl, alkenyl, aryl, or heteroaryl furyl-modified amino acids may be chemically synthesized by methods known in the art.

In certain embodiments, when the first recognition element is a peptide, the peptide may also be obtained by incorporating at least one amino acid comprising an 1,4-dioxo moiety or an alkyl, alkenyl, aryl, or heteroaryl furyl side chain (alkyl, alkenyl, aryl, or heteroaryl furyl amino acid) into a peptide during protein translation in prokaryotes, such as bacteria, e.g. E. coli, or in eukaryotes such as yeast or mammalian cells.

In certain embodiments, the methods as described herein may comprise producing peptides by a method comprising the steps of:
providing a translation system comprising: (i) a 1,4-dioxo-modified amino acid or an alkyl, alkenyl, aryl, or heteroaryl furyl-modified amino acid, (ii) an orthogonal tRNA synthetase, or a functional fragment or variant thereof, (iii) an orthogonal tRNA, wherein said orthogonal tRNA is specifically aminoacylated by said orthogonal tRNA synthetase with the 1,4-dioxo-modified amino acid or the alkyl, alkenyl, aryl, or heteroaryl furyl-modified amino acid, and (iv) a nucleic acid encoding a peptide, wherein the nucleic acid comprises a codon that is recognized by said orthogonal tRNA; and
translating the nucleic acid, thereby incorporating the 1,4-dioxo-modified amino acid or the alkyl, alkenyl, aryl, or heteroaryl furyl-modified amino acid into the peptide.

In certain embodiments, the 1,4-dioxo moiety or the corresponding furyl moiety as taught herein can be located at any position in the PNA/peptide. It will be understood by the skilled person, however, that steric hindrance, e.g. of the furyl moiety, by other amino acids of the PNA/peptide should preferably be avoided. The 1,4-dioxo moiety or the corresponding furyl moiety as taught herein, should preferably be located at a position in the PNA/peptide being accessible for coupling to a second recognition element. The position of the 1,4-dioxo moiety or the corresponding furyl moiety as taught herein in the PNA/peptide is preferably chosen based on, e.g., whether its position in a particular location would change the conformation, activity or stability of the PNA/peptide.

In an embodiment, the PNA/peptide as taught herein may comprise at least three amino acids and/or PNA monomers. Preferably, the PNA/peptide as taught herein may comprise from 3 to 100 amino acids and/or PNA units, for example, the PNA/peptide as taught herein may comprise from 5 to 80 amino acids and/or PNA units or from 6 to 50 amino acids and/or PNA units. For instance, the PNA/peptide as taught herein may contain from 7 to 40 amino acids and/or PNA units from 8 to 30 amino acids and/or PNA units, or from 10 to 20 amino acids and/or PNA units.

In certain embodiments, the first recognition element is an oligonucleotide, an oligonucleotide mimic, or a combination thereof.

In certain embodiments, an oligonucleotide or oligonucleotide mimetic comprising an 1,4-dioxo moiety may be obtained by incorporating an alkyl, alkenyl, aryl, or heteroaryl furyl-modified nucleoside or an alkyl, alkenyl, aryl, or heteroaryl furyl-modified nucleoside-phosphoramidite building block in the oligonucleotide or oligonucleotide mimic and hydrolyzing the alkyl, alkenyl, aryl, or heteroaryl furyl moiety into a 1,4-dioxo moiety. The alkyl, alkenyl, aryl, or heteroaryl furyl-modified nucleoside or alkyl, alkenyl, aryl, or heteroaryl furyl-modified nucleoside-phosphoramidite building block may be incorporated at any position in the polynucleotide sequence. Alkyl, alkenyl, aryl, or heteroaryl furyl-modified nucleosides are known by the person skilled in the art and have been described in literature (e.g. Qian et al., 2015, Helvetica Chimica Acta, 98 (7), 953-960; and Moukha-Chafiq et al., 2019, ACS combinatorial science, 21 (9), 628-634).

In certain embodiments, an oligonucleotide or oligonucleotide mimetic comprising an 1,4-dioxo moiety may be obtained by incorporating an 1,4-dioxo-modified nucleoside or an alkyl, alkenyl, aryl, or heteroaryl furyl-modified nucleoside-phosphoramidite building block (e.g. a 2,5-dioxopentanyl-modified nucleoside) in the oligonucleotide or oligonucleotide mimic. The 1,4-dioxo-modified nucleoside or an alkyl, alkenyl, aryl, or heteroaryl furyl-modified nucleoside-phosphoramidite building block may be incorporated at any position in the polynucleotide sequence.

In certain embodiments of the methods as taught herein, the second recognition element is a peptide nucleic acid (PNA), a peptide, a peptidomimetic, an oligonucleotide, an oligonucleotide mimic, or a combination thereof; preferably the second recognition element is a peptide or PNA, or a combination thereof.

The second recognition element as taught herein comprises a nucleophilic moiety selected from a hydrazine moiety, an aminooxy moiety, an aminosulfanyl moiety or a hydroxylamine moiety.

In certain embodiments of the methods as taught herein, the nucleophilic moiety is a α-effect nucleophile. The term “α-effect nucleophile” refers to a nucleophile wherein nucleophilicity of the nucleophilic centre is increased due to the presence of an adjacent (alpha) atom with lone pair electrons.

The term “hydrazine moiety” as used herein refers to any moiety comprising a nitrogen-nitrogen single covalent bond (i.e., N—N bond) chemically linked to the (remainder of the) second recognition element with one of the nitrogen atoms.

The term “aminooxy moiety” as used herein refers to any moiety comprising an oxygen-nitrogen single covalent bond (i.e., O—N bond) chemically linked to the (remainder of the) second recognition element with the oxygen atom.

The term “aminosulfanyl moiety” as used herein refers to any moiety comprising a sulphur-nitrogen single covalent bond (i.e., S—N bond) chemically linked to the (remainder of the) second recognition element with the sulphur atom.

The term “hydroxylamine moiety” as used herein refers to any moiety comprising a nitrogen-oxygen single covalent bond (i.e., N—O bond) chemically linked to the (remainder of the) second recognition element with the nitrogen atom.

In certain preferred embodiments, the hydrazine moiety, aminooxy moiety, aminosulfanyl moiety or hydroxylamine moiety may be chemically linked to the (remainder of the) second recognition element by binding to (a) carbon atom(s).

In certain embodiments of the methods or products as taught herein, the second recognition element may comprise a nucleophilic moiety having a structure of Formula IV, V, or VI, wherein:

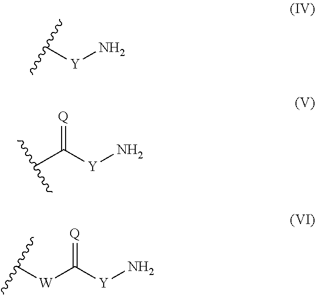

Y is NR¹, O or S, wherein R¹ is hydrogen, C₁₋₃₀alkyl, or C₆₋₂₀ aryl; preferably wherein Y is NR¹, wherein R¹ is hydrogen, C₁₋₃₀alkyl, or C₆₋₂₀ aryl; more preferably wherein Y is NH;
Q is O, S, or NR⁴, wherein R⁴ is hydrogen, C₁₋₃₀alkyl or C₆₋₂₀aryl;
W is NR⁵, O, or S, wherein R⁵ is hydrogen, C₁₋₃₀alkyl or C₆₋₂₀aryl.

In embodiments, Y is NH, Q is O and/or W is NH.

In certain embodiments of the methods as taught herein, the second recognition element may comprise a nucleophilic moiety selected from the group consisting of a hydrazine, a hydrazide, a carbohydrazide, a semicarbazide, a thiosemicarbazide, an iminosemicarbazide, a guanyl hydrazine, a dansyl hydrazine, or a methyl hydrazine.

In certain embodiments of the methods or products as taught herein, the second recognition element may comprise a nucleophilic moiety selected from a hydrazine, a hydrazide or a semicarbazide.

In certain embodiments of the methods or products as taught herein, the second recognition element may comprise a nucleophilic moiety selected from a hydrazine or a semicarbazide. In certain embodiments of the methods or products as taught herein, the second recognition element may comprise a nucleophilic moiety being a hydrazine

In certain embodiments, the second recognition element may comprise a nucleophilic moiety being a hydrazide moiety. In certain embodiments the second recognition element may comprise a nucleophilic moiety being a carbohydrazide. In certain embodiments the second recognition element may comprise a nucleophilic moiety being a semicarbazide moiety or thiosemicarbazide moiety.

The term “hydrazine” as used herein refers to a moiety having the structure —NH—NH₂.

The term “hydrazide” as used herein refers to a moiety having a structure of Formula X.

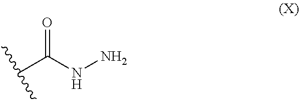 [SMILES: *C(=O)NN]

The term “carbohydrazide” as used herein refers to a moiety having a structure of Formula XI.

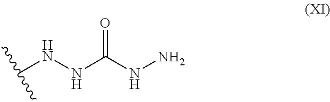 [SMILES: *NNC(=O)NN]

The term “semicarbazide” as used herein refers to a moiety having a structure of Formula XII.

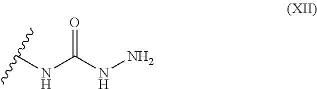 [SMILES: *NC(=O)NN]

The term “thiosemicarbazide” as used herein refers to a moiety having a structure of Formula XIII.

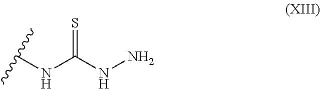 [SMILES: *NC(=S)NN]

The term “iminosemicarbazide” as used herein refers to a moiety having a structure of Formula VIV.

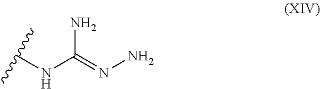 [SMILES: *N/C(N)=N/N]

The term “guanyl hydrazine” as used herein refers to a moiety having a structure of Formula XV.

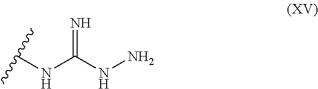 [SMILES: *NC(=N)NN]

The term “dansyl hydrazine” as used herein refers to a moiety having a structure of Formula XVI

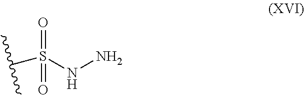 [SMILES: *S(=O)(=O)NN]

The term “methyl hydrazine” as used herein refers to a moiety having the structure —N(CH₃)—NH₂.

In certain embodiments of the methods or products as taught herein, the second recognition element may comprise a nucleophilic moiety having a structure of Formula VII, VIIa, VIII, VIIIa, IX, or IXa, wherein:

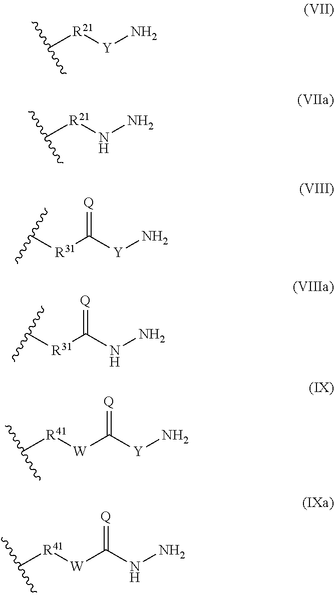

Y is NR¹, O or S, wherein R¹ is hydrogen, C₁₋₃₀alkyl, or C₆₋₂₀aryl;
R²¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
R³¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy;
Q is O, S, or NR⁴, wherein R⁴ is hydrogen, C₁₋₃₀alkyl or C₆₋₂₀aryl;
R⁴¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆ alkoxy;
W is NR⁵, O, or S, wherein R⁵ is hydrogen, C₁₋₃₀alkyl or C₆₋₂₀aryl.

In certain embodiments of the methods or products as taught herein, R²¹, R³¹, R⁴¹ are each independently selected from C₁₋₁₅alkyl, C₃₋₁₅alkenyl C₆₋₁₅aryl, or C₅₋₁₅heteroaryl, wherein the C₁₋₁₅ alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy. In certain embodiments, R²¹, R³¹, R⁴¹ are each independently selected from methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers.

In certain embodiments of the methods or products as taught herein, R³¹ is CR²R³, C₆₋₁₅aryl, or C₅₋₁₅heteroaryl, wherein the C₆₋₁₅ aryl or C₅₋₁₅heteroaryl group are optionally substituted with an C₁₋₆alkyl, C₃₋₆ cycloalkyl, carboxyl, or C₁₋₆alkoxy, wherein R² and R³ are each independently selected from hydrogen, C₁₋₁₅alkyl, C₆₋₁₅aryl, or C₅₋₁₅heteroaryl, wherein the C₁₋₁₅ alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy, with the proviso that only one of R² or R³ is hydrogen.

Preferably, R²¹ is methyl.

In embodiments, R³¹ is C₆aryl, methyl or ethyl. In embodiments, R³¹ is C₆aryl or ethyl. Preferably, R³¹ is C₆aryl.

Preferably, R⁴¹ is methyl.

The nucleophilic moiety may be coupled to the second recognition element by any acid stable connection as known in the art such as an ester, an ether, a triazole, or amine.

In certain embodiments of the methods or products as taught herein, the second recognition element may comprise a nucleophilic moiety having a structure of Formula VIIb, VIIc, or VIId, wherein:

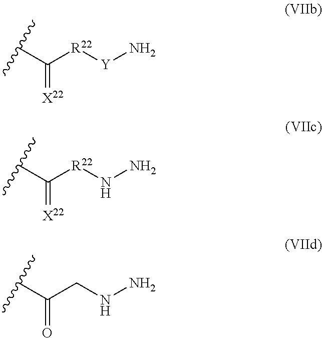

Y is NR¹, O or S, wherein R¹ is hydrogen, C₁₋₃₀alkyl, or C₆₋₂₀aryl;
R²² is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆ alkyl, C₃₋₆cycloalkyl, carboxyl, or C₁₋₆ alkoxy; preferably R²² is methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers;
X²² is O, S, or NR¹⁰, wherein R¹⁰ is hydrogen, C₁₋₁₅alkyl or C₆₋₁₅aryl.

In certain embodiments of the methods or products as taught herein, the second recognition element may comprise a nucleophilic moiety having a structure of Formula VIIIb, VIIIc, VIIId, VIIIe, or VIIIf, wherein:

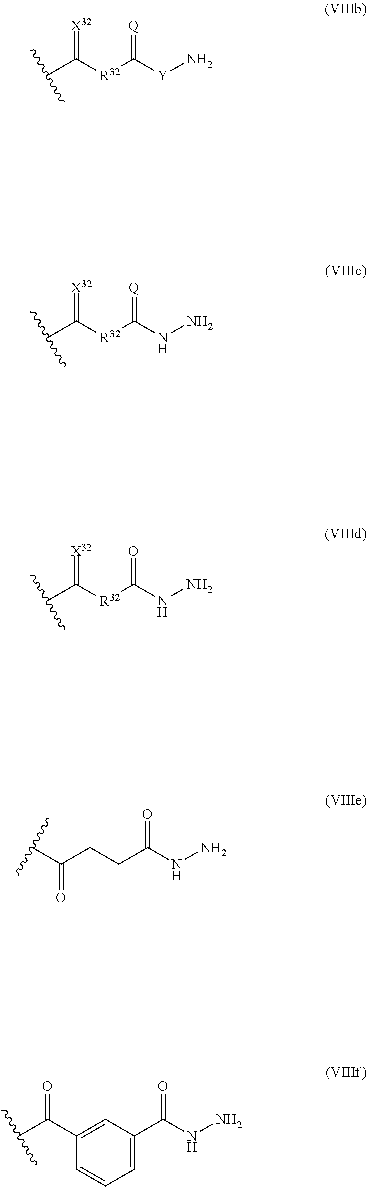

Y is NR¹, O or S, wherein R¹ is hydrogen, C₁₋₃₀alkyl, or C₆₋₂₀aryl;
R³² is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; preferably R²² is methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers;
X³² is O, S, or NR¹⁰, wherein R¹⁰ is hydrogen, C₁₋₁₅alkyl or C₆₋₁₅aryl;
Q is O, S, or NR⁴, wherein R⁴ is hydrogen, C₁₋₃₀alkyl or C₆₋₂₀aryl.

In certain embodiments of the methods or products as taught herein, the second recognition element may comprise a nucleophilic moiety having a structure of Formula IXb, IXc, IXd, or IXe, wherein:

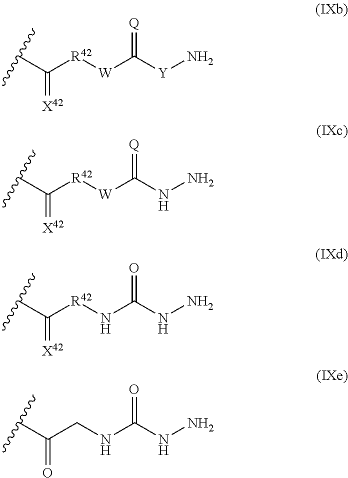

Y is NR¹, O or S, wherein R¹ is hydrogen, C₁₋₃₀alkyl, or C₆₋₂₀aryl;
R⁴² is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; preferably R²² is methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers;
X⁴² is O, S, or NR¹⁰, wherein R¹⁰ is hydrogen, C₁₋₁₅alkyl or C₆₋₁₅aryl;
Q is O, S, or NR⁴, wherein R⁴ is hydrogen, C₁₋₃₀alkyl or C₆₋₂₀aryl;
W is NR⁵, O, or S, wherein R⁵ is hydrogen, C₁₋₃₀alkyl or C₆₋₂₀aryl.

The term “alkyl”, as a group or part of a group, refers to a hydrocarbyl group of Formula CₙH₂ₙ₊₁ wherein n is a number of at least 1. Alkyl groups may be linear or branched and may be substituted as indicated herein. Generally, the alkyl groups comprise from 1 to 30 carbon atoms, preferably from 1 to 15 carbon atoms, more preferably from 1 to 6 carbon atoms, even more preferably 1, 2, 3, 4, 5, or 6 carbon atoms. When a subscript is used herein following a carbon atom, the subscript refers to the number of carbon atoms that the named group may contain.

For example, the term “C₁₋₃₀alkyl”, as a group or part of a group, refers to a hydrocarbyl group of Formula CₙH₂ₙ₊₁ wherein n is a number ranging from 1 to 30. Thus, for example, C₁₋₃₀ alkyl groups include all linear, or branched alkyl groups having 1 to 30 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, nonadecyl and its isomers, icosyl and its isomers, henicosyl and its isomers, docosyl and its isomers, tricosyl and its isomers, tetracosyl and its isomers, pentacosyl and its isomers, hexacosyl and its isomers, heptacosyl and its isomers, octacosyl and its isomers, nonacosyl and its isomers, triacontyl and its isomers.

For example, the term “C₁₋₁₅alkyl”, as a group or part of a group, refers to a hydrocarbyl group of Formula CₙH₂ₙ₊₁ wherein n is a number ranging from 1 to 15. Thus, for example, C₁₋₁₅ alkyl groups include all linear, or branched alkyl groups having 1 to 15 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, and pentadecyl and its isomers.

For example, C₁₋₆alkyl groups include all linear or branched alkyl groups having 1 to 6 carbon atoms, and thus include for example methyl, ethyl, n-propyl, i-propyl, 2-methyl-ethyl, butyl and its isomers (e.g. n-butyl, i-butyl and t-butyl); pentyl and its isomers, and hexyl and its isomers.

The term “C₂₋₃₀alkenyl” as a group or part of a group, refers to an unsaturated hydrocarbyl group, which may be linear, branched or cyclic, comprising one or more carbon-carbon double bonds. Alkenyl groups preferably comprise between 2 and 30 carbon atoms, preferably between 2 and 15 carbon atoms, more preferably between 2 and 6 carbon atoms, such as between 2 and 4 carbon atoms, or between 2 and 3 carbon atoms. Non-limiting examples of alkenyl groups are ethenyl, 2-propenyl, 2-butenyl, 3-butenyl, 2-pentenyl and its isomers, 2-hexenyl and its isomers, 2,4-pentadienyl and the like.

The term “cycloalkyl”, as a group or part of a group, refers to a cyclic alkyl group, that is a monovalent, saturated, hydrocarbyl group having 1 or more cyclic structures, and comprising from 3 to 12 carbon atoms, more preferably from 3 to 9 carbon atoms, more preferably from 3 to 6 carbon atoms, still more preferably from 5 to 6 carbon atoms. Cycloalkyl includes all saturated hydrocarbon groups containing 1 or more rings, including monocyclic or bicyclic groups. The further rings of multi-ring cycloalkyls may be fused, bridged, and/or joined through one or more spiro atoms. The term “C₃₋₆ cycloalkyl”, as used herein, refers to a cyclic alkyl group comprising from 3 to 6 carbon atoms, more preferably from 5 to 6 carbon atoms. Non-limiting examples of C₃₋₆cycloalkyl groups include cyclopropyl, cyclobutyl, cyclopentyl, cyclohexyl. Cycloalkyl groups may also be considered to be a subset of homocyclic rings discussed hereinafter.

The term “homocyclic ring” as a group or part of a group, refers to a ring wherein the ring atoms comprise only carbon atoms. Non limiting examples of homocyclic rings include cycloalkyl, cycloalkenyl, with cycloalkyl being preferred. Where a ring carbon atom is replaced with a heteroatom, preferably nitrogen, oxygen or sulphur, the heteroatom-containing ring resultant from such a replacement is referred to herein as a heterocyclic ring. More than one carbon atom in a ring may be replaced so forming heterocyclic ring having a plurality of heteroatoms.

The term “C₁₋₆alkoxy” or “C₁₋₆alkyloxy”, as a group or part of a group, refers to a group having the Formula —OR^{a} wherein R^{a} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy. Preferably, the C₁₋₆alkoxy is methoxy.

The terms “aryl” or “C₆₋₁₅aryl” as used herein refers to a polyunsaturated, aromatic hydrocarbyl group having a single ring (i.e. phenyl) or multiple aromatic rings fused together (e.g. naphthalene) or linked covalently, typically containing 6 to 15 atoms; preferably 6 to 10, wherein at least one ring is aromatic. The aromatic ring may optionally include one to two additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7- or 8-azulenyl, naphthalen-1- or -2-yl, 4-, 5-, 6 or 7-indenyl, 1-2-, 3-, 4- or 5-acenaphtylenyl, 3-, 4- or 5-acenaphtenyl, 1-, 2-, 3-, 4- or 10-phenanthryl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl. Where a carbon atom in an aryl group is replaced with a heteroatom, the resultant ring is referred to herein as a heteroaryl ring.

The term “heteroaryl” as used herein by itself or as part of another group refers but is not limited to 5 to 20, preferably 5 to 15, carbon-atom aromatic rings or ring systems containing 1 to 2 rings which are fused together or linked covalently, typically containing 5 to 6 atoms; at least one of which is aromatic in which one or more carbon atoms in one or more of these rings can be replaced by oxygen, nitrogen or sulphur atoms where the nitrogen and sulphur heteroatoms may optionally be oxidized and the nitrogen heteroatoms may optionally be quaternized.

In the compounds defined herein, the term “carboxyl” refers to the group —COOH.

In the compounds defined herein, the term “amine” refers to the group —NH₂.

In the compounds defined herein, the term “hydroxyl” refers to the group —OH.

In the compounds defined herein, the term “sulfhydryl” refers to the group —SH.

The term “halo” or “halogen” as a group or part of a group is generic for chloro, fluoro, bromo, iodo. Preferably, the halogen is chloro.

In certain embodiments, the nucleophilic moiety as taught herein can be located at any position of the second recognition element. When the second recognition element is a PNA, a peptide, a peptidomimetic, or a combination thereof, the nucleophilic moiety may for instance be positioned N-terminally, C-terminally or internally. Preferably, the nucleophilic moiety is positioned C-terminally. When the second recognition element is an oligonucleotide, an oligonucleotide mimic, or a combination thereof, the nucleophilic moiety may for instance be positioned at the 5′-end, 3′-end or internally. Preferably, the nucleophilic moiety is positioned at the 3′-end.

In certain embodiments, a PNA/peptide comprising a nucleophilic moiety may be obtained by any suitable method known by the person skilled in the art.

In the methods as taught herein, the nucleophilic moiety, e.g. the hydrazine moiety, the aminooxy moiety, the aminosulfanyl moiety, or the hydroxylamine moiety, may be introduced into or onto the second recognition element by a chemical reaction.

In certain embodiments, a second recognition element comprising a hydrazine moiety may be synthesized or prepared by reacting a second recognition element (e.g., commercially available recognition element) comprising an N-hydroxysuccinimide (NHS) ester with tert-butyl carbazate followed by treatment with trifluoroacetic acid (TFA). For example, a second recognition element comprising a semicarbazide may be prepared by reaction of an NHS derivative of said second recognition element with tert-butyl carbazate followed by treatment with TFA.

In the methods as taught herein, the nucleophilic moiety, e.g. the hydrazine moiety, the aminooxy moiety, the aminosulfanyl moiety, or the hydroxylamine moiety, may be introduced into or onto the second recognition element by solid phase synthesis. In embodiments, the nucleophilic moiety, e.g. the hydrazine moiety, the aminooxy moiety, the aminosulfanyl moiety, or the hydroxylamine moiety, may be introduced at N-terminus of the recognition element for instance using HBTU/DIPEA for activation.

In embodiments, a PNA/peptide comprising a nucleophilic moiety may be obtained via a lysine side chain (provided an orthogonal protecting group is used). For instance, a peptide may be reacted with Tri-Boc-hydrazinoacetic acid or (Boc-aminooxy)acetic acid to introduce a hydrazide moiety on a free amino group.

Alternatively, in certain embodiments, a free N-terminus of a PNA/peptide may be reacted with bis(2,2,2-trifluoroethyl)carbonate or 2,2,2-trifluoroethylchloroformate followed by treatment with a hydrazine hydrate delivering a substituted carbazide (see Bogolubsky et al., 2015, RSC Adv., 5, 1063-1069).

In certain embodiments, a C-terminal hydrazide moiety may be incorporated in a PNA/peptide by starting the synthesis on a Cl-Trt resin, treating it with hydrazine, and subsequently synthesizing the peptide of desired sequence. Final TFA cleavage yields C-terminally modified peptide hydrazides (see Zheng et al., 2013, Nature Protocols, 8, 12, 2483-2495).

In certain embodiments, the second recognition element is an oligonucleotide, an oligonucleotide mimic, or a combination thereof.

In certain embodiments, an oligonucleotide or oligonucleotide mimetic comprising a nucleophilic moiety may be obtained by methods as known in the art such as the method described by Raddatz et al. (Nucleic Acids Research, 2002, 30, 21, 4793-4802).

In certain embodiments, the first agent may be a peptide covalently bound to a first recognition element as taught herein and the second agent may be a peptide covalently bound to a second recognition element as taught herein. The method of the present invention can thus advantageously be used for coupling two peptides to each other.

In certain embodiments, the first agent may be an oligonucleotide covalently bound to a first recognition element as taught herein and the second agent may be a peptide covalently bound to a second recognition element as taught herein. The methods of the present invention therefore allow preparing oligonucleotide-peptide conjugates.

In certain embodiments, the first agent may be a peptide covalently bound to a first recognition element as taught herein and the second agent may be a labeling agent covalently bound to a second recognition element as taught herein. The methods of the present invention thus advantageously allow preparing labeled peptides.

In certain embodiments, the first agent may be a protein covalently bound to a first recognition element as taught herein and the second agent may be a labeling agent covalently bound to a second recognition element as taught herein. The methods of the present invention thus advantageously allow preparing labeled proteins.

In certain embodiments, the first agent may be an oligonucleotide covalently bound to a first recognition element as taught herein and the second agent may be a labeling agent covalently bound to a second recognition element as taught herein. The methods of the present invention thus advantageously allow preparing labeled oligonucleotides.

In certain embodiments of the methods as taught herein, the first agent may be an antibody covalently bound to a first recognition element and the small molecule may be a drug covalently bound to a second recognition element. Such methods advantageously allow the preparation of antibody-drug conjugates whereby the drug is site-specifically coupled to the antibody.

The third recognition element as taught herein is a nucleic acid, an oligonucleotide, an oligonucleotide mimic, a PNA, a protein, a peptide, a cyclodextrin, a cucurbituril, a cyclophane, or a combination thereof.

The term “cyclodextrin” refers to a cyclic oligosaccharide consisting of a macrocyclic ring of glucose subunits joined by α-1,4 glycosidic bonds. Generally, cyclodextrins are composed of 5 or more α-D-glucopyranoside units linked 1 to 4.

The term “cucurbituril” refers to a macrocyclic molecule comprising glycoluril (=C₄H₂N₄O₂═) monomers linked by methylene bridges (—CH₂—). The oxygen atoms are located along the edges of the band and are tilted inwards, forming a partly enclosed cavity.

The term “cyclophane” refers to a hydrocarbon consisting of an aromatic unit (typically a benzene ring) and an aliphatic chain that forms a bridge between two non-adjacent positions of the aromatic ring. More complex derivatives of cyclophanes with multiple aromatic units and bridges forming cage-like structures are also known.

In certain embodiments of the methods or products as taught herein, the first recognition element and the second recognition element may be peptide nucleic acids. In certain embodiments, the first recognition element and the second recognition element may be oligonucleotides. In certain embodiments of the methods or products as taught herein, the first recognition element and the second recognition element may be elements comprising a peptide nucleic acid and a peptide. In certain embodiments of the methods or products as taught herein, the first recognition element and the second recognition element may be peptides. In certain embodiments, the first recognition element may be a PNA and the second recognition element may be an oligonucleotide. In certain embodiments, the first recognition element may be an oligonucleotide and the second recognition element may be a PNA.

In certain embodiments of the methods or products as taught herein, the first recognition element and the second recognition element may be peptide nucleic acids, and the third recognition element may be an oligonucleotide. In certain embodiments, the first recognition element and the second recognition element may be elements comprising a peptide nucleic acid and a peptide, and the third recognition element may be an oligonucleotide.

In embodiments of the methods or products as taught herein, the first recognition element and the second recognition element are capable of non-covalently binding to each other such that the 1,4-dioxo moiety and the nucleophilic moiety are brought in proximity. In embodiments of the methods or products as taught herein, the third recognition element is capable of non-covalently binding to the first recognition element and the second recognition element such that the 1,4-dioxo moiety and the nucleophilic moiety are brought in proximity. The non-covalent binding or non-covalent interaction may be binding by hydrogen bonds, van der Waals interactions, ionic bonds, halogen bonds, or a combination thereof.

The phrases “capable of non-covalently binding to each other”, “capable of non-covalently interacting with each other” or “capable of interacting with each other” may be used interchangeably herein and refer to the capability of molecules (e.g. the recognition elements as taught herein) to interact with each other through one or more variations of electromagnetic interactions, such as hydrogen bonds, van der Waals interactions, ionic bonds, or halogen bonds.

In certain embodiments of the methods or products as taught herein, the first recognition element and the second recognition element may be complementary peptide nucleic acids.

In certain embodiments, the first recognition element and the second recognition element may be complementary oligonucleotides.

In certain embodiments of the methods or products as taught herein, the first recognition element and the second recognition element may comprise complementary peptide nucleic acids. In certain embodiments, the first recognition element and the second recognition element may be elements comprising a peptide nucleic acid and a peptide, wherein the peptide nucleic acid sequences are complementary.

In certain embodiments, the first recognition element may be a PNA and the second recognition element may be an oligonucleotide, wherein the first and the second recognition element are complementary. In certain embodiments, the first recognition element may be an oligonucleotide and the second recognition element may be a PNA, wherein the first and the second recognition element are complementary.

The term “complementarity” as used herein refers to a relationship between two nucleic acid sequences, each sequence being a PNA, a DNA or an RNA sequence, such that when they are aligned antiparallel to each other, the nucleotide bases at each position in the sequences will be complementary, i.e. each nucleotide base pair (A=T, U=T, or G=C) takes up roughly the same space.

Complementarity is achieved by distinct interactions between nucleobases: adenine, thymine (uracil in RNA), guanine and cytosine. In nucleic acid, nucleobases are held together by hydrogen bonding, which only works efficiently between adenine and thymine (uracil in RNA) and between guanine and cytosine. The base complement A=T (A=U in RNA) shares two hydrogen bonds, while the base pair G=C has three hydrogen bonds.

The degree of complementarity between two nucleic acid strands may vary, from complete complementarity (each nucleotide is across from its opposite) to no complementarity (each nucleotide is not across from its opposite) and determines the stability of the sequences to be together. The degree of complementarity (expressed as a percentage) may be calculated by: (the number of complementary nucleobases of two sequences/the number of nucleobases between the first and last complementary nucleobases of two sequences)×100.

In embodiments, the sequence of the first recognition element and the sequence of the second recognition element may have complete complementarity (i.e. 100% complementarity) over a continuous (i.e. noninterrupted) sequence of at least 3 nucleobases. In embodiments, the sequence of the first recognition element and the sequence of the second recognition element may have complete complementarity (i.e. 100% complementarity) over a continuous (i.e. noninterrupted) sequence of at least 5 nucleobases, at least 6 nucleobases, at least 7 nucleobases, at least 8 nucleobases, at least 9 nucleobases, at least 10 nucleobases, at least 11 nucleobases, at least 12 nucleobases, at least 13 nucleobases, at least 14 nucleobases, or at least 15 nucleobases.

In embodiments, the sequence of the first recognition element and the sequence of the second recognition element may have complete complementarity (i.e. 100% complementarity) over a continuous (i.e. noninterrupted) sequence of 3 to 15 nucleobases, such as over a continuous (i.e. noninterrupted) sequence of 5 to 13 nucleobases, over a continuous (i.e. noninterrupted) sequence of 6 to 12 nucleobases, or over a continuous (i.e. noninterrupted) sequence of 7 to 11 nucleobases.

In certain embodiments of the methods or products as taught herein, the first recognition element and the second recognition element are peptide nucleic acids, and the third recognition element is an oligonucleotide, wherein the first recognition element and the second recognition element are complementary to the third recognition element.

In certain embodiments of the methods or products as taught herein, the first recognition element and the second recognition element are oligonucleotides, and the third recognition element is an oligonucleotide, wherein the first recognition element and the second recognition element are complementary to the third recognition element.

In certain embodiments, the first recognition element and the second recognition element are elements comprising a peptide nucleic acid and a peptide, and the third recognition element is an oligonucleotide, wherein the peptide nucleic acid sequences of the first recognition element and second recognition element are complementary to the third recognition element.

In embodiments, the sequence of the first recognition element and the sequence of the third recognition element may have complete complementarity (i.e. 100% complementarity) over a continuous (i.e. noninterrupted) first complementary sequence of at least 3 nucleobases, and the sequence of the second recognition element and the sequence of the third recognition element may have complete complementarity (i.e. 100% complementarity) over a continuous (i.e. noninterrupted) second complementary sequence of at least 3 nucleobases, wherein the first complementary sequence and the second complementary sequence of the third recognition element is (e.g. forms) a continuous (i.e. noninterrupted) sequence or wherein the first complementary sequence and the second complementary sequence of the third recognition element is (e.g. forms) a sequence interrupted by at most 3 nucleotides, such as at most 2 nucleotides or at most 1 nucleotide.

In embodiments, the sequence of the first recognition element and the sequence of the third recognition element may have complete complementarity (i.e. 100% complementarity) over a continuous (i.e. noninterrupted) first complementary sequence of at least 4 nucleobases, at least 5 nucleobases, at least 6 nucleobases, at least 7 nucleobases, at least 8 nucleobases, at least 9 nucleobases, at least 10 nucleobases, at least 11 nucleobases, or at least 12 nucleobases, and the sequence of the second recognition element and the sequence of the third recognition element have complete complementarity (i.e. 100% complementarity) over a continuous (i.e. noninterrupted) second complementary sequence of at least 6 nucleobases, at least 7 nucleobases, at least 8 nucleobases, at least 9 nucleobases, at least 10 nucleobases, at least 11 nucleobases, or at least 12 nucleobases, wherein the first and second complementary sequence of the third recognition element is a continuous (i.e. noninterrupted) sequence, or wherein the first and second complementary sequence of the third recognition element is (e.g. forms) a sequence interrupted by at most 3 nucleotides, such as at most 2 nucleotides or at most 1 nucleotide.

In embodiments, the sequence of the first recognition element and the sequence of the third recognition element may have complete complementarity (i.e. 100% complementarity) over a continuous (i.e. noninterrupted) first complementary sequence of 3 to 15 nucleobases, such as over a continuous (i.e. noninterrupted) sequence of 5 to 13 nucleobases, over a continuous (i.e. noninterrupted) sequence of 6 to 12 nucleobases, or over a continuous (i.e. noninterrupted) sequence of 7 to 11 nucleobases; the sequence of the second recognition element and the sequence of the third recognition element may have complete complementarity (i.e. 100% complementarity) over a continuous (i.e. noninterrupted) second complementary sequence of 3 to 15 nucleobases, such as over a continuous (i.e. noninterrupted) sequence of 5 to 13 nucleobases, over a continuous (i.e. noninterrupted) sequence of 6 to 12 nucleobases, or over a continuous (i.e. noninterrupted) sequence of 7 to 11 nucleobases; and the first and second complementary sequence of the third recognition element is (e.g. forms) a continuous (i.e. noninterrupted) sequence, or wherein the first and second complementary sequence of the third recognition element is (e.g. forms) a sequence interrupted by at most 3 nucleotides, such as at most 2 nucleotides or at most 1 nucleotide.

In certain embodiments of the methods or products as taught herein, the first recognition element and the second recognition element may be interacting coiled coil peptides.

The term “coiled coil” refers to a structural motif in proteins in which at least two alpha-helices are coiled together.

In certain embodiments of the methods or products as taught herein, the third recognition element may be a cyclodextrin, a cucurbituril, a cyclophane, or a combination thereof. These recognition elements form cage-like structures which allow bringing the first recognition element and the second recognition element in proximity.

In certain embodiments, the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the third recognition element is a cyclodextrin, a cucurbituril, a cyclophane, or a combination thereof. In certain embodiments of the methods or products as taught herein, the first recognition element and the second recognition element are peptides, and the third recognition element is a cyclodextrin, a cucurbituril, a cyclophane, or a combination thereof.

In embodiments, an aromatic moiety of the first recognition element and an aromatic moiety of the second recognition element non-covalently interact with a cyclodextrin, a cucurbituril, or a cyclophane. In embodiments, an aromatic moiety of the PNA, peptide, peptidomimetic, oligonucleotide, or oligonucleotide mimic (i.e. first recognition element) and an aromatic moiety of another PNA, peptide, peptidomimetic, oligonucleotide, or oligonucleotide mimic (i.e. second recognition element) non-covalently interact with the cyclodextrin, cucurbituril, or cyclophane. For instance, an aromatic amino side chain of the first peptide (i.e. the first recognition element) and an aromatic amino side chain of the second peptide (i.e. the second recognition element) non-covalently interact with the cyclodextrin, cucurbituril, or cyclophane. Such an aromatic side chain may be for instance the side chain of tryptophan or phenylalanine. Through the non-covalent interaction between an aromatic moiety of the first recognition element and an aromatic moiety of the second recognition element with the cyclodextrin, cucurbituril, or cyclophane advantageously a complex (e.g. 2:1 complex) is formed.

In certain embodiments, the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the third recognition element is a cyclodextrin, a cucurbituril, a cyclophane, or a combination thereof, wherein an aromatic moiety of the first PNA, peptide, peptidomimetic, oligonucleotide, or oligonucleotide mimic and an aromatic moiety of the second PNA, peptide, peptidomimetic, oligonucleotide, or oligonucleotide mimic non-covalently interact with the cyclodextrin, cucurbituril, or cyclophane. In certain embodiments of the methods or products as taught herein, the first recognition element and the second recognition element are peptides, and the third recognition element is a cyclodextrin, a cucurbituril, a cyclophane, or a combination thereof, wherein an aromatic amino acid side chain of the first peptide and an aromatic amino acid side chain of the second peptide non-covalently interact with the cyclodextrin, cucurbituril, or cyclophane.

The term “proximity” refers to a distance between the 1,4-dioxo moiety and the nucleophilic moiety at which covalent bond formation between the 1,4-dioxo moiety and the nucleophilic moiety is induced.

In certain embodiments of the methods as taught herein, the proximity may be the reactive distance between the 1,4-dioxo-moiety and the nucleophilic moiety, meaning the distance at which reaction or covalent bond formation between the 1,4-dioxo moiety and the nucleophilic moiety spontaneously occurs, preferably at physiological conditions, preferably at a temperature of 37° C., preferably at a pH of about 7-8. For instance, the covalent bond between the 1,4-dioxo-moiety and the nucleophilic moiety may be formed in less than 1 hour, such as in less than 30 min, less than 25 min, less than 20 min, less than 15 min, less than 10 min, or in less than 5 min, when the 1,4-dioxo moiety and the nucleophilic moiety are brought in proximity.

It should further be taken into account that the term “proximity induced ligation” is well known in the technical field of the invention. The term “proximity” should be interpreted as the distance at which “proximity induced ligation” may occur.

In embodiments of the methods or products as taught herein, the first recognition element and the second recognition element are capable of non-covalently binding to each other such that the 1,4-dioxo moiety and the nucleophilic moiety are brought within (a distance of) at most 10 Å. For instance, the first recognition element and the second recognition element are capable of non-covalently binding to each other such that the 1,4-dioxo moiety and the nucleophilic moiety are brought within (a distance of) at most 8 Å, at most 6 Å, at most 5 Å, at most 4 Å, or at most 3 Å.

In embodiments of the methods or products as taught herein, the third recognition element is capable of non-covalently binding to the first recognition element and the second recognition element such that the 1,4-dioxo moiety and the nucleophilic moiety are brought within (a distance of) at most 10 Å. For instance, the third recognition element is capable of non-covalently binding to the first recognition element and the second recognition element such that the 1,4-dioxo moiety and the nucleophilic moiety are brought within (a distance of) at most 8 Å, at most 6 Å, at most 5 Å, at most 4 Å, or at most 3 Å.

In certain embodiments of the methods or products as taught herein, the binding of the recognition elements (i.e. first and second recognition elements, or first, second and third recognition elements) may bring the terminal nitrogen atom of the hydrazine moiety, the aminooxy moiety or the aminosulfanyl moiety and the carbon atom at position 1 or 4 of the 1,4-dioxo group, within (a distance of) at most 10 Å. In certain embodiments, the binding of the recognition elements (i.e. first and second recognition elements, or first, second and third recognition elements) may bring the terminal nitrogen atom of the hydrazine moiety, the aminooxy moiety or the aminosulfanyl moiety and the carbon atom at position 1 or 4 of the 1,4-dioxo group, within (a distance of) at most at most 8 Å, for example within (a distance of) at most 6 Å, at most 5 Å, at most 4 Å, or at most 3 Å from each other. Bringing the 1,4-dioxo moiety and the nucleophilic moiety in close proximation increases likelihood of reaction and covalent bond formation. In certain embodiments of the methods or products as taught herein, the binding of the recognition elements (i.e. first and second recognition elements, or first, second and third recognition elements) may bring the terminal oxygen atom of the hydroxylamine moiety and the carbon atom at position 1 or 4 of the 1,4-dioxo group, within (a distance of) at most 10 Å. In certain embodiments, the binding of the recognition elements (i.e. first and second recognition elements, or first second and third recognition elements) may bring the terminal oxygen atom of the hydroxylamine moiety and the carbon atom at position 1 or 4 of the 1,4-dioxo group, within (a distance of) at most at most 8 Å, for example within (a distance of) at most 6 Å, at most 5 Å, at most 4 Å, or at most 3 Å from each other. Overall, this results in an efficient coupling reaction between the first agent and the second agent.

In certain embodiments of the methods or products as taught herein, the first recognition element and the second recognition element may be capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å. For example, the first recognition element and the second recognition element may be capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 8 Å, at most 6 Å, at most 5 Å, at most 4 Å, or at most 3 Å.

In certain embodiments of the methods or products as taught herein, the first recognition element and the second recognition element may be capable of non-covalently binding to each other such that the distance between the 1,4-dioxo moiety and the nucleophilic moiety may be about 3 Å to about 10 Å, such as about 5 Å to about 8 Å. In certain embodiments of the methods or products as taught herein, the first recognition element and the second recognition element may be capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is about 3 Å to about 10 Å, such as about 5 Å to about 8 Å.

In certain embodiments of the methods or products as taught herein, the first recognition element and the second recognition element may be capable of non-covalently binding to the third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å. For example, the first recognition element and the second recognition element may be capable of non-covalently binding to the third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 8 Å, at most 6 Å, at most 5 Å, at most 4 Å, or at most 3 Å.

In certain embodiments of the methods or products as taught herein, the first recognition element and the second recognition element may be capable of non-covalently binding to the third recognition element such that the distance between the 1,4-dioxo moiety and the nucleophilic moiety may be about 3 Å to about 10 Å, such as about 5 Å to about 8 Å. In certain embodiments of the methods or products as taught herein, the first recognition element and the second recognition element may be capable of non-covalently binding to the third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is about 3 Å to about 10 Å, such as about 5 Å to about 8 Å.

In embodiments, the methods as taught herein comprise the step of contacting the first agent with the second agent, thereby covalently binding the 1,4-dioxo moiety and the nucleophilic moiety. In embodiments, the methods as taught herein comprise the step of contacting the first agent and the second agent, thereby covalently binding the 1,4-dioxo moiety of the first agent and the nucleophilic moiety of the second agent. The covalent binding of the 1,4-dioxo moiety of the first agent and the nucleophilic moiety of the second agent allows covalent binding of the first agent and the second agent.

Contacting the first agent as taught herein with the second agent as taught herein allows binding of the first recognition element to the second recognition element, or binding of the first recognition element and the second recognition element to the third recognition element; bringing in proximity (e.g. within at most 10 Å) the 1,4-dioxo moiety and the nucleophilic moiety; and reacting the 1,4-dioxo moiety and the nucleophilic moiety, thereby covalently binding the first agent and the second agent.

In particular, contacting the first agent as taught herein with the second agent as taught herein may comprise:
non-covalent binding of the first recognition element to the second recognition element, or non-covalent binding of the first recognition element and the second recognition element to the third recognition element;
bringing in proximity (e.g. within at most 10 Å):
   (i) the carbon atom at position 1 or 4 of the 1,4-dioxo moiety, and
   (ii) the terminal nitrogen atom of the hydrazine moiety, the aminooxy moiety or the aminosulfanyl moiety, or
   (ii′) the terminal oxygen atom of the hydroxylamine moiety; and/or
reacting the 1,4-dioxo moiety and the nucleophilic moiety, thereby covalently binding the first agent and the second agent.

In embodiments, the methods as taught herein comprise the step of contacting the first agent with the second agent, thereby non-covalently binding the first recognition element to the second recognition element, or non-covalently binding the first recognition element and the second recognition element to the third recognition element. The non-covalent binding may be binding by electrostatic interactions such as hydrogen bonding or ionic interactions, or by van der Waals interactions or by halogen bonding.

In certain embodiments, the first agent may be contacted with the second agent in a molar ratio (mole/mole) of from 100:1 to 1:100. In certain embodiments, the first agent may be contacted with the second agent in a molar ratio (mole/mole) from 50:1 to 1:50, from 25:1 to 1:25, from 20:1 to 1:20, from 10:1 to 1:10, or from 5:1 to 1:5. Preferably, the first agent may be contacted with the second agent in a molar ratio (mole/mole) of 1:1.

In certain embodiments, the first agent may be contacted with the second agent in an equivalent ratio of from 100:1 to 1:100. In certain embodiments, the first agent may be contacted with the second agent in an equivalent ratio from 50:1 to 1:50, from 25:1 to 1:25, from 20:1 to 1:20, from 10:1 to 1:10, or from 5:1 to 1:5. Preferably, the first agent may be contacted with the second agent in an equivalent ratio of 1:1.

In certain embodiments, the first agent, second agent and third agent may be contacted with each other in a molar ratio (mole/mole/mole) of from 100:100:1 to 1:1:100. In certain embodiments, the first agent, second agent and third agent may be contacted with each other in a molar ratio (mole/mole/mole) of from 50:50:1 to 1:1:50, from 25:25:1 to 1:1:25, from 20:20:1 to 1:1:20, from 10:10: to 1:1:10, or from 5:5:1 to 1:1:5. Preferably, the first agent, second agent and third agent may be contacted with each other in a molar ratio (mole/mole/mole) of 1:1:1.

In certain embodiments, the first agent, second agent and third agent may be contacted with each other in an equivalent ratio of from of 100:100:1 to 1:1:100. In certain embodiments, the first agent, second agent and third agent may be contacted with each other in an equivalent ratio from 50:50:1 to 1:1:50, from 25:25:1 to 1:1:25, from 20:20:1 to 1:1:20, from 10:10:1 to 1:1:10, or from 5:5:1 to 1:1:5. Preferably, the first agent, second agent and third agent may be contacted with each other in an equivalent ratio of 1:1:1.

In certain embodiments of the methods as taught herein, the method may comprise the steps of:
providing the first agent and the second agent; and
contacting the first agent with the second agent, thereby covalently binding the 1,4-dioxo moiety and the nucleophilic moiety.

In certain embodiments of the methods as taught herein, the method may comprise the steps of:
providing the first agent, the second agent and the third recognition element; and
contacting the first agent with the second agent and the third recognition element, thereby covalently binding the 1,4-dioxo moiety and the nucleophilic moiety.

The first agent as taught herein and/or the second agent as taught herein may be provided in dried or lyophilized form, such as, for instance a powder of the agent.

The first agent as taught herein and/or the second agent as taught herein may be provided in solution. The first agent as taught herein and/or the second agent as taught herein may be provided in a solvent wherein the agent can be dissolved. In certain embodiments, the solvent is water, dichloromethane (DCM), dimethylformamide (DMF), or N-methyl-2-pyrrolidone (NMP), dimethyl sulfoxide (DMSO), methanol, ethanol, chloroform (CHCl₃), acetonitrile (CH₃CN), or tetrahydrofuran (THF), or a combination of the aforementioned solvents. Preferably, the first agent as taught herein and/or the second agent as taught herein are provided in a solvent comprising or consisting of water.

In certain preferred embodiments, the methods as described herein may comprise providing the first agent as taught herein and/or the second agent as taught herein in an aqueous solution.

The first agent as taught herein and/or the second agent as taught herein may be provided in a composition. In an embodiment, the composition preferably comprises or consists of at least 60%, preferably, at least 65%, 70%, 75%, 80%, 85%, 90%, 95%, 96%, 97%, 98%, 99% or 100% of the first agent as taught herein. For example, the composition comprises from about 60% to about 70% of recognition element, for example, from about 70% to about 80% of the first agent as taught herein, for example, from about 80% to about 90% of the first agent as taught herein, for example, the composition comprises from about 90% to about 100% of the first agent as taught herein. In an embodiment, the composition consists of 100% of substantially pure first agent as taught herein as taught herein. In an embodiment, the composition preferably comprises or consists of at least 60%, preferably, at least 65%, 70%, 75%, 80%, 85%, 90%, 95%, 96%, 97%, 98%, 99% or 100% of the second agent as taught herein. For example, the composition comprises from about 60% to about 70% of recognition element, for example, from about 70% to about 80% of the second agent as taught herein, for example, from about 80% to about 90% of the second agent as taught herein, for example, the composition comprises from about 90% to about 100% of the second agent as taught herein. In an embodiment, the composition consists of 100% of substantially pure second agent as taught herein as taught herein.

The recognition element as taught herein (e.g. the first recognition element or recognition element comprising a 1,4-dioxo moiety as taught herein, the second recognition element or recognition element comprising a nucleophilic moiety as taught herein, or the third recognition element) may be provided in dried or lyophilized form, such as, for instance a powder of the recognition element.

The recognition element as taught herein may be provided in solution. The recognition element as taught herein may be provided in a solvent wherein the recognition element can be dissolved. In certain embodiments, the solvent is water, dichloromethane (DCM), dimethylformamide (DMF), or N-methyl-2-pyrrolidone (NMP), dimethyl sulfoxide (DMSO), methanol, ethanol, chloroform (CHCl₃), acetonitrile (CH₃CN), or tetrahydrofuran (THF), or a combination of the aforementioned solvents. Preferably, the recognition element as taught herein is provided in a solvent comprising or consisting of water.

In certain preferred embodiments, the methods as described herein may comprise providing the recognition element as taught herein in an aqueous solution.

The recognition element as taught herein may be provided in a composition. In an embodiment, the composition preferably comprises or consists of at least 60%, preferably, at least 65%, 70%, 75%, 80%, 85%, 90%, 95%, 96%, 97%, 98%, 99% or 100% of the recognition element as taught herein. For example, the composition comprises from about 60% to about 70% of recognition element, for example, from about 70% to about 80% of the first agent as taught herein, for example, from about 80% to about 90% of the recognition element as taught herein, for example, the composition comprises from about 90% to about 100% of the recognition element as taught herein. In an embodiment, the composition consists of 100% of substantially pure recognition element as taught herein as taught herein.

In embodiments, the methods as taught herein comprise the step of contacting the first agent with the second agent, thereby reacting the 1,4-dioxo moiety and the nucleophilic moiety and covalently binding the first agent and the second agent.

In certain embodiments, the methods as described herein may comprise reacting the 1,4-dioxo moiety with the nucleophilic moiety, thereby covalently binding the first agent and the second agent, preferably site-selectively coupling said first agent to said second agent.

In certain embodiments, the 1,4-dioxo-moiety of the first agent as taught herein reacts with the hydrazine moiety of the second agent as taught herein. Preferably, coupling of a first agent to a second agent occurs between the 1,4-dioxo-moiety of the first agent as taught herein and the hydrazine moiety of the second agent as taught herein. Preferably, coupling of a first agent to a second agent occurs between the 2,5-dioxopentanyl moiety of the first agent as taught herein and the hydrazine moiety of the second agent as taught herein.

In an embodiment, the 1,4-dioxo-moiety of the first agent as taught herein reacts with the aminooxy moiety of the second agent as taught herein. Preferably, coupling of a first agent to a second agent occurs between the 1,4-dioxo-moiety of the first agent as taught herein and the aminooxy moiety of the second agent as taught herein. Preferably, coupling of a first agent to a second agent occurs between the 2,5-dioxopentanyl moiety of the first agent as taught herein and the aminooxy moiety of the second agent as taught herein.

In an embodiment, the 1,4-dioxo-moiety of the first agent as taught herein reacts with the aminosulfanyl moiety of the second agent as taught herein. Preferably, coupling of a first agent to a second agent occurs between the 1,4-dioxo-moiety of the first agent as taught herein and the aminosulfanyl moiety of the second agent as taught herein. Preferably, coupling of a first agent to a second agent occurs between the 2,5-dioxopentanyl moiety of the first agent as taught herein and the aminosulfanyl moiety of the second agent as taught herein.

In an embodiment, the 1,4-dioxo-moiety of the first agent as taught herein reacts with the hydroxylamine moiety of the second agent as taught herein. Preferably, coupling of a first agent to a second agent occurs between the 1,4-dioxo-moiety of the first agent as taught herein and the hydroxylamine moiety of the second agent as taught herein. Preferably, coupling of a first agent to a second agent occurs between the 2,5-dioxopentanyl moiety of the first agent as taught herein and the hydroxylamine moiety of the second agent as taught herein.

In certain embodiments of the methods as taught herein, the first agent may be contacted with the second agent, and optionally the third recognition element, without the addition of an activation signal.

In certain embodiments, the first agent, the second agent, and optionally the third recognition element may be contacted without the addition of an exogenous activation signal. In certain embodiments, the first agent, the second agent, and optionally the third recognition element may be contacted without the addition of an exogenous oxidative reagent. The example section illustrates that the present methods allows chemical crosslinking (covalent binding) of a (modified, i.e., 1,4-dioxo-containing) first agent with a (modified, i.e., nucleophile-containing) second agent without any form of exogenous intervention (i.e. without any chemical activation signal such as NBS, and without any physical activation signal such as UV-light).

In certain embodiments of the methods as taught herein, the first recognition element, the second recognition element, and optionally the third recognition element may be contacted in a concentration of at least 2.5 nM, preferably at least 10 nM, at least 100 nM, at least 1 μM, at least 10 μM, or at least 100 μM.

In certain embodiments of the methods as taught herein, the method may be performed (e.g. the step of contacting the first agent with the second agent may be performed):
in an aqueous solution;
at physiological conditions;
at a temperature ranging from 5 to 50° C., preferably at a temperature ranging from 20 to 40° C.;
at a pH ranging from about 3 to about 11, preferably at a pH ranging from about 4 to about 8;
in the absence of a catalyst (e.g. acid catalysis); and/or
in the absence of a dehydrating agent.

In certain embodiments, the methods as taught herein may be performed in solution. The methods as taught herein may be performed in a solvent in which the first agent and/or second agent can be dissolved. In certain embodiments, the solvent may comprise, consist essentially of, or consist of water, DCM, DMF, NMP, DMSO, methanol, ethanol, chloroform, acetonitrile, THF, or a combination thereof. Preferably, the methods as taught herein are performed in a solvent comprising or consisting of water.

The methods as described herein may be performed in an aqueous solution.

The term “aqueous solution” generally refers to a solution in which the solvent comprises, consists essentially of, or consists of water. In certain embodiments, the aqueous solution comprises at least 0.1% of water. For example, the aqueous solution comprises at least 0.5%, at least 1%, at least 5%, at least 10%, at least 20%, at least 30%, at least 40%, at least 50%, at least 60%, at least 70%, at least 80%, at least 90%, at least 95%, or at least 99% of water. In certain embodiments, the solvent consists of water.

The methods as described herein may be performed in an organic solvent such as DCM, DMF, NMP, DMSO, methanol, ethanol, chloroform, acetonitrile, or THF.

In certain embodiments of the methods as taught herein, the first agent as taught herein may be provided in an aqueous solution. In certain embodiments, the second agent as taught herein may be provided in an aqeuous solution.

In certain embodiments of the methods as taught herein, the first agent as taught herein may be provided in an organic solvent. In certain embodiments, the second agent as taught herein may be provided in an organic solvent.

In certain embodiments, the methods as described herein may be performed in an aqueous solution without the use of organic solvents. In certain embodiments, the methods as described herein may be performed in an aqueous solution without the use of dehydrating agents. In certain embodiments, the methods as described herein may be performed without the use of toxic additives (e.g., copper or aniline) and/or without the use of catalysts (e.g., copper or aniline). In certain embodiments, the methods as described herein may be performed in an aqueous solution without the use of organic solvents and/or without the use of reducing agents and/or without the use of toxic additives and/or without the use of catalysts. Such conditions advantageously offer great potential for the methods as described herein. Furthermore, the first agent-second agent conjugates prepared in an aqueous solution are stable.

In certain embodiments, the methods as described herein may be performed at physiological conditions. Advantageously, such conditions allow the application of the methods as described herein in an intracellular context and in vivo.

In certain embodiments, the methods as described herein may be performed at a temperature ranging from 5 to 50° C. In certain embodiments, the methods as described herein may be performed at a temperature ranging from 10 to 40° C., preferably at a temperature ranging from 15 to 30° C.

In certain embodiments, the methods as described herein may be performed at a pH ranging from about 3 to about 11. In certain embodiments, the methods as described herein may be performed at a pH ranging from about 3 to about 10. In certain embodiments, the methods as described herein may be performed at a pH ranging from about 3 to about 9. In certain embodiments, the methods as described herein may be performed at a pH ranging from about 4 to about 8. In certain embodiments, the methods as described herein may be performed at a near neutral pH.

In certain embodiments, the methods as described herein may be performed at physiological conditions and at a pH ranging from about 3 to about 11. In certain embodiments, the methods as described herein may be performed at physiological conditions and at a pH ranging from about 4 to about 8.

In certain embodiments, the methods as described herein may be performed in an aqueous solution, at physiological conditions, and at near neutral pH. Such conditions allow the application of the methods as described herein in an intracellular context and in vivo.

In certain embodiments of the methods as taught herein, the method may comprise the prior steps of:
providing an agent comprising a first recognition element, wherein the first recognition element comprises a furyl moiety having a structure of Formula IIA, IIB, or IIC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀ alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl; and
hydrolysing the furyl moiety, thereby obtaining the first agent (comprising a 1,4-dioxo moiety).

In certain embodiments of the methods as taught herein, the method may comprise the prior steps of: providing an agent comprising a first recognition element, wherein the first recognition element comprises a furyl moiety having a structure of Formula II, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and hydrolysing the furyl moiety, thereby obtaining the first agent (comprising a 1,4-dioxo moiety).

In embodiments, the methods as taught herein may further comprise the step of identifying the first agent-second agent conjugate. In certain embodiments, the methods as taught herein may further comprise the step of identifying the covalent bond between the first agent and the second agent, i.e., identifying the covalent bond between the 1,4-dioxo moiety and the nucleophilic moiety.

In certain embodiments, the methods as taught herein may further comprise the step of identifying a pyridazinium adduct or a 1,2,5-modified pyrrole adduct (e.g., by NMR spectroscopy). These adducts may be formed after the reaction of the 1,4-dioxo moiety as taught herein and the nucleophilic moiety as taught herein.

Identifying the covalent bond between the 1,4-dioxo moiety and the nucleophilic moiety as taught herein is preferably performed by any adequate technique known to the skilled person for protein analysis, preferably by gel electrophoresis or by protein mass spectrometry (MS) analysis such as liquid chromatography-mass spectrometry (LC-MS), high performance liquid chromatography-mass spectrometry (HPLC-MS), reversed phase high performance liquid chromatography-mass spectrometry (RP HPLC-MS), matrix-assisted laser desorption/ionization-time of flight (MALDI-TOF), electro spray ionization-mass spectrometry (ESI-MS), more preferably by gel electrophoresis, liquid chromatography-mass spectrometry (LC-MS), matrix-assisted laser desorption/ionization-time of flight (MALDI-TOF) or electro spray ionization-mass spectrometry (ESI-MS). These techniques for protein mass spectrometry analysis can optionally be preceded by a tryptic digest. The methods as described herein can thus be easily monitored by analytical techniques.

RP-HPLC is generally used to monitor the reaction progress according to the retention time of the analytes which is related to the polarity of the analytes. Measurement of the absorbance by a UV detector may be helpful in monitoring the different steps of the methods as taught herein.

MALDI-TOF analysis may provide information on the mass of the molecules involved.

Identifying the covalent bond between the 1,4-dioxo-moiety and with the nucleophilic moiety may be performed by nuclear magnetic resonance (NMR) spectroscopy as known in the art. NMR spectroscopy analysis may provide information on the structure and the stereochemistry of the formed species.

A further aspect provides a kit of parts comprising: a) a first recognition element comprising an 1,4-dioxo moiety having a structure of Formula IA, IB or IC, wherein R¹² is C₁₋₃₀alkyl, C₂₋₃₀ alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀ alkyl or C₂₋₃₀alkenyl; b) a second recognition element comprising a hydrazine moiety, an aminooxy moiety, an aminosulfanyl moiety, or a hydroxylamine moiety; wherein the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the first recognition element and the second recognition element are capable of non-covalently binding to each other such that the 1,4-dioxo moiety and the nucleophilic moiety are brought in proximity.

Yet a further aspect provides a kit of parts comprising: a′) a first recognition element comprising an 1,4-dioxo moiety having a structure of Formula IA, IB or IC, wherein R¹² is C₁₋₃₀ alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl; b′) a second recognition element comprising a hydrazine moiety, an aminooxy moiety, an aminosulfanyl moiety, or a hydroxylamine moiety; and c′) a third recognition element capable of non-covalently binding to the first recognition element and the second recognition element such that the 1,4-dioxo moiety and the nucleophilic moiety are brought in proximity; wherein the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the third recognition element is a nucleic acid, an oligonucleotide, an oligonucleotide mimic, a PNA, a protein, a peptide, a cyclodextrin, a cucurbituril, a cyclophane, or a combination thereof.

A further aspect relates to a kit of parts comprising: a) a first recognition element comprising an 1,4-dioxo moiety having a structure of Formula I, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and b) a second recognition element comprising a hydrazine moiety, an aminooxy moiety, an aminosulfanyl moiety, or a hydroxylamine moiety; wherein the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the first recognition element and the second recognition element are capable of non-covalently binding to each other such that the 1,4-dioxo moiety and the nucleophilic moiety are brought in proximity.

A further aspect provides a kit of parts comprising: a′) a first recognition element comprising an 1,4-dioxo moiety having a structure of Formula I, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy; b′) a second recognition element comprising a hydrazine moiety, an aminooxy moiety, an aminosulfanyl moiety, or a hydroxylamine moiety; and c′) a third recognition element capable of non-covalently binding to the first recognition element and the second recognition element such that the 1,4-dioxo moiety and the nucleophilic moiety are brought in proximity; wherein the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the third recognition element is a nucleic acid, an oligonucleotide, an oligonucleotide mimic, a PNA, a protein, a peptide, a cyclodextrin, a cucurbituril, a cyclophane, or a combination thereof.

The terms “kit of parts” and “kit” as used herein refer to a product containing components necessary for carrying out the specified uses or methods, packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified uses or methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the first recognition element as taught herein, the second recognition element as taught herein, and the optional third recognition element as taught herein, the present kits may also include reagents for coupling of the first recognition element to a first agent (which may or may not be included but generally is not included in the kit of parts) and/or coupling of the second recognition element to a second agent (which may or may not be included but generally is not included in the kit of parts). The present kits may further comprise excipients such as solvents useful in the specified uses or methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

In certain embodiments, the first recognition element as taught herein may comprise a 1,4-dioxo moiety having a structure of Formula Ia, Ib, Ic, or Id. In certain embodiments, the first recognition element as taught herein may comprise a 1,4-dioxo moiety having a structure of Formula Ia.

In certain embodiments, the first recognition element as taught herein may comprise a 2,5-dioxopentanyl (DOP) moiety.

Accordingly, a further aspect relates to a kit of parts comprising:
a) a first recognition element comprising an 1,4-dioxo moiety having a structure of Formula Ia, Ib, Ic, or Id, or comprising a 2,5-dioxopentanyl moiety; and
b) a second recognition element comprising a hydrazine moiety, an aminooxy moiety, an aminosulfanyl moiety, or a hydroxylamine moiety;
   wherein the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the first recognition element and the second recognition element are capable of non-covalently binding to each other such that the 1,4-dioxo moiety and the nucleophilic moiety are brought in proximity.

A further aspect provides a kit of parts comprising:
a′) a first recognition element comprising an 1,4-dioxo moiety having a structure of Formula Ia, Ib, Ic, or Id, or comprising a 2,5-dioxopentanyl moiety;
b′) a second recognition element comprising a hydrazine moiety, an aminooxy moiety, an aminosulfanyl moiety, or a hydroxylamine moiety;
c′) a third recognition element capable of non-covalently binding to the first recognition element and the second recognition element such that the 1,4-dioxo moiety and the nucleophilic moiety are brought in proximity; wherein the first recognition element is a PNA, a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the third recognition element is a nucleic acid, an oligonucleotide, an oligonucleotide mimic, a PNA, a protein, a peptide, a cyclodextrin, a cucurbituril, a cyclophane, or a combination thereof.

A further aspect relates to a PNA/peptide comprising an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.

A further aspect relates to a PNA/peptide as taught herein comprising an 1,4-dioxo moiety having a structure of Formula I, wherein R¹² is C₁₋₃₀alkyl, C₂₋₃₀alkenyl, C₆₋₁₅aryl, or C₅₋₁₅ heteroaryl, wherein the C₁₋₃₀alkyl, C₂₋₃₀ alkenyl, C₆₋₁₅aryl, or C₅₋₁₅heteroaryl group are optionally substituted with an C₁₋₆ alkyl, C₃₋₆ cycloalkyl, halogen, amine, hydroxyl, sulfhydryl, carboxyl, or C₁₋₆alkoxy.

Hence, aspects provide:
a peptide nucleic acid (PNA) comprising an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆ alkyl, C₃₋₆ cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.
a peptide comprising an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀ alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.
a peptidomimetic comprising an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.
an element comprising a PNA and a peptide comprising an 1,4-dioxo moiety having a structure of Formula IA, IB or IC, wherein R¹² is C₁₋₃₀alkyl, C₂₋₃₀alkenyl, C₆₋₁₅aryl, or C₅₋₁₅ heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy; and R¹³, if present, is hydrogen, C₁₋₃₀alkyl or C₂₋₃₀ alkenyl.
a peptide nucleic acid (PNA) comprising an 1,4-dioxo moiety having a structure of Formula I, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆alkoxy.
a peptide comprising an 1,4-dioxo moiety having a structure of Formula I, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀ alkenyl, C₆₋₁₅aryl, or C₅₋₁₅heteroaryl group are optionally substituted with an C₁₋₆alkyl, C₃₋₆ cycloalkyl, halogen, amine, hydroxyl, sulfhydryl, carboxyl, or C₁₋₆ alkoxy.
a peptidomimetic comprising an 1,4-dioxo moiety having a structure of Formula I, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy.
an element comprising a PNA and a peptide comprising an 1,4-dioxo moiety having a structure of Formula I, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy.

In certain embodiments, the PNA/peptide as taught herein may comprise a 1,4-dioxo moiety having a structure of Formula Ia, Ib, Ic, or Id.

In certain embodiments, the PNA/peptide as taught herein may comprise a 2,5-dioxopentanyl (DOP) moiety.

Accordingly, further aspects relate to:
a peptide nucleic acid (PNA) comprising an 1,4-dioxo moiety having a structure of Formula Ia, Ib, Ic, or Id, or comprising a 2,5-dioxopentanyl moiety.
a peptide comprising an 1,4-dioxo moiety having a structure of Formula Ia, Ib, Ic, or Id, or comprising a 2,5-dioxopentanyl moiety.
a peptidomimetic comprising an 1,4-dioxo moiety having a structure of Formula Ia, Ib, Ic, or Id, or comprising a 2,5-dioxopentanyl moiety.
an element comprising a PNA and a peptide comprising an 1,4-dioxo moiety having a structure of Formula Ia, Ib, Ic, Id, or comprising a 2,5-dioxopentanyl moiety.

Amino acids with their three-letter code and one letter code are listed in Table 1.

TABLE 1
Amino acids with their three-letter code and one letter code

| Amino acid | Three letter code | One letter code |
|---|---|---|
| glycine | Gly | G |
| alanine | Ala | A |
| valine | Val | V |
| leucine | Leu | L |
| isoleucine | Ile | I |
| proline | Pro | P |
| tyrosine | Tyr | Y |
| tryptophan | Trp | W |
| phenylalanine | Phe | F |
| cysteine | Cys | C |
| methionine | Met | M |
| serine | Ser | S |
| threonine | Thr | T |
| lysine | Lys | K |
| arginine | Arg | R |
| histidine | His | H |
| aspartic acid | Asp | D |
| glutamic acid | Glu | E |
| asparagine | Asn | N |
| glutamine | Gln | Q |

### EXAMPLES

### Example 1: Methods for Covalently Binding a First Agent and a Second Agent According to Embodiments of the Invention

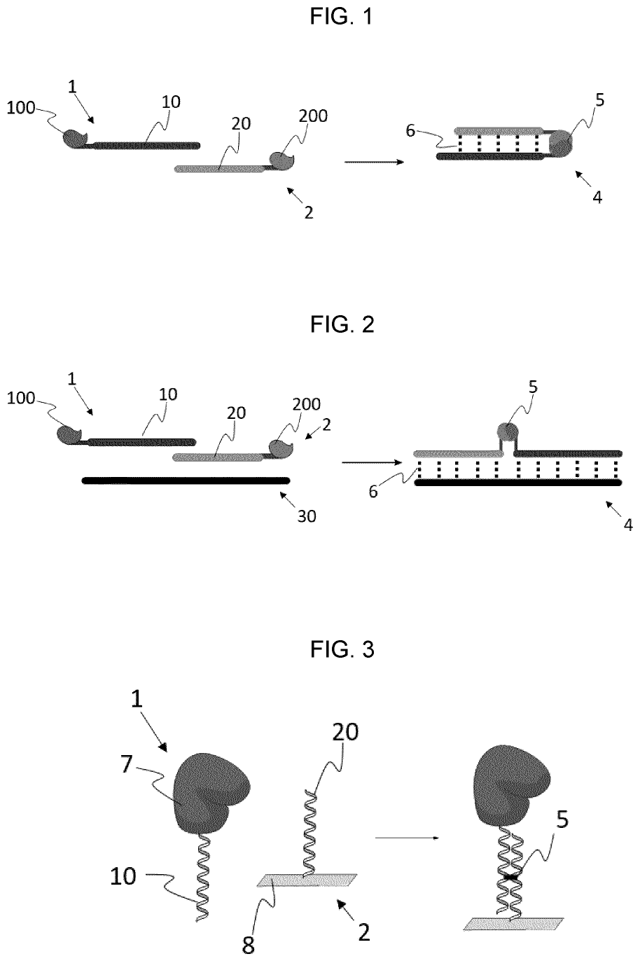

FIG. 1 depicts the principle of a method according to a first embodiment of the invention for covalently binding a first agent (1) and a second agent (2). The first agent (1) comprises a first recognition element (10). The first recognition element comprising a 1,4-dioxo moiety (100) as taught herein. The second agent (2) comprises a second recognition element (20). The second recognition element comprising a nucleophilic moiety (200) as taught herein. The first recognition element (10) is capable of non-covalently binding to the second recognition element (20), thereby forming an adduct (4) and bringing the 1,4-dioxo moiety (100) of the first recognition element and the nucleophilic moiety (200) of the second recognition element in proximity. The proximity induces the reaction between the 1,4-dioxo moiety and the nucleophilic moiety, thereby forming a covalent bond (5) between the first agent and the second agent. For example, the first recognition element is a peptide nucleic acid, the second recognition element is a peptide nucleic acid, and the oligonucleotide sequence of the first recognition element and the oligonucelotide sequence of the second recognition element are complementary (6) to each other.

FIG. 2 depicts the principle of a method according to a second embodiment of the invention for covalently binding a first agent (1) and a second agent (2) using a third recognition element (30). The first agent (1) comprises a first recognition element (10). The first recognition element comprising a 1,4-dioxo moiety (100) as taught herein. The second agent (2) comprises a second recognition element (20). The second recognition element comprising a nucleophilic moiety (200) as taught herein. The first recognition element (10) is capable of non-covalently binding to the third recognition element (30) and the second recognition element (20) is also capable of non-covalently binding to the third recognition element (30), thereby forming an adduct (4) and bringing the 1,4-dioxo moiety (100) and the nucleophilic moiety (200) in proximity. The proximity induces the reaction between the 1,4-dioxo moiety and the nucleophilic moiety, thereby allowing the formation of a covalent bond (5) between the first agent and the second agent. For example, the first recognition element is a peptide nucleic acid, the second recognition element is a peptide nucleic acid, the third recognition element is an oligonucleotide, and the oligonucleotide sequence of the first recognition element and the oligonucelotide sequence of the second recognition element are each complementary (6) to a part of the sequence of the third reconition element.

FIG. 3 depicts the principle of a method according to an embodiment of the invention for preparing a protein array. The first agent (1) is a protein (7) covalently bound to a first recognition element (10), such as a PNA, comprising a 1,4-dioxo moiety (not shown) as taught herein. The second agent (2) is a surface (8), for example the bottom surface of a well in a 96-well plate, covalently bound to a second recognition element (20), such as a PNA. The second recognition element (20) comprise a nucleophilic moiety (not shown) as taught herein. Upon contacting the first agent (1) with the second agent (2), the recognition elements (10, 20) bind to each other, inducing the formation of a covalent bond (5) between the 1,4-dioxo moiety and the nucleophilic moiety.

Materials and Methods

Products

All reagents were purchased from Sigma-Aldrich, Fluka, Merck, TCI Europe, fluorochem and used without further purification. Dry DMF was stored over 4 Å molecular sieves. DNA and RNA sequences were purchased from IDT (Leuven, Belgium).

Analyses

NMR spectra were recorded on a Brucker Avance 300 or 400. δ values are expressed in ppm relatively either to CDCl₃ (7.29 ppm for proton and 76.9 ppm for carbon) or DMSO-d⁶ (2.50 ppm for proton and 39.5 ppm for carbon). The following abbreviations are used to explain the multiplicities: s=singlet, d=doublet, t=triplet, q=quartet, m=multiplet, and br=broad.

HPLC-MS data were collected on an Agilent 1100 Series instrument equipped with a Phenomenex Kinetex C18 100 Å column (150×4.6 mm, 5 μm at 35° C.) connected to an ESMSD type VL mass detector (quadrupole ion trap mass spectrometer) with a flow rate of 1.5 ml/min was used with the following solvent systems: (A): 0.1% HCOOH in H₂O and (B) MeCN. Gradient: 100% A for 2 min, then a gradient from 0 to 100% B over 6 min was used, followed by 2 min of flushing with 100% B (HPLC1) or 100% A for 0.5 min, a gradient from 0 to 10% B over 0.1 min and then from 10% to 30% B over 7.7 minutes was used, followed by 2 min of flushing with 100% B (HPLC2).

HPLC-UV data were collected on an Agilent 1100 Series instrument equipped with a Waters XTERRA RP18 5 μm column (250×2.1 mm at 40° C. or 50° C.) connected to a DAD using a flow rate of 0.35 ml/min with the following solvent systems: (A): 0.1% TFA in H₂O and (B) 0.1% TFA in MeCN. Gradient: 100% A for 1 min, then a gradient from 0 to 10% B in 1 min, then to 30% B in 10 min, and finally to 100% B in 1 min, followed by 3.5 min of flushing with 100% B (HPLC3); 100% A for 1 min, then a gradient from 0 to 30% B in 1 min, then to 60% B in 10 min, and finally to 100% B in 1 min, followed by 3.5 min of flushing with 100% B (HPLC4). PNA oligomers were purified using a Phenomenex Luna C18(2) (5 μm, 100 Å, 250×4.6 mm) (HPLC5, 100% A for 5 min, then a gradient from 0 to 50% B over 30 min at a flow rate of 4.0 ml/min). Peptide oligomers were purified using a Phenomenex Luna C18(2) (5 μm, 250 Å, 250×21.2 mm) on an Agilent 218 solvent delivery system (HPLC6, 100% A for 2 min, then a gradient to 100% in 30 min at a flow rate of 17.5 ml/min).

UV-VIS spectra were recorded using a Trinean DropSense96 UV/VIS droplet reader.

Thermal denaturation experiments were recorded on a Varian Cary 300 Bio instrument equipped with a six-cell thermostated cell holder.

USDS-PAGE analysis were performed using 15% polyacrylamide gels (5% crosslink, 19:1 acrylamide/bisacrylamide) prepared in Tris-Acetate buffer (50 mM Tris-Acetate, pH 7.6) containing 7 M urea and 0.1% SDS. The temperature of the gel was stabilized with a Julabo F12 at 25° C. The power supply used for gel electrophoresis was a consort EV202 and a voltage of 200 V for 0.75 mm thickness or 100 V for 1.0 mm thickness was used to run the gels (15 minutes pre-run). 2 μL of sample solution were mixed with 3 μL formamide and 5 μL loading buffer (100 mM Tris-Acetate pH 7.6, 7 M urea, 20% formamide, 2% SDS) from this mixture 5/8 μL were loaded on the gel. Gels were stained with Pierce Silver Stain (Thermo Fisher Scientific) and pictures were scanned with an HP Photosmart B110.

Surface ligation experiments were performed using Stuart SSM1 mini orbital shaker for uniform shaking of the wells. The temperature control was ensured by a homemade thermostat incubation chamber.

Microarray slides were scanned with an Agilent G2565CA.

PNA Synthesis

The synthesis of the PNA probes was performed with standard manual Fmoc-based solid-phase synthesis using HBTU/DIPEA as coupling mixture, using commercially available Fmoc-PNA-OH monomers (Biosearch Technologies, Scotland). Rinkamide-ChemMatrix resin was first loaded with Fmoc-Arg_{(pbf)}-OH, Fmoc-Lys_{(Mtt)}-OH or Fmoc-Lys_{(Dde)}-OH as first monomer (0.2 mmol/g). Modification of lysine side chains where performed after Dde deprotection (for a 5 μmol scale: shake vigorously the resin for 1 h in a solution containing 250 mg hydroxylamine hydrochloride and 184 mg imidazole in 1.2 mL NMP/DMF 5:1) or Mtt deprotection (0.5% BtOH-H₂O in HFIP/DCM 1:1, Mtt deprotection can be visually followed by adding 1 drop of TFA to the deprotection solution) removal for 3-(5-methylfuran-2-yl-)propionate and TAMRA coupling, respectively. All coupling steps are performed using HBTU/DIPEA as activating. Cleavage was performed using a TFA/m-cresol (9:1) cleavage cocktail. After RP-HPLC purification (HPLC5), the purity and identity of the PNAs were evaluated by LC-MS (HPLC1).

Peptide Synthesis

The synthesis of the peptide probes was performed with standard manual Fmoc-based solid-phase synthesis using HBTU/DIPEA as coupling mixture, on a Syro automatic peptide synthesizer, using Fmoc-Orn_{(Mtt)}-OH together with standard Fmoc-protected amino acids. Rinkamide-AM Champion resin was directly loaded in the synthesis reactor (0.69 mmol/g). Modification of the ornithine side chain was performed after Mtt removal (0.5% BtOH-H₂O in HFIP/DCM 1:1, Mtt deprotection can be visually followed by adding 1 drop of TFA to the deprotection solution) for 3-(5-methylfuran-2-yl-)propionic acid or tri-Boc-hydrazinoacetic acid coupling. All coupling steps are performed using HBTU/DIPEA as activating mixture. Cleavage was performed using a TFA/m-cresol (9:1) cleavage cocktail. After RP-HPLC purification (HPLC6), the purity and identity of the peptides were evaluated by LC-MS (HPLC1).

TABLE 2
Sequences of peptide nucleic acids or peptides. Capital letters indicate PNA
monomers, small letters indicate L-amino acids, modifications on the lysine
or ornithine side chains are inserted inside brackets. DOP: 4,7-dioxooctanoyl;
X1: (4,7-dioxo-7-phenyl-heptanoyl); X2: [7-(4-methoxyphenyl)-4,7-dioxo-heptanoyl];
X3: [4,7-dioxo-7-(p-tolyl)heptanoyl]; X4: [7-(4-chlorophenyl)-4,7-dioxo-heptanoyl];
X5: (4-acetyl-3-methyl-6-oxo-heptanoyl); O: 2-(2-aminoethoxy)ethoxyacetyl
(AEEA) spacer; ABA: 4-Acetamidobenzoyl; TAMRA: 5-carboxytetramethylrhodamine;
*: 1: first recognition element,
2: second recognition element; **: underlined sequence

| PNA/peptide | Sequence | * | MW | SEQ ID NO: |
|---|---|---|---|---|
| PNA-M1 | Ac-ATGATCT-k(DOP)rr-NH₂ | 1 | 2545.6 | 1 |
| PNA-M2 | Ac-ATCATGT -k(DOP)rr-NH₂ | 1 | 2545.6 | 2 |
| PNA-M3 | Ac-TTATCAG-k(DOP)rr-NH₂ | 1 | 2545.6 | 3 |
| PNA-M4 | H-OO-GTCTTGAGCAG-K(DOP)rr-NH₂ | 1 | 3919.0 | 4 |
| PNA-A1 | H-ß-Ala-k(Ac)-AGATCATGCCC-rrr-NH₂ | 2 | 3672.8 | 5 |
| PNA-C1 | Ac-ß-Ala-k(Ac)-AGATCATGCCC-r3-NH₂ | 2 | 3714.8 | 6 |
| PNA-H1 | H₂N-g-k(Ac)-AGATCATGCCC-rrr-NH₂ | 2 | 3673.8 | 7 |
| PNA-Z1 | H₂N-NHCO(CH₂)₂CO-k(Ac)-AGATCATGCCC-rrr-NH₂ | 2 | 3715.9 | 8 |
| PNA-S1 | H₂N-NHCONH-CH₂CO-k(Ac)-AGATCATGCCC-rrr-NH₂ | 2 | 3716.8 | 9 |
| PNA-A2 | Biot-PEG₄-k(H-ß-Ala-k(TAMRA)-AGATCATGCCC-rrr->)-NH₂ | 2 | 4644.9 | 10** |
| PNA-H2 | Biot-PEG₄-k(H₂N-g-k(TAMRA)-AGATCATGCCC-rrr-->)-NH₂ | 2 | 4687.0 | 11** |
| PNA-C2 | Biot-PEG₄-k(Ac-ß-Ala-k(TAMRA)-AGATCATGCCC-rrr-->)-NH₂ | 2 | 4645.9 | 12** |
| 6-DOP-coil | ABA-eiaal-Orn(DOP)-keiaalekeiaalek-NH₂ | 1 | 2582.98 | 13 |
| 1-Hy-coil | ABA-Orn(H₂N-g-)-iaalkekiaalkekiaalke-NH₂ | 2 | 2498.02 | 14 |
| 6-Hy-coil | ABA-kiaal-Orn(H₂N-g-)-ekiaalkekiaalke-NH₂ | 2 | 2498.02 | 15 |
| 1-Hy-coil(R) | ABA-Orn(H₂N-g-)-iaalreriaalreriaalre-NH₂ | 2 | 2638.12 | 16 |
| 6-Hy-coil(R) | ABA-riaal-Orn(H₂N-g-)-eriaalreriaalre-NH₂ | 2 | 2638.12 | 17 |
| Hy-random | H-Orn(H₂N-g-)-fgydaky-NH₂ | 1 | 1048.16 | 18 |
| 6-X1-coil | ABA-eiaal-Orn(X1)-keiaalekeiaalek-NH₂ | 1 | | 32 |
| 6-X2-coil | ABA-eiaal-Orn(X2)-keiaalekeiaalek-NH₂ | 1 | | 33 |
| 6-X3-coil | ABA-eiaal-Orn(X3)-keiaalekeiaalek-NH₂ | 1 | | 34 |
| 6-X4-coil | ABA-eiaal-Orn(X4)-keiaalekeiaalek-NH₂ | 1 | | 35 |
| 6-X5-coil | ABA-eiaal-Orn(X5)-keiaalekeiaalek-NH₂ | 1 | | 36 |

Characterisation of Synthesized Recognition Elements

The following discloses the yield of each PNA (in %), the retention time (tᵣ) for the obtained recognition element in HPLC chromatograms, the molar extinction coefficient (ε) at 260 nm for the obtained recognition element, the MS spectra for the obtained recognition element.

PNA-M1: 8.5%; tᵣ: 3.08 min (HPLC1); ε=71500 M⁻¹ cm⁻¹; ESI-MS: m/z calcd 2545.6 [M]: 1273.3 [M+2H]²⁺, 849.2 [M+3H]³⁺, 637.2 [M+4H]⁴⁺, 510.0 [M+5H]⁵⁺; PNA-M2: 10.0%; tᵣ: 3.13 min (HPLC1); ε=71500 M⁻¹ cm⁻¹; ESI-MS: m/z calcd 2545.6 [M]: 1273.3 [M+2H]²⁺, 849.2 [M+3H]³⁺, 637.2 [M+4H]⁴⁺, 510.0 [M+5H]⁵⁺, 422.2 [M+6H]⁶⁺; PNA-M3: 8.6%; tᵣ: 3.13 min (HPLC1); ε=71500 M⁻¹ cm⁻¹; ESI-MS: m/z calcd 2545.6 [M]: 1273.3 [M+2H]²⁺, 849.2 [M+3H]³⁺, 637.2 [M+4H]⁴⁺, 510.0 [M+5H]⁵⁺; PNA-M4: 7.1%; tᵣ: 3.09 min (HPLC1); ε=113200 M⁻¹ cm⁻¹; ESI-MS: m/z calcd 3918.9 [M]: 1306.9 [M+3H]³⁺, 980.5 [M+4H]⁴⁺, 784.7 [M+5H]⁵⁺, 654.0 [M+6H]⁶⁺, 560.8 [M+7H]⁷+, 490.8 [M+8H]³⁺; PNA-K1: 10.2%; tᵣ: 2.82 min (HPLC1); ε=108100 M⁻¹ cm⁻¹; ESI-MS: m/z calcd 3559.6 [M]: 1187.1 [M+3H]³⁺, 890.5 [M+4H]⁴⁺, 712.7 [M+5H]⁵⁺, 594.2 [M+6H]⁶⁺, 509.4 [M+7H]⁷+, 446.0 [M+8H]⁸+, 396.5 [M+9H]⁹+; 6-DOP-Coil: 19.9%; tᵣ: 4.69 min (HPLC1); ε=17989 M⁻¹ cm⁻¹ at 270 nm; ESI-MS: m/z calcd 2583.0 [M]: 1291.5 [M+2H]²⁺, 861.3 [M+3H]³⁺, 646.3 [M+4H]⁴⁺; 1-Hy-Coil: 16.6%; tᵣ: 3.81 min (HPLC1); ε=17989 M⁻¹ cm⁻¹ at 270 nm; ESI-MS: m/z calcd 2498.02 [M]: 1249.5 [M+2H]²⁺, 833.4 [M+3H]³⁺, 625.3 [M+4H]⁴⁺, 500.5 [M+5H]⁵⁺, 417.3 [M+6H]⁶⁺; 6-Hy-Coil: 28.4%; tᵣ: 3.82 min (HPLC1); ε=17989 M⁻¹ cm⁻¹ at 270 nm; ESI-MS: m/z calcd 2498.02 [M]: 1249.5 [M+2H]²⁺, 833.4 [M+3H]³⁺, 625.3 [M+4H]⁴⁺, 500.5 [M+5H]⁵⁺, 417.3 [M+6H]⁶⁺; 1-Hy- Coil(R): 7.4%; tᵣ: 3.84 min (HPLC1); ε=17989 M⁻¹ cm⁻¹ at 270 nm; ESI-MS: m/z calcd 2638.1 [M]: 1319.6 [M+2H]²⁺, 880.0 [M+3H]³⁺, 660.3 [M+4H]⁴⁺, 528.5 [M+5H]⁵⁺, 440.5 [M+6H]⁶⁺; 6-DOP-Coil(R): 11.2%; tᵣ: 3.85 min (HPLC1); ε=17989 M⁻¹ cm⁻¹ at 270 nm; ESI-MS: m/z calcd 2638.1 [M]: 1319.6 [M+2H]²⁺, 880.0 [M+3H]³⁺, 660.3 [M+4H]⁴⁺, 528.5 [M+5H]⁵⁺, 440.5 [M+6H]⁶⁺; Hy-Random: 48.5%; tᵣ: 3.15 min (HPLC1); ε=2560 M⁻¹ cm⁻¹ at 280 nm; ESI-MS: m/z calcd 1048.2 [M]: 1049.0 [M+H]+, 524.7 [M+2H]²⁺, 350.3 [M+3H]³⁺;

### Example 2: Methods for Covalently Binding a First Recognition Element and a Second Recognition Element According to Embodiments of the Invention

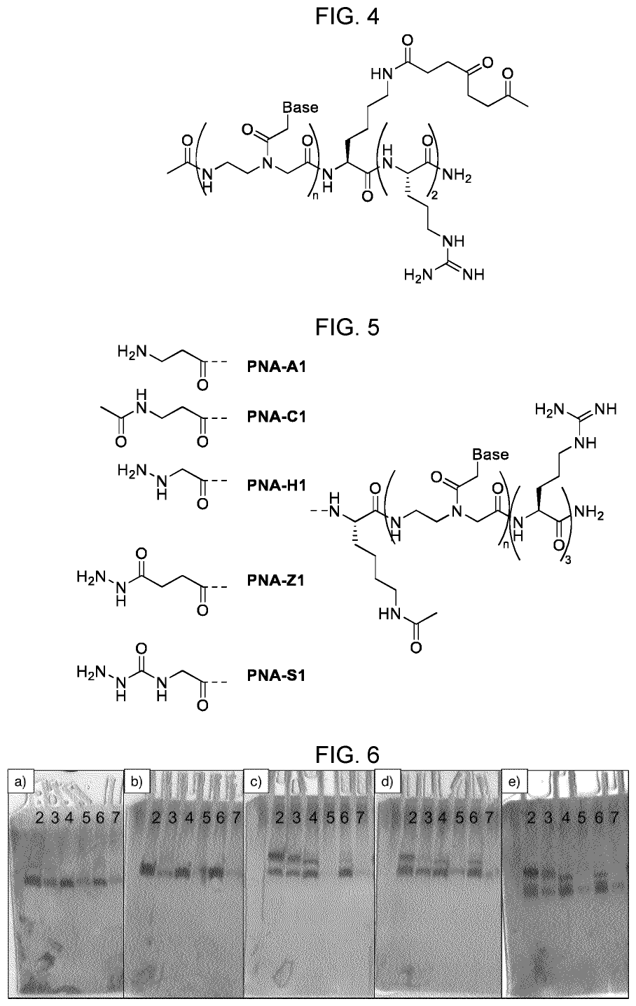

In order to investigate the chemo-selectivity and the nature of the ligation between the 1,4-dioxo moiety and the nucleophilic moiety, first recognition elements comprising a 1,4-dioxo moiety were reacted with second recognition elements comprising different moieties. Three PNAs comprising a 1,4-dioxo moiety were synthesized: fully matched (FM) PNA (PNA-M1), mismatched (MM) PNA (PNA-M2) and scrambled (scr) PNA (PNA-M3) (Table 2 and FIG. 4). The behaviour of these PNAs was tested in the presence of PNAs comprising different nucleophilic moieties: amine-modified PNA (PNA-A1), amide-modified PNA (PNA-C1), hydrazine-modified PNA (PNA-H1), hydrazide-modified PNA (PNA-Z1) and semicarbazide-modified PNA (PNA-S1) (Table 2 and FIG. 5).

In a typical experiment, 100 μL of buffered solutions (PBS, pH 7.4) containing the first agent (i.e. the first recognition element) at 5 μM concentration (from a 100 μM stock solution) and the second agent (i.e. the second recognition element) at 5 μM concentration (from a 100 μM stock solution), were prepared in a 0.5 mL Eppendorf and allowed to react overnight at 25° C. The solutions were collected in the morning, eventually quenched by addition of 50 eq. methylhydrazine (from a 5 mM stock solution), and analysed via HPLC-UV, HPLC-MS and USDS-PAGE.

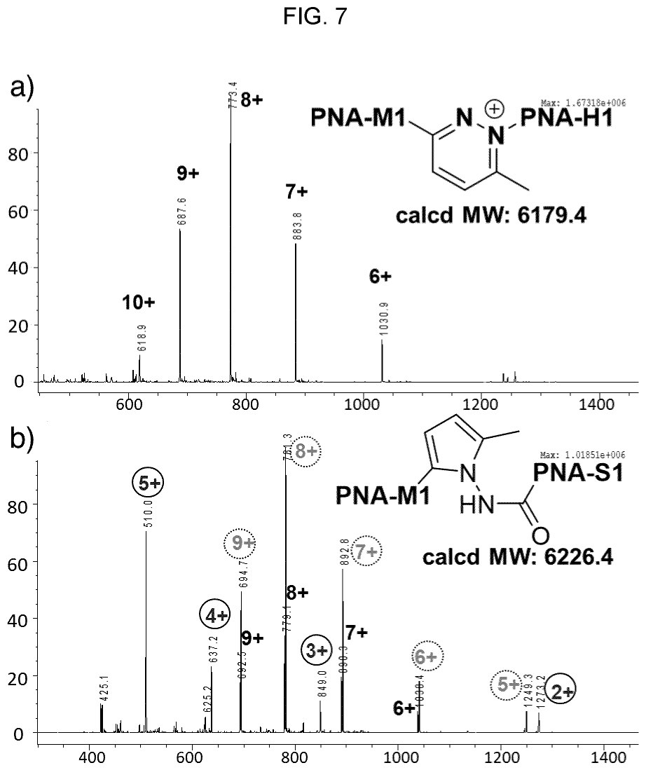

The USDS-PAGE (FIG. 6) and HPLC-UV (results not shown) analyses of the PNA-PNA ligation experiments showed the formation of a ligation product when highly reactive α-effect nucleophiles were installed on the target PNA strand and fully complementary PNA-M1 was employed (FIG. 6c for hydrazine-modified PNA-H1, FIG. 6d for hydrazide-modified PNA-Z1, and FIG. 6e for semicarbazide-modified PNA-S1). Ligation product confirmation was obtained through HPLC-ESI-MS for fully matched PNA-M1 and hydrazine-modified PNA-H1 (FIG. 7a) and for fully matched PNA-M1 and semicarbazide-modified PNA-S1 (FIG. 7b). For hydrazide-modified PNA-Z1, when combined with PNA-M1, a band corresponding to a possible ligation product was visible in the PAGE analysis but only a minor signal could be seen on the HPLC-UV, while no product formation was detected after ESI-MS analysis. Therefore, the PAGE band may be an unstable hydrazone adduct that cannot survive under chromatographic conditions. Performing multiple freeze-thawing cycles or using higher concentration (starting from 100 μM and slowly removing the solvent over a period of few days) did not change the reaction pattern.

Further, the kinetics of the reaction in the PNA-PNA ligation setup was investigated. Monitoring the reaction at 5 μM nucleophilic probe concentration in presence of a slight excess (about 1.1 eq.) of PNA-M1, it was found that reaction with the hydrazine containing PNA-H1 the ligation reaction proceeded with a faster conversion profile as compared to similar reactions in presence of the semicarbazide-containing PNA-S1. These data were obtained low μM concentrations, neutral pH, and without the addition of catalysts.

### Example 3: Methods for Covalently Binding a First Recognition Element and a Second Recognition Element According to Embodiments of the Invention in the Presence of a Third Recognition Element

In order to evaluate the possibility to form a ligation product between the DOP-modified PNA-M2 and PNA-M3 strands and the hydrazine-containing PNA-H1, two fully complementary DNA templating strands were used as a third recognition element to generate the required proximity. For PNA-M2 and PNA-H1 ligation, fully matched (FM) oligonucleotide DNA-4, 2 bases mismatched (2MM) oligonucleotide DNA-5, and 3 bases mismatched (3MM) oligonucleotide DNA-6 were generated (Table 3). For PNA-M3 and PNA-H1 ligation, fully matched (FM) oligonucleotide DNA-1, 2 bases mismatched (2MM) oligonucleotide DNA-2, and 3 bases mismatched (3MM) oligonucleotide DNA-3 were generated (Table 3). In a typical experiment, 100 μL of buffered solutions (PBS pH 7.4) containing all agents or third recognition elements at 5 μM concentration (from a 100 μM stock solution), were prepared in a 0.5 mL Eppendorf and allowed to react overnight at 25° C. The complex between DNA and the nucleophilic PNA was allowed to equilibrate for 5 minutes before the final addition of DOP-PNA. The solutions were collected in the morning and analysed via HPLC-UV, HLC-MS and USDS-PAGE.

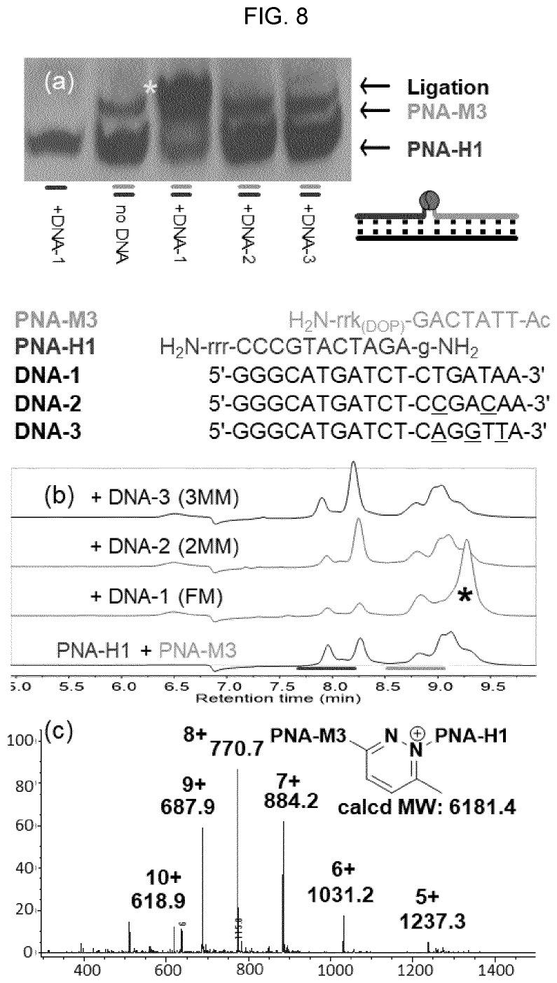

After overnight incubation of the first agent (i.e. the first PNA) and the second agent (i.e. the second PNA) with different DNA sequences as a third recognition element, formation of the desired ligation product could be observed only when the fully complementary DNA were employed, as confirmed by USDS-PAGE (FIG. 8a), HPLC (FIG. 8b) and ESI-MS (FIG. 8c) for PNA-M3 and PNA-H1 ligation. Results for PNA-M2 and PNA-H1 ligation were similar but are not shown.

TABLE 3
DNA molecules used as a third recognition
element in the methods according to an embodiment
of the invention; the underlined bases are
mutations, introducing mismatches in the binding
between the recognition elements.

| DNA | Sequence | SEQ ID NO: |
|---|---|---|
| DNA-1 | 5′-GGGCATGATCT-CTGATAA-3′ | 19 |
| DNA-2 | 5′-GGGCATGATCT-CCGACAA-3′ | 20 |
| DNA-3 | 5′-GGGCATGATCT-CAGGTTA-3′ | 21 |
| DNA-4 | 5′-GGGCATGATCT-ACATGAT-3′ | 22 |
| DNA-5 | 5′-GGGCATGATCT-ATATAAT-3′ | 23 |
| DNA-6 | 5′-GGGCATGATCT-ATAAGCT-3′ | 24 |

### Example 4: Method for Covalently Binding a First Agent and a Second Agent According to an Embodiment of the Invention Using Peptide Nucleic Acids as First and Second Recognition Elements, and an Oligonucleotide as Third Recognition Element

Materials and Methods

96-Well Plate Functionalization

Pierce Amine-binding, maleic anhydride activated, 96-well plate strips (Thermo-Fisher) where used as solid support. Functionalization was performed overnight at 300 RPM (orbital shacking) with 100 μL of a 500 nM PNA solution in 100 mM carbonate buffer pH 9.0 containing 20% acetonitrile and 0.0001% SDS. Unreacted sites were quenched using a 50 mM ethanolamine solution in 100 mM carbonate buffer pH 9.0 (300 μL, 260 RPM, 2 h). Finally, surfaces were washed with a 0.01% SDS solution (2 minutes, twice), 0.001% SDS solution (2 minutes, twice) and mQ. The 96 well plates were then dried and stored over CaCl₂.

Surface Template Ligation in 96-Well Plate

Oligonucleotides and PNA solutions were freshly prepared in PBS pH 7.4 supplemented with 0.001% SDS (PBS-S) from a 10 μM stock solutions in mQ. Surfaces were pre-wetted for 30 minutes with a 0.001% SDS solution. Then, 50 μL of oligonucleotide and 50 μL of 1 μM PNA solutions were allowed to react overnight at 40° C. Wells were washed with a mQ/MeCN 1:1+0.1% TFA solution (4×5 minutes, 45° C.) before the quantification of the attached biotin, using 100 μL of 20 ng/mL Pierce High Sensitivity NeutrAvidin-HRP-conjugate (Thermo Scientific) and 1-step Ultra TMB-ELISA (Thermo Scientific) as reagent solution. Final readout of the oxidized TMB was performed monitoring the absorption at 450 nm after quenching the reaction with 2M H₂SO₄.

Microarray Slide Functionalization

NHS-active ester XL-CX slides (Xantec, Germany) where used as solid support. Functionalization was performed spotting 0.3 μL of a 1 μM PNA solution in 100 mM carbonate buffer pH 9.0 containing 30% glycol and 0.0001% SDS. Functionalization was allowed overnight in a humid chamber (75% relative humidity) and remaining active sites were quenched for 4 h using a 6% ethanolamine solution in 100 mM carbonate buffer pH 9.0. Finally, surfaces were washed with a 0.01% SDS solution (10 minutes, twice), 0.001% SDS solution (10 minutes, twice) and mQ. Slides were dried with a stream of clean air and stored over CaCl₂. All steps were performed away from direct light.

Surface Template Ligation on Microarray Slides

Oligonucleotide and PNA solutions were freshly prepared in PBS-S from a 10 μM stock solution in mQ. Surfaces were pre-wetted for 30 minutes with a 0.001% SDS solution and then dried with a stream of clean air, before the application of the desired mask (in a typical experiment a 16 well mask is employed). 50 μL of oligonucleotide and 50 μL of 100 nM PNA solutions were added and allowed to react overnight at 40° C. Slides were washed in PBS pH 7.4, supplemented with 0.05% TWEEN-20 (2×10 minutes, 50° C.) and mQ (1 minute, r.t.). Slides were then dried with a stream of clean air before image acquisition. All steps were performed away from direct light.

Introduction and Results

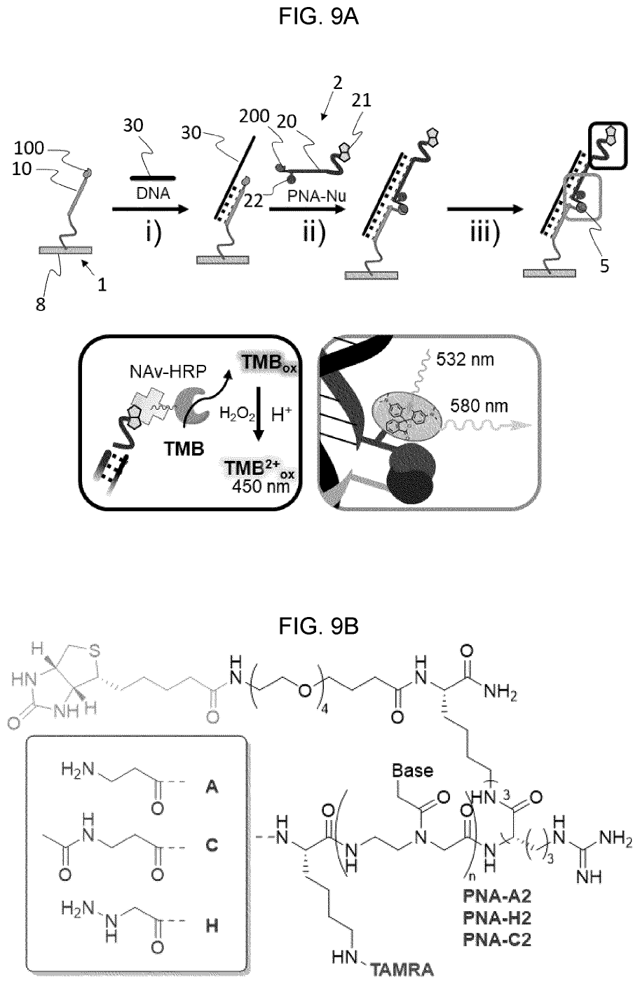

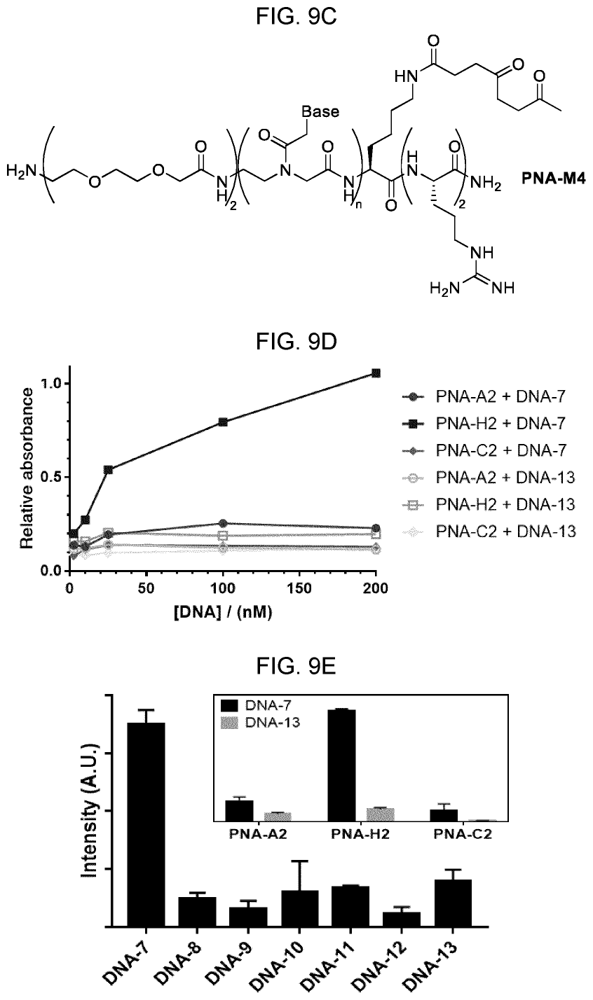

The possibility to perform the hydrazine-DOP ligation on surface was tested by exploiting the possibility to quantify the formation of the ligation product by recognition of a biotin-tag via a neutravidin-horseradish peroxidase (NAv-HRP) conjugate and measurement of the resulting peroxidase activity via TMB oxidation, or by direct quantification using a fluorescent 5-carboxytetramethylrhodamine (TAMRA)-tag, in a 96-well plate or microarray glass surface setup respectively (the complete reaction and detection scheme is depicted in FIG. 9A).

FIG. 9A provides a scheme depicting a method according to an embodiment of the invention for covalently binding a first agent (1) and a second agent (2) using a third recognition element (30), wherein the first agent comprises a surface (8) a surface (8) covalently bound to a first recognition element (1), for example a first PNA. The first recognition element (10) comprises a 1,4-dioxo moiety (100) as taught herein. The second agent (2) is a small molecule (21) such as biotin (used as an affinity tag) which is covalently bound to a second recognition element (20), for example a second PNA. The second recognition element (20) comprises a nucleophilic moiety (200) as taught herein and a fluorescent group (22), such as TAMRA. In step i), the first agent (1) is contacted with a third recognition element (30) which is an oligonucleotide comprising a sequence complementary to the oligonucleotide sequence of the first and second PNAs. In step ii), the surface is contacted with the second agent (2). Upon contacting the second agent (2) and the third recognition element (30), the second PNA binds to the third recognition element, thereby bringing in the 1,4 dioxo moiety of the first PNA in proximity to the nucleophilic moiety of the second PNA (PNA-PNA:DNA complex), allowing the formation of a covalent bond. The result is a functionalised surface, which can be visualised using the affinity tag or the fluorescent group. In step iii), the formation of the final ligation product can be performed through incubation with NAv-HRP and evaluation of the resulting peroxidase activity (black box) or by fluorescence emission of the TAMRA reporting group (grey box).

A longer DOP-containing PNA-M4 (Table 2 and FIG. 9C) was synthesized and used for surface functionalization. The surface-ligation selectivity toward the formation of a ligation product was evaluated in the presence of a target DNA sequence (Table 4) and different nucleophilic PNAs (Table 2 and FIG. 9B).

On the 96-well plate format, without any special optimization or extremely harsh washing steps, complete sequence selectivity was obtained in the low nM range, providing at the same time a confirmation of reaction chemo-selectivity on surface (as shown in FIG. 9D). Interestingly, using 200 nM of target DNA in presence of a 1 μM solution of PNA-H2, it was possible to obtain complete conversion of the probes on the surface to the desired ligation product, as the generated TMBₒₓ signal was as intense as the one generated in the positive well, where the biotin-containing PNA-A2 was directly linked to the well surface. Interestingly, similar results were also obtained on a microarray glass surface using 50 nM PNA and DNA probe concentration (FIG. 9E).

TABLE 4
DNA molecules used as a third recognition element in the methods according to
an embodiment of the invention; the underlined bases are mutations, introducing
mismatches in the binding between the recognition elements.

| DNA | Sequence | Complementarity* | SEQ ID NO: |
|---|---|---|---|
| DNA-7 | 5′-GGGCATGATCT-CTGCTCAAGAC-3′ | FM-FM | 25 |
| DNA-8 | 5′-GGGCATGATCT-CCGCGTAATAC-3′ | FM-MM | 26 |
| DNA-9 | 5′-GGCCAGGATTT-CTGCTCAAGAC-3′ | MM-FM | 27 |
| DNA-10 | 5′-GGCCAGGATTT-CCGCGTAATAC-3′ | MM-MM | 28 |
| DNA-11 | 5′-GGGCATGATCT-TACACGCTAGC-3′ | FM-scr | 29 |
| DNA-12 | 5′-AATGTGTGCGC-CTGCTCAAGAC-3′ | scr-FM | 30 |
| DNA-13 | 5′-AATGTGTGCGC-TACACGCTAGC-3′ | scr-scr | 31 |

*FM: fully matched; MM: mismatched; scr: scrambled

### Example 5: Method for Covalently Binding a First Recognition Element and a Second Recognition Element According to an Embodiment of the Invention Using Peptides as First and Second Recognition Elements

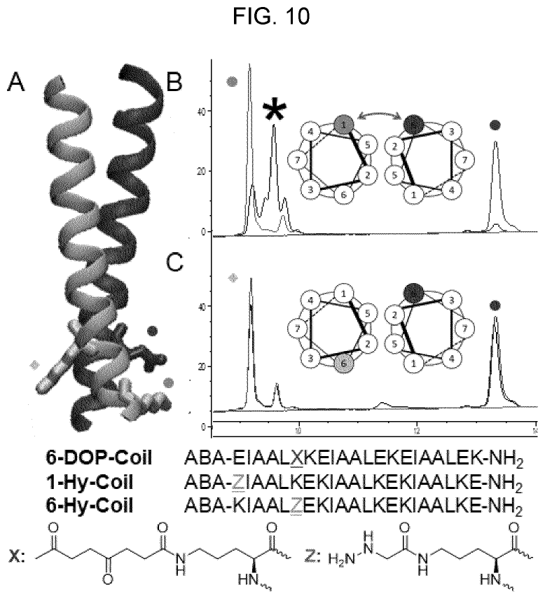

In order to broaden the scope of the proximity induced ligation, the possibility to translate this approach to peptide-peptide ligation was investigated, exploiting the supramolecular recognition of α-helix coiled coils. As a model system, a synthetic heterodimeric coil system was selected able to form a parallel coiled-coil structure, also employed for the dimerization of the Kar3Vik1 protein. The original structure of the two peptides was modified in order to accommodate the two required functional groups. In particular, in order to minimize the perturbation induced by the sequence modification, the DOP moiety was appended on an ornithine side chain replacing a glutamic acid in position 6 of the first heptad of the E-coil (6-DOP-Coil), whereas an hydrazine-modified ornithine was exploited for the replacement of either the lysine in position 1 or 6 of the first heptad of the K-coil strand (1-Hy-Coil and 6-Hy-Coil respectively) (Table 2 and FIG. 10A).

In a typical experiment, 100 μL of buffered solutions (PBS pH 7.4) containing the first recognition element at 5 μM concentration (from a 100 μM stock solution) and a second recognition element at 5 μM concentration (from a 100 μM stock solution), were prepared in a 0.5 mL Eppendorf and allowed to react overnight at 25° C. The solutions were collected in the morning and analysed via HPLC-UV and HPLC-MS.

As shown in FIG. 10B, upon overnight incubation of the two coils at 5 μM concentration, selective formation of the desired ligation product of 6-DOP-Coil only occurs in presence of 1-Hy-Coil, where the required hydrazine function is correctly oriented on the same side of the coiled-coil structure. No ligation was observed when 6-Hy-Coil was used as second recognition element (FIG. 10C).

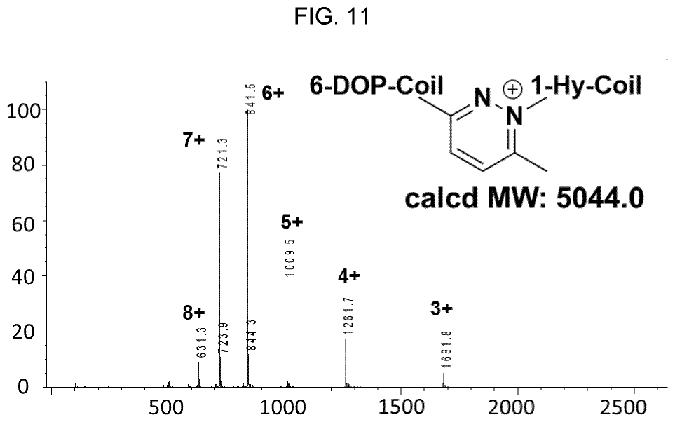

FIG. 11 shows the ESI-MS characterization of the ligation product formed between 6-DOP-Coil and 1-Hy-Coil.

Results were further validated on hydrazine-containing R-coils (Table 2), and the selectivity of the ligation was also confirmed with in presence of a short random peptide, Hy-Random (Table 2) (results not shown).

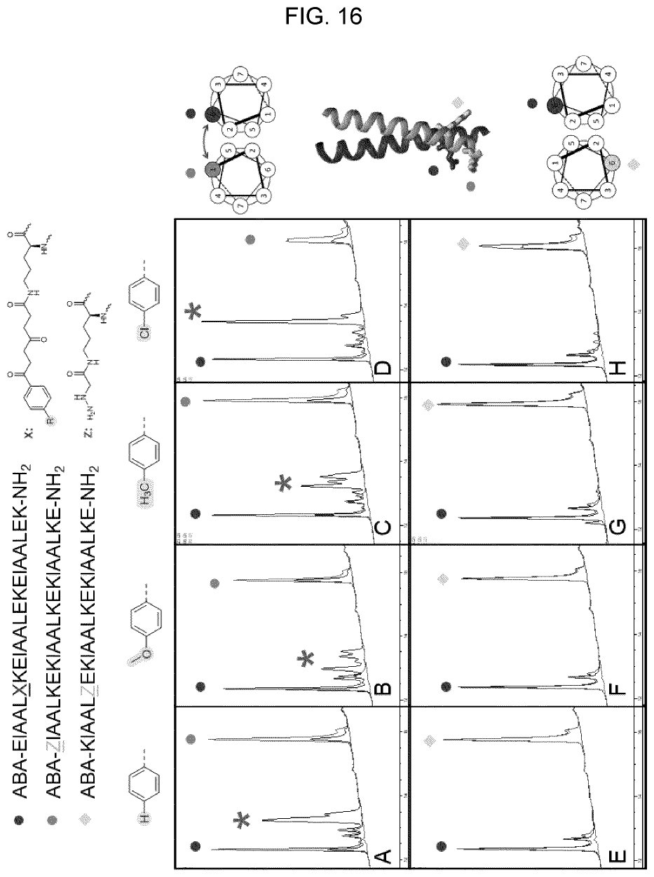

In a further experiment, a first recognition element being a peptide comprising an optionally substituted phenyl 1,4-dioxo moiety was tested (6-Xn-Coils, SEQ ID NO: 32, 33, 34, or 35 for n=1, 2, 3, or 4, respectively). The optionally substituted phenyl 1,4-dioxo moiety was appended on an ornithine side chain replacing a glutamic acid in position 6 of the first heptad of the E-coil, whereas a hydrazine-modified ornithine was exploited for the replacement of either the lysine in position 1 or 6 of the first heptad of the K-coil strand (1-Hy-Coil and 6-Hy-Coil respectively) (Table 2 and FIG. 16). The following groups were appended on an ornithine side chain: X1: (4,7-dioxo-7-phenyl-heptanoyl); X2: [7-(4-methoxyphenyl)-4,7-dioxo-heptanoyl]; X3: [4,7-dioxo-7-(p-tolyl)heptanoyl]; X4: [7-(4-chlorophenyl)-4,7-dioxo-heptanoyl] (Table 2 and FIG. 16).

In the experiment, 100 μL of buffered solutions (PBS pH 7.4) containing the first recognition element at 5 μM concentration (from a 100 μM stock solution) and the second recognition element at 5 μM concentration (from a 100 μM stock solution), were prepared in a 0.5 mL Eppendorf and allowed to react overnight at 25° C. The solutions were collected in the morning and analysed via HPLC-UV and HPLC-MS.

As shown in FIG. 16A-D, upon overnight incubation of the two coils at 5 μM concentration, selective formation of the desired ligation product of 6-Xn-Coil (n=1, 2, 3, or 4) only occurred in presence of 1-Hy-Coil, where the required hydrazine function was correctly oriented on the same side of the coiled-coil structure. No ligation was observed when 6-Hy-Coil was used as second recognition element (FIG. 16E-H).

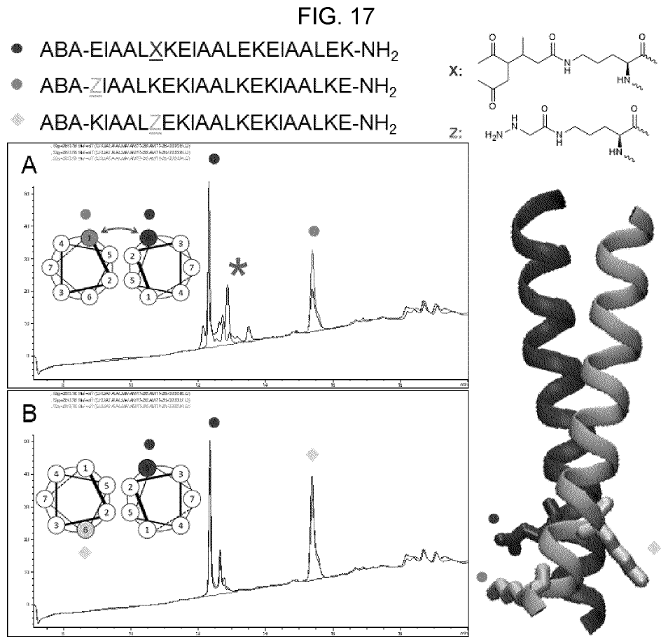

In yet another experiment, a first recognition element being a peptide comprising an 1,4-dioxo moiety having a structure of Formula IC (6-X5-Coil) was used. The (4-acetyl-3-methyl-6-oxo-heptanoyl) moiety was appended on an ornithine side chain replacing a glutamic acid in position 6 of the first heptad of the E-coil (6-X5-Coil, Table 2 and FIG. 17), whereas an hydrazine-modified ornithine was exploited for the replacement of either the lysine in position 1 or 6 of the first heptad of the K-coil strand (1-Hy-Coil and 6-Hy-Coil respectively) (Table 2 and FIG. 17).

In the experiment, 100 μL of buffered solutions (PBS pH 7.4) containing the first recognition element at 5 μM concentration (from a 100 μM stock solution) and a second recognition element at 5 μM concentration (from a 100 μM stock solution), were prepared in a 0.5 mL Eppendorf and allowed to react overnight at 25° C. The solutions were collected in the morning and analysed via HPLC-UV and HPLC-MS.

As shown in FIG. 17A, upon overnight incubation of the two coils at 5 μM concentration, selective formation of the desired ligation product of 6-X5-Coil only occurred in presence of 1-Hy-Coil, where the required hydrazine function was correctly oriented on the same side of the coiled-coil structure. No ligation was observed when 6-Hy-Coil was used as second recognition element (FIG. 17B).

### Example 6: Method for Covalently Binding a First Agent and a Second Agent According to an Embodiment of the Invention Using Peptide Nucleic Acids as First and Second Recognition Elements

Solution Ligation of Proteins

In a typical experiment, 100 μL of buffered solutions (PBS pH 7.4) containing each PNA-protein construct at 1 μM concentration (from a 10 μM stock solution in PBS or any other compatible buffer), are prepared in a 0.2 mL Eppendorf and allowed to react overnight at 4° C. The solutions are collected in the morning and analyzed via HPLC-UV, HPLC-MS, and USDS-PAGE. Purification of the protein-protein adduct can be obtained through gel-filtration purification.

Surface Ligation of a Protein on Microarray Slides

A PNA-protein solution is freshly prepared in 0.05% TWEEN-20 in PBS from a 10 μM stock solution in PBS. Surfaces are pre-wetted for 30 minutes with a 0.05% TWEEN-20 solution and then dried with a stream of clean air, before the application of the desired mask (in a typical experiment a 32 well mask is employed). 50 μL of 100 nM probe solution is added and allowed to react overnight at 4° C. Slides are washed in PBS pH 7.4, supplemented with 0.05% TWEEN-20 (2×10 minutes, 25° C.) and PBS (1 minute, 25° C.). The protein is bound on the surface as illustrated in FIG. 15.

### CONCLUSION

In summary, a proximity-induced ligation methodology has been developed and applied for the ligation of first and second agents using PNA and peptide recognition elements under catalyst-free physiologically relevant conditions, exploiting different architectures and demonstrating its feasibility for both solution and surface approaches. This ligation, given the simplicity, robustness, and complete bio-orthogonality of the functional groups involved, can foster the development of new enzyme-free applications were a selective ligation of different partners is required.

1. A method for covalently binding a first agent and a second agent, the first agent comprising a first recognition element, wherein the first recognition element comprises an 1,4-dioxo moiety having a structure of Formula IA, IB or IC, wherein R¹² is C₁₋₃₀alkyl, C₂₋₃₀ alkenyl, C₆₋₁₅aryl, or C₅₋₁₅heteroaryl, wherein the C₁₋₃₀ alkyl, C₂₋₃₀ alkenyl, C₆₋₁₅ aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀ alkyl or C₂₋₃₀ alkenyl; 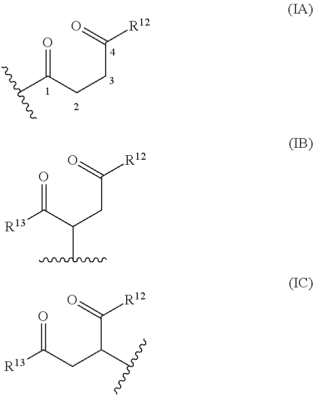
the second agent comprising a second recognition element, wherein the second recognition element comprises a nucleophilic moiety selected from a hydrazine moiety, an aminooxy moiety, an aminosulfanyl moiety or a hydroxylamine moiety; wherein
(A) the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the first recognition element and the second recognition element are capable of non-covalently binding to each other such that the 1,4-dioxo moiety and the nucleophilic moiety are brought in proximity; the method comprising contacting the first agent with the second agent, thereby covalently binding the 1,4-dioxo moiety and the nucleophilic moiety; or
(B) the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, the third recognition element is a nucleic acid, an oligonucleotide, an oligonucleotide mimic, a PNA, a protein, a peptide, a cyclodextrin, a cucurbituril, a cyclophane, or a combination thereof, and the first recognition element and the second recognition element are capable of non-covalently binding to a third recognition element such that the 1,4-dioxo moiety and the nucleophilic moiety are brought in proximity; the method comprising contacting the first agent with the second agent and the third recognition element, thereby covalently binding the 1,4-dioxo moiety and the nucleophilic moiety.

2. The method according to claim 1, wherein the first recognition element and the second recognition element are capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å.

3. The method according to claim 1, wherein the first recognition element and the second recognition element are capable of non-covalently binding to a third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å.

4. The method according to claim 1, comprising the prior steps of:
providing an agent comprising a first recognition element, wherein the first recognition element comprises a furyl moiety having a structure of Formula IIA, IIB, or IIC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl; 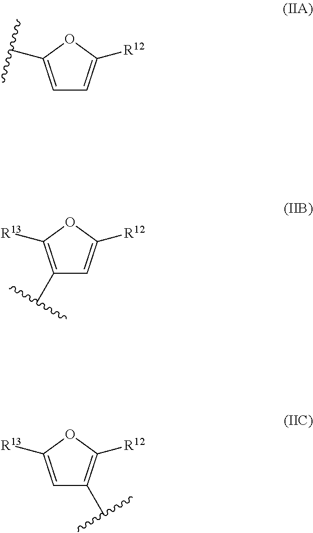
hydrolysing the furyl moiety, thereby obtaining the first agent.

5. The method according to claim 1, wherein R¹² is C₁₋₃₀alkyl or C₃₋₃₀ alkenyl, and R¹³ if present is hydrogen, C₁₋₃₀alkyl or C₃₋₃₀ alkenyl; or wherein R¹² is methyl, and R¹³, if present, is hydrogen or methyl.

6. The method according to claim 1, wherein the first recognition element and the second recognition element are complementary peptide nucleic acids; wherein the first recognition element and the second recognition element are coiled coil peptides; or wherein the first recognition element and the second recognition element are peptide nucleic acids, and the third recognition element is an oligonucleotide, wherein the first recognition element and the second recognition element are complementary to the third recognition element.

7. The method according to claim 1, wherein the first recognition element comprises an 1,4-dioxo moiety having a structure of Formula IA, wherein R¹² is C₁₋₃₀ alkyl, C₂₋₃₀alkenyl, C₆₋₁₅aryl, or C₅₋₁₅heteroaryl, wherein the C₁₋₃₀ alkyl, C₂₋₃₀ alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy.

8. The method according to claim 1, wherein the first recognition element comprises a 1,4-dioxo moiety having a structure of Formula III or IIIa, wherein: 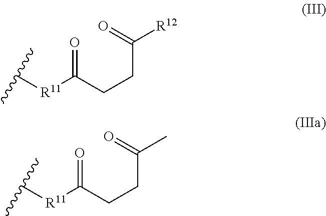
R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; and
R¹² is C₁₋₃₀alkyl, C₂₋₃₀alkenyl, C₆₋₁₅aryl, or C₅₋₁₅heteroaryl, wherein the C₁₋₃₀ alkyl, C₂₋₃₀ alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy.

9. The method according to claim 1, wherein the nucleophilic moiety comprises a group having a structure of Formula VII, VIII, or IX, wherein: 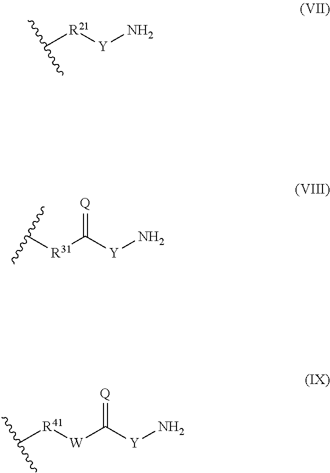
Y is NR¹, O or S, wherein R¹ is hydrogen, C₁₋₃₀alkyl, or C₆₋₂₀aryl;
R²¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
R³¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
Q is O, S, or NR⁴, wherein R⁴ is hydrogen, C₁₋₃₀alkyl or C₆₋₂₀aryl;
R⁴¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy;
W is NR⁵, O, or S, wherein R⁵ is hydrogen, C₁₋₃₀alkyl or C₆₋₂₀aryl.

10. The method according to claim 1, wherein the first agent and/or the second agent is a protein, a polypeptide, a peptide, a peptide nucleic acid (PNA), a peptidomimetic, a nucleic acid, an oligonucleotide, an oligonucleotide mimic, a polysaccharide, a lipid, a small molecule, a polymer, a labeling reagent, a solid surface, a particle, or a combination thereof.

11. The method according to claim 1, wherein the first agent is contacted with the second agent, and optionally the third recognition element, without the addition of an activation signal.

12. A kit of parts comprising:
a) a first recognition element comprising an 1,4-dioxo moiety having a structure of Formula IA, IB, or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅ aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl; and
b) a second recognition element comprising a hydrazine moiety, an aminooxy moiety, an aminosulfanyl moiety or a hydroxylamine moiety;
wherein the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the first recognition element and the second recognition element are capable of non-covalently binding to each other such that the 1,4-dioxo moiety and the hydrazine moiety, aminooxy moiety, aminosulfanyl moiety or hydroxylamine moiety are brought in proximity.

13. A kit of parts comprising:
a′) a first recognition element comprising an 1,4-dioxo moiety having a structure of Formula IA, IB, or IC, wherein R¹² is C₁₋₃₀alkyl, C₂₋₃₀alkenyl, C₆₋₁₅ aryl, or C₅₋₁₅heteroaryl, wherein the C₁₋₃₀alkyl, C₂₋₃₀alkenyl, C₆₋₁₅ aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl;
b′) a second recognition element comprising a hydrazine moiety, an aminooxy moiety, an aminosulfanyl moiety or a hydroxylamine moiety;
c′) a third recognition element capable of non-covalently binding to the first recognition element and the second recognition element such that the 1,4-dioxo moiety and the hydrazine moiety, aminooxy moiety, aminosulfanyl moiety or hydroxylamine moiety are brought in proximity;
wherein the first recognition element is a peptide nucleic acid (PNA), a peptide, a peptidomimetic, an oligonucleotide, an oligonucleotide mimic, or a combination thereof, the second recognition element is a PNA, a peptide, a peptidomimetic, an oligonucleotide, an oligonucleotide mimic, or a combination thereof, and the third recognition element is a nucleic acid, an oligonucleotide, an oligonucleotide mimic, a PNA, a protein, a peptide, a cyclodextrin, a cucurbituril, a cyclophane, or a combination thereof.

14. The kit of parts according to claim 12, wherein the first recognition element and the second recognition element are capable of non-covalently binding to each other such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å, or wherein the first recognition element and the second recognition element are capable of non-covalently binding to a third recognition element such that the distance between: (i) the carbon atom at position 1 or 4 of the 1,4-dioxo moiety and (ii) the terminal nitrogen atom of the hydrazine moiety, the aminooxy moiety or the aminosulfanyl moiety or (ii′) the terminal oxygen atom of the hydroxylamine moiety is at most 10 Å.

15. The kit of parts according to claim 12, wherein:
the first recognition element and the second recognition element are complementary peptide nucleic acids, or wherein the first recognition element and the second recognition element are coiled coil peptides;
the first recognition element and the second recognition element are peptide nucleic acids, and the third recognition element is an oligonucleotide, wherein the first recognition element and the second recognition element are complementary to the third recognition element;
R¹² is C₁₋₃₀alkyl or C₃₋₃₀alkenyl, and R¹³ if present is hydrogen, C₁₋₃₀alkyl or C₃₋₃₀alkenyl;
R¹² is methyl, and R¹³, if present, is hydrogen or methyl;
the 1,4-dioxo moiety comprises a group having a structure of Formula III or IIIa, wherein:
R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; and
R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and/or
the nucleophilic moiety comprises a group having a structure of Formula IV, V, or IV, wherein:
Y is NR¹, O or S, wherein R¹ is hydrogen, C₁₋₃₀alkyl, or C₆₋₂₀aryl;
R²¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy;
R³¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy;
Q is O, S, or NR⁴, wherein R⁴ is hydrogen, C₁₋₃₀alkyl or C₆₋₂₀aryl;
R⁴¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆ cycloalkyl, carboxyl, or C₁₋₆alkoxy;
W is NR⁵, O, or S, wherein R⁵ is hydrogen, C₁₋₃₀alkyl or C₆₋₂₀aryl.

16. A peptide nucleic acid (PNA) comprising an 1,4-dioxo moiety having a structure of Formula IA, IB, or IC, wherein R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.

17. A peptide comprising an 1,4-dioxo moiety having a structure of Formula IA, IB, or IC, wherein R¹² is C₁₋₃₀alkyl, C₂₋₃₀alkenyl, C₆₋₁₅ aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy; and R¹³, if present, is hydrogen, C₁₋₃₀alkyl or C₂₋₃₀alkenyl.

18. The PNA according to claim 16, wherein:
R¹² is C₁₋₃₀alkyl or C₃₋₃₀alkenyl, and R¹³ if present is hydrogen, C₁₋₃₀alkyl or C₃₋₃₀alkenyl;
R¹² is methyl, and R¹³, if present, is hydrogen or methyl; and/or
the PNA or the peptide comprises a group having a structure of Formula III or IIIa, wherein:
R¹¹ is C₁₋₁₅alkyl, C₃₋₁₅alkenyl, C₆₋₁₅aryl, or C₅₋₁₅heteroaryl, wherein the C₁₋₁₅alkyl, C₆₋₁₅aryl, or C₅₋₁₅heteroaryl group are optionally substituted with an C₁₋₆alkyl, C₃₋₆cycloalkyl, carboxyl, or C₁₋₆alkoxy; and
R¹² is C₁₋₃₀alkyl, C₂₋₃₀alkenyl, C₆₋₁₅aryl, or C₅₋₁₅heteroaryl, wherein the C₁₋₃₀alkyl, C₂₋₃₀alkenyl, C₆₋₁₅aryl, or C₅₋₁₅heteroaryl group are optionally substituted with an C₁₋₆alkyl, C₃₋₆cycloalkyl, halogen, amine, hydroxyl, sulfhydryl, carboxyl, or C₁₋₆alkoxy.


SEQUENCE LISTING (36 sequences)

SEQ ID NO: 1 - DNA, 10 bp, Artificial Sequence
  PNA-M1
  modified_base (1)..(7): N-(2-aminoethyl) glycine nucleoside
  misc_feature (1)..(1): acetylation
  misc_feature (8)..(8): Lys modified on the side chain with 4,7- dioxooctanoyl
  misc_feature (9)..(10): Arg
atgatctnnn

SEQ ID NO: 2 - DNA, 10 bp, Artificial Sequence
  PNA-M2
  misc_feature (1)..(1): modification by acetylation
  modified_base (1)..(7): N-(2-aminoethyl) glycine nucleoside
  misc_feature (8)..(8): Lys modified on the side chain with 4,7- dioxooctanoyl
  misc_feature (9)..(10): Arg
atcatgtnnn

SEQ ID NO: 3 - DNA, 10 bp, Artificial Sequence
  PNA-M3
  misc_feature (1)..(1): modification by acetylation
  modified_base (1)..(7): N-(2-aminoethyl) glycine nucleoside
  misc_feature (8)..(8): Lys modified on the side chain with 4,7- dioxooctanoyl
  misc_feature (9)..(10): Arg
ttatcagnnn

SEQ ID NO: 4 - DNA, 14 bp, Artificial Sequence
  PNA-M4
  modified_base (1)..(11): N-(2-aminoethyl) glycine nucleoside
  misc_feature (1)..(1): modification by -OOH with O being 2-(2- aminoethoxy)ethoxyacetyl
  misc_feature (12)..(12): Lys modified on the side chain with 4,7- dioxooctanoyl
  misc_feature (13)..(14): Arg
gtcttgagcagnnn

SEQ ID NO: 5 - DNA, 16 bp, Artificial Sequence
  PNA-A1
  misc_feature (1)..(1): H-beta-Ala
  misc_feature (2)..(2): Lys modified on the side chain with acetyl
  modified_base (3)..(13): N-(2-aminoethyl) glycine nucleoside
  misc_feature (14)..(16): Arg
nnagatcatgcccnnn

SEQ ID NO: 6 - DNA, 16 bp, Artificial Sequence
  PNA-C1
  misc_feature (1)..(1): acetyl-beta-Ala
  misc_feature (2)..(2): Lys modified on the side chain with acetyl
  modified_base (3)..(13): N-(2-aminoethyl) glycine nucleoside
  misc_feature (14)..(16): Arg
nnagatcatgcccnnn

SEQ ID NO: 7 - DNA, 16 bp, Artificial Sequence
  PNA-H1
  misc_feature (1)..(1): H2N-Gly
  misc_feature (2)..(2): Lys modified on the side chain with acetyl
  modified_base (3)..(13): N-(2-aminoethyl) glycine nucleoside
  misc_feature (14)..(16): Arg
nnagatcatgcccnnn

SEQ ID NO: 8 - DNA, 15 bp, Artificial Sequence
  PNA-Z1
  misc_feature (1)..(1): H2N-NHCO(CH2)2CO-Lys modified on the side chain with acetyl
  modified_base (2)..(12): N-(2-aminoethyl) glycine nucleoside
  misc_feature (13)..(15): Arg
nagatcatgcccnnn

SEQ ID NO: 9 - DNA, 15 bp, Artificial Sequence
  PNA-S1
  misc_feature (1)..(1): H2N-NHCONH-CH2CO-Lys modified on the side chain with acetyl
  modified_base (2)..(12): N-(2-aminoethyl) glycine nucleoside
  misc_feature (13)..(15): Arg
nagatcatgcccnnn

SEQ ID NO: 10 - DNA, 16 bp, Artificial Sequence
  PNA-A2
  misc_feature (1)..(1): H-beta-Ala
  misc_feature (2)..(2): Lys modified on the side chain with 5- carboxytetramethylrhodamine
  modified_base (3)..(13): N-(2-aminoethyl) glycine nucleoside
  misc_feature (14)..(16): Arg
nnagatcatgcccnnn

SEQ ID NO: 11 - DNA, 16 bp, Artificial Sequence
  PNA-H2
  misc_feature (1)..(1): H2N-Gly
  misc_feature (2)..(2): Lys modified on the side chain with 5- carboxytetramethylrhodamine
  modified_base (3)..(13): N-(2-aminoethyl) glycine nucleoside
  misc_feature (14)..(16): Arg
nnagatcatgcccnnn

SEQ ID NO: 12 - DNA, 16 bp, Artificial Sequence
  PNA-C2
  misc_feature (1)..(1): acetyl-beta-Ala
  misc_feature (2)..(2): Lys modified on the side chain with 5- carboxytetramethylrhodamine
  modified_base (3)..(13): N-(2-aminoethyl) glycine nucleoside
  misc_feature (14)..(16): Arg
nnagatcatgcccnnn

SEQ ID NO: 13 - protein, 21 aa, Artificial Sequence
  6-DOP-coil
  PEPTIDE (16)..(16): ornithine modified on the side chain with 4,7- dioxooctanoyl
  PEPTIDE (21)..(21): modification of the C-terminus by 4- acetamidobenzoyl
KELAAIEKELAAIEKXLAAIE

SEQ ID NO: 14 - protein, 21 aa, Artificial Sequence
  1-Hy-coil
  PEPTIDE (21)..(21): ornithine modified on the side chain with H2N- Gly
  PEPTIDE (21)..(21): modification of the C-terminus by 4- acetamidobenzoyl
EKLAAIKEKLAAIKEKLAAIX

SEQ ID NO: 15 - protein, 21 aa, Artificial Sequence
  6-Hy-coil
  PEPTIDE (16)..(16): ornithine modified on the side chain with H2N- Gly
  PEPTIDE (21)..(21): modification of the C-terminus by 4- acetamidobenzoyl
EKLAAIKEKLAAIKEXLAAIK

SEQ ID NO: 16 - protein, 21 aa, Artificial Sequence
  1-Hy-coil(R)
  PEPTIDE (21)..(21): ornithine modified on the side chain with H2N- Gly
  PEPTIDE (21)..(21): modification of the C-terminus by 4- acetamidobenzoyl
ERLAAIRERLAAIRERLAAIX

SEQ ID NO: 17 - protein, 21 aa, Artificial Sequence
  6-Hy-coil(R)
  PEPTIDE (16)..(16): ornithine modified on the side chain with H2N- Gly
  PEPTIDE (21)..(21): modification of the C-terminus by 4- acetamidobenzoyl
ERLAAIRERLAAIREXLAAIR

SEQ ID NO: 18 - protein, 8 aa, Artificial Sequence
  Hy-random
  PEPTIDE (8)..(8): ornithine modified on the side chain with H2N- Gly
YKADYGFX

SEQ ID NO: 19 - DNA, 18 bp, Artificial Sequence
  DNA-1
gggcatgatctctgataa

SEQ ID NO: 20 - DNA, 18 bp, Artificial Sequence
  DNA-2
gggcatgatctccgacaa

SEQ ID NO: 21 - DNA, 18 bp, Artificial Sequence
  DNA-3
gggcatgatctcaggtta

SEQ ID NO: 22 - DNA, 18 bp, Artificial Sequence
  DNA-4
gggcatgatctacatgat

SEQ ID NO: 23 - DNA, 18 bp, Artificial Sequence
  DNA-5
gggcatgatctatataat

SEQ ID NO: 24 - DNA, 18 bp, Artificial Sequence
  DNA-6
gggcatgatctataagct

SEQ ID NO: 25 - DNA, 22 bp, Artificial Sequence
  DNA-7
gggcatgatctctgctcaagac

SEQ ID NO: 26 - DNA, 22 bp, Artificial Sequence
  DNA-8
gggcatgatctccgcgtaatac

SEQ ID NO: 27 - DNA, 22 bp, Artificial Sequence
  DNA-9
ggccaggatttctgctcaagac

SEQ ID NO: 28 - DNA, 22 bp, Artificial Sequence
  DNA-10
ggccaggatttccgcgtaatac

SEQ ID NO: 29 - DNA, 22 bp, Artificial Sequence
  DNA-11
gggcatgatcttacacgctagc

SEQ ID NO: 30 - DNA, 22 bp, Artificial Sequence
  DNA-12
aatgtgtgcgcctgctcaagac

SEQ ID NO: 31 - DNA, 22 bp, Artificial Sequence
  DNA-13
aatgtgtgcgctacacgctagc

SEQ ID NO: 32 - protein, 21 aa, Artificial Sequence
  6-X1-coil
  PEPTIDE (16)..(16): x = ornithine modified on the side chain with (4,7-dioxo-7-phenyl-heptanoyl)
  PEPTIDE (21)..(21): modification of the C-terminus by 4- acetamidobenzoyl
KELAAIEKELAAIEKXLAAIE

SEQ ID NO: 33 - protein, 21 aa, Artificial Sequence
  6-X2-coil
  PEPTIDE (16)..(16): x = ornithine modified on the side chain with [7-(4-methoxyphenyl)-4,7-dioxo-heptanoyl]
  PEPTIDE (21)..(21): modification of the C-terminus by 4- acetamidobenzoyl
KELAAIEKELAAIEKXLAAIE

SEQ ID NO: 34 - protein, 21 aa, Artificial Sequence
  6-X3-coil
  PEPTIDE (16)..(16): x = ornithine modified on the side chain with [4,7-dioxo-7-(p-tolyl)heptanoyl]
  PEPTIDE (21)..(21): modification of the C-terminus by 4- acetamidobenzoyl
KELAAIEKELAAIEKXLAAIE

SEQ ID NO: 35 - protein, 21 aa, Artificial Sequence
  6-X4-coil
  PEPTIDE (16)..(16): x = ornithine modified on the side chain with [7-(4-chlorophenyl)-4,7-dioxo-heptanoyl]
  PEPTIDE (21)..(21): modification of the C-terminus by 4- acetamidobenzoyl
KELAAIEKELAAIEKXLAAIE

SEQ ID NO: 36 - protein, 21 aa, Artificial Sequence
  6-X5-coil
  PEPTIDE (16)..(16): x = ornithine modified on the side chain with (4-acetyl-3-methyl-6-oxo-heptanoyl)
  PEPTIDE (21)..(21): modification of the C-terminus by 4- acetamidobenzoyl
KELAAIEKELAAIEKXLAAIE